SEQUENCE LISTING (134 sequences)

SEQ ID NO: 1 - DNA, 4991 bp, Glycine max
  source 1..4991: mol_type = genomic DNA
aaatggaagagtagcgatagacagaggagagagaaagagagggagtaagtgggtaaaggaaagaaaactaaagaaacaaacctaacacaacacaaatccttgaaacgacgacggaaatggccgtttcgagctcgcacatgcgtttcacctttgagtgccgctccgatcccgatttctcgccccccccgccgtccttcgacaacctccgccgccgaaacttccgctcctccgcaggatccggcgcggcgtttcacggcatctcctccctcatcctccgcttccctcccaacttccagcgccagctaagcaccaaggcgcgccgcaactgcagcaacatcggcgtcgcgcaaatcgtcgccgcttcgtggtcgaacaacagcgacaactctccggccgccggggctccggcgccgcccgcggccaccgccacggacgccgctacggtgcctctccccgtcgtcgtcgccgccaacgaggacgtcgttgtctccgccgcggcagacgagaacggggctgtacagttaaacagtagttcttattcttcatttttgaaatccgatgcaagcaaaacgattcatgccggtacgtgaaattcttgtctttttttgcgtcattaattgttattcaagttttactttccaaaattcgattactttttaattaaattagatgtgatttccttggataatgattctttaaattgtaccttttcaatctctttatgttttgtttgatttttcattaatcattacatttggatgtgatttgcttaagaaagagattttctcttgctttttgtttgtactgtacagaattaaatactttttcttgtcttactcggtatatttattagtttatatgaagtttgatttaggatttagattagggtttttggttaatttgtatggattttaatgcagttagttttaatttggattgattttgaattcgggcaatgcagctgaaagactgggtaggggtattgagactgatggaattaccacccctgtggttaacacttctgcctacttttttaagaaaaccgctgatctcattgatttcaaggtactgttctttggactgttattaaagcttttattcgcctaatgcgacaaaggtaaatgccagttattggtgtggctaaatttctcatcatttggcaggagaatcgtcaagtgagttatgaatacgggcgctatggaaacccaacgacggtggttctggaggagaagataaggttcgtgtgttttgattttgttattttaattatcttgtaatgtgttttaggtgttggggtcatttgatgaggtttgttgtcattatttaccagtgcattggagggggccgaatcaactgtgataatggcgtctgggatgtgtgctagcgtagtcctgtttatggcactggttccagctggtggacatcttgtgaccactacggattgttataggaagactagaatattcattgagacttttcttccaaagatggggatcacggtaaatgttcttctaatatattcatttggtggtttaattgtattttgtatttgttgcatggaaactgcttcatatgttgtcacgtttctctagcttaagagttgtttaacctgtttggattcaaagaatgcttattttcttatgattcaatgcaatttggctacttaccaaatcatatcataagatgaatcttaaatgagtttgtatcttgcggtacataaatgaatccgtgtaattttatatcatcgatatgagtcacacattcattgtgtgattcatttttatttttattattatttaaacttttctttcaccaattattgtgaaaaaattgaatagtcatactagtttaaatagttaaatgaaaaggaattgaaggaacacttctgaatttatcctatttgattattcaactactttttatctttcacacaattgttcaatgcatgcactctatttacctatttaattgctttgtttctattcttcttaaataaacttcacaaactcatatataactaatattttttggtatgatatatttgaagtagtttttcttgttgaaagaatgcttcttctttttctgttgttcaaaattctttgtgcatgcataattttactcaaattattaaaagaaatctcatggtgaataatattactgagaatatggtttttggtcaagtctaatagcaggcgatgtgtgatccatagagccaacctcacctactggaatgaagttattgatgttgttgtagtctttcctagaatataatgattactattagaggcacctaatataaatgtctcactgaaaatttgcttggttttcaactgaaaaattgaaatggatatcttaaatgcgaatctacttgtcagttgatagaataaggacataaggtggacttttgacattttcaatgtgtttatttctggtaggtttttactgttgtcttgggcccttgcaattataaatttataattgttatagatttgtcatatatatgtaacatactttccctttgatgaacagaccactgtaattgatccagcagatgttggagccttggaatctgcattggagcagcacaatgttagtgttggagagagtagttggttttcttttatggaattctctgattgatttatttgtagttaatagatggtctagagttaatatggtattatacaatcgtgcaggtgtctctattcttcactgagtctcctaccaatccattcctgagatgtgttgatattaagctggtttcagagctttgccacaagaaggggactttgctctgtattgatggtacatttgcaactccattgaaccagaaggcccttgcccttggcgctgatctgattctgcactccttaacaaaatacatgggtggacatcatgatgtaagtcagcctgttctttaaaatttagtttgatggaaacaacacaaattttactatttcagtagttcatagttaagcatcaaggaatgtttttcctctttcaggtccttggtggttgcataagtggttcaattaaggtggtttcgcaaattcggactttgcaccatgttttgggtggtacacttaacccggtaagtgatttctatagggccgagcctccatggaaggatatcttaatttgttattgtggctaacatggtgccaatttctttgcagaatgctgcatacctattcatcagaggcatgaaaacgctgcatctccgtgtacagcagcagaattcaacaggaatgaggatggccaaacttttagaggcacatcccaaggtatcatggcattgatatgcacactgtctttcctcctctccatgctatttaatataatttattatttggttaattgaaatatttaaagctgttgaatgagcatgtgtgtataagtatgtttcaattgcattcaattatataaaatgaatggttaacatatgacttgtgtataatgatggttgaattgtgttctatgtaaaatgaagtatatgtttaatatgccatttcctcttgttcatcaacaacatatttatgttgaaaaatcgtgtattaccccccatatgtacactaaatgtatgtggttataggatgatggacaatgtgtactaagctgtgccaagagtgcaggtgctggtttcttttgaattttttatgttgtgagttgtgatgcacttaaatgtgcatcccttgatctgctattatcttgtatgttaatgatagtaagtaattaagtattattacttttttgatagtagcttttaggatggatgctaagtatgtctgtggcataatttctcttgaaacaaaactgtattcttctctaggttcttcctttttttctttcttttctttttcctctttttgaattatcaaatcagttgtttgtatcttttctttttctgtttggacaccattattatctaatgcatatttttataccattttatcattttacagattcaatatgtaaatattttcatgattgagtcctgtatctgatgtgacttggctttaattacatactactattggcaggtgaagcgggtctactatccaggcttgccgagtcaccctgaacatgagcttgccaagaggcagatgactggtttcggtggtgttgtcagttttgaggtgggaatttgacaagttgctattcaaacatgtggtttcctaataacactattttttttttcttttatcaagaatggtgtattggataacatcatgtgccatattttttttatagttaaattgccaatattgcagattgatggagatctacataccacaataaaatttattgattcattgaaaatcccatatattgcggcctcgtttggtggctgtgagagcattgtggatcaacctgctattttgtcttactggtactcatttggattccatgttcaatttgtcatgttgatgattttgacactacacagggaatggtcctctacttgaatgtgaccctgtggtgaaagcatctttagctggtccttttggtttgttatagtgtcaaagtttaccttgtttttgttaatttaatttaattttcaacatattgtgcaacagggatcttcctcagtcagaaagggccaagtacaagatttatgacaacctggttcgcttcagctttggagttgaagattttgaggatttgaaggctgatgtcctgcaagctctggaagctatatagacagttttcctgattcacccaagtttttttcttttataattgtgctatttgtttgttatcacatctggcgattcaattgaattttgatcgtctaatgttctgttggaattgtgttaagatgaatggtctctaatttggatgttatgaaacttgtgatgaattgttgaaattgaaacctctatttgatgatattcttacccgtttttcctacaaatatcaaagcaggatatctgagaattggaaaatgataaatggtatgaaagcagtttcgtgtgaaatttgcacgaatg

SEQ ID NO: 2 - DNA, 1611 bp, synthetic construct
  source 1..1611: mol_type = other DNA
atggccgtttcgagctcgcacatgcgtttcacctttgagtgccgctccgatcccgatttctcgccccccccgccgtccttcgacaacctccgccgccgaaacttccgctcctccgcaggatccggcgcggcgtttcacggcatctcctccctcatcctccgcttccctcccaacttccagcgccagctaagcaccaaggcgcgccgcaactgcagcaacatcggcgtcgcgcaaatcgtcgccgcttcgtggtcgaacaacagcgacaactctccggccgccggggctccggcgccgcccgcggccaccgccacggacgccgctacggtgcctctccccgtcgtcgtcgccgccaacgaggacgtcgttgtctccgccgcggcagacgagaacggggctgtacagttaaacagtagttcttattcttcatttttgaaatccgatgcaagcaaaacgattcatgccgctgaaagactgggtaggggtattgagactgatggaattaccacccctgtggttaacacttctgcctacttttttaagaaaaccgctgatctcattgatttcaaggagaatcgtcaagtgagttatgaatacgggcgctatggaaacccaacgacggtggttctggaggagaagataagtgcattggagggggccgaatcaactgtgataatggcgtctgggatgtgtgctagcgtagtcctgtttatggcactggttccagctggtggacatcttgtgaccactacggattgttataggaagactagaatattcattgagacttttcttccaaagatggggatcacgaccactgtaattgatccagcagatgttggagccttggaatctgcattggagcagcacaatgtgtctctattcttcactgagtctcctaccaatccattcctgagatgtgttgatattaagctggtttcagagctttgccacaagaaggggactttgctctgtattgatggtacatttgcaactccattgaaccagaaggcccttgcccttggcgctgatctgattctgcactccttaacaaaatacatgggtggacatcatgatgtccttggtggttgcataagtggttcaattaaggtggtttcgcaaattcggactttgcaccatgttttgggtggtacacttaacccgaatgctgcatacctattcatcagaggcatgaaaacgctgcatctccgtgtacagcagcagaattcaacaggaatgaggatggccaaacttttagaggcacatcccaaggtgaagcgggtctactatccaggcttgccgagtcaccctgaacatgagcttgccaagaggcagatgactggtttcggtggtgttgtcagttttgagattgatggagatctacataccacaataaaatttattgattcattgaaaatcccatatattgcggcctcgtttggtggctgtgagagcattgtggatcaacctgctattttgtcttactgggatcttcctcagtcagaaagggccaagtacaagatttatgacaacctggttcgcttcagctttggagttgaagattttgaggatttgaaggctgatgtcctgcaagctctggaagctatatag

SEQ ID NO: 3 - protein, 536 aa, Glycine max
MAVSSSHMRFTFECRSDPDFSPPPPSFDNLRRRNFRSSAGSGAAFHGISSLILRFPPNFQRQLSTKARRNCSNIGVAQIVAASWSNNSDNSPAAGAPAPPAATATDAATVPLPVVVAANEDVVVSAAADENGAVQLNSSSYSSFLKSDASKTIHAAERLGRGIETDGITTPVVNTSAYFFKKTADLIDFKENRQVSYEYGRYGNPTTVVLEEKISALEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDVLGGCISGSIKVVSQIRTLHHVLGGTLNPNAAYLFIRGMKTLHLRVQQQNSTGMRMAKLLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 4 - DNA, 5135 bp, Glycine max
  source 1..5135: mol_type = genomic DNA
agagtacagagagattcgttttggtaggtatatagatagaggagagagaaaccaaacacaaatccttgaaacgacgacggaaatggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcagcaccaaggcgcgccgcaattgtagcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccggtacgtaaaattcttgtcttttttttttgcgtcataaattgtgattcatgttttactttccaagattcgtttacttttgaactaaattggatgtgattttctcggataatgtttctttaagtgtactttttcaattcttttatgttttgttagatttttcattattcattacatttggatgtgatttgcttgagaaagagattttctcttgctttttatatgtactgtacataattaatcagtttttcctgcctgtctgacttggtatatttatatttattagtttatatgacgtttgatttaagatttaaagtaggtttcgtggttaatttgtatggattttaattcagttagttttaatatccttggatgttattgttgatggtaatgttgtttacgaagctgattgattttgaattctgaattttggcaatgcagctgaaagactgggtaggggtattgagactgatggaattaccacccctgtgctgaacacttctgcctacttttttaagaaaaccgctgatctcattgatttcaaggttggattgttaaaagatttattcgcctcatgacataatgagtggataaatgtcagttattggtgtggctaaatttttccatcatttggcaggagaatcgtcaagtgagttatgaatacgggcgctatggaaacccaacgactgtggttctggaggagaagataaggtttgtgtgttttgattttgttatttactattaacttgtaatgtgttttaagtgtggtggtcatttgatgaggtttgttgtcattattaaccccagtatattggagggggctgaatcaactgtgataatggcatctggaatgtgtgctagcgtagtcctatttatggcactggttccagctggtggacatcttgtgaccactacggattgttacaggaagactagaatattcattgagacttttcttccaaagatggggatcacggtaaatgttctactatcccattcttttggtggtttaattgtattttgtatttgttgcatggggcagaaactgcttcttatgttgtcacgtttctttagcttaaagagttatttaacttgtttggattttgaaagaatgcttattttcttatgattcaatgctatttggctacttaccaaatcatatcataagatgaatcttaaatgagtttgtatcttacggtacatgaatgaattatcattgatatgaatcacacattcaacatgtgattcattattttattattattttttaaacttttctttctccaattctcatgaaaaattgaatggtcataagtttaaattgttaaatgaaaaggagttgaaggagcacttcggaatttatcctatttgattatttcactactttttatccgtcacacagttattcaatgtatgtactcactactctatttacctatttaattgcttcatttttattcatctaaaataaagccttcacaaactcatatataactaataattttgttttattttttttatttaaaatttgaattttttacattgaacatgatatatttgaagtagtttttcctgttgtaagaatgcttattttctttcctgttgttcaaaattatatttgtgcatacataaattttattcaaattattaaaagaaatctcatggtgaataatattgccgagaatatggtttttggccgagtctaatagcgggcgatgtgtgatccacagagccaacctcgcctagtggaatgaggctattgatgttgttgtagtctttcctagaatataatgattaatagagacacctgatataaatgtcttgctgaaaatttgcttggttttcaactggaaatttgaaatggatatcttaaatgtgaatcttcttgtcagttgattgagtaaggacataaggtggacttttgacattttcaattttttatttctggtaggtttttactgttgtcctgggtgcttgcaatatatgtaacatattttccctttgatgaacagaccactgtaattgatccagcagatgttggagccttggaatctgcattggagcagcacaatgttagtgttggagaaagtagttggttttcttttatggatttctctgatttatttgtagttaatagatggtcttgagttaacatggtatgatacaatcattcaggtgtctctattcttcactgagtcgcctaccaatccattcctgagatgtgttgacattaagctggtttcagagctttgccacaagaaggggactttgctctgtattgatggtacatttgcaacaccattgaaccagaaggcccttgcccttggcgctgatctgattctgcactccttaacaaaatacatgggtggacatcatgatgtaagttggcatgttcaagatttagtttgatggaaaaacaacacaaatatgttacatttcagttgttcagagttaagcgtcaaggagcatttttcctctttcaggcccttggtggttgcataagtggttcaactaaggtggtttcgcaaattcggactttgcaccatgttttgggtggtacacttaacccggtaagtgctttctatagagctgagcctccatggaaggatatcgtaattggttattgtggataacatagtgccaatttttccgcagaatgctgcatacctattcatcagaggcatgaaaacgctgcatctccgtgtacagcagcagaattcaacaggaatgaggatggccgaacttttagaggcacatcccaaggtatcatggcattttgacatgcacactgtcatgcctcgtctccatgctatttaatataatttattattttgttaattgaaacttttaaagttgtcaaatgagattgtgtataattcaattgaattcaaatatataaaaaatgaaatgaatggttaacacatgacttgtttcttcatcgacagcttatttttgttgctgatctgtcttttaaactttttttcccctatgtatatttgtgtataatgatggttgaattggggttctacgcaaaaatgaagtgtatggttaaatgtcatatcttgttcatcaacagcatacttatgttggaatattgtgcactgctccctatgtactctaaaatgtatgtggttgtagcatgatggataatgtttactaagctgtgccaagagagcagatgctggtttcttttgaattttttttgttgtgatgctcttaaatgggagtcccttcatctgctattatcttgtatattaatgataataagtaattaagtattattacttttttgatagtagcttttaggatggaagcctaagtatttttgtggcataaaatttctcttgaaacaaaactgtattcttctctaggttcttcctttttttttccactttttgaattgtcaaatcagttgtttgtatcttttctcttctttttgatgtcattaacaaaggcatattcttataccattttatcattttacagattcagtatgtaaacattttcatgattgagtcctgtatctgatgtggctttaattacatactattattggcaggtgaagcgggtctactatccaggcttgccgagtcatcctgaacatgagcttgccaagaggcagatgactggtttcggtggtgttgtcagttttgaggtgggaatttgctaagttgcgattcaaatatgtggtttcctaataacacaattacttttttttttcttttatcaagaatgatgtattggataccatcatatggcatatttttgataattaaattgccaataatgcagattgatggagatctacataccacaataaagtttattgattcactgaaaatcccttatattgcggcctcgtttggtggctgtgagagcattgtggatcaacctgctattttgtcttactggtactcatttttattccatgttttaagttgtcatgttgatgatttgacactactcggggaatggtcctctacttgaatgtgaccctgtagtgaaagcatcttttgctggtccttttgttaatttttagatttggcttggtggtggttgttgtatgtttaatttaattttcaacatattgtgcaacagggatcttcctcagtcagaaagggccaagtataagatttatgacaacctggttcgcttcagctttggagttgaagattttgaggatttgaaggctgatgtcctgcaagctctggaagctatatagacagttttcctgattcacccatgttttttttcttttaataatcgtgttatatgttccttttcgttatcgcatctggcgattcaattgaattttgatcgtctaatgttctgttggaattgtgtttagatgaatggtttctaatttggatgttgtgaaacttgtgatgaatggttgaaattgaaacctctattgatattcattcccgcgtttcctacaaaaattaaaagaaaaatgaattcttgaggcaggatatataagagaatttctttttgaagcttttgtgatcatgttttctggtaaaaatgggggtgtttgtcctttatattttaccttggtccgttggagctgttttaattttgtttcaaagtagt

SEQ ID NO: 5 - DNA, 1608 bp, synthetic construct
  source 1..1608: mol_type = other DNA
atggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcagcaccaaggcgcgccgcaattgtagcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccgctgaaagactgggtaggggtattgagactgatggaattaccacccctgtgctgaacacttctgcctacttttttaagaaaaccgctgatctcattgatttcaaggagaatcgtcaagtgagttatgaatacgggcgctatggaaacccaacgactgtggttctggaggagaagataagtatattggagggggctgaatcaactgtgataatggcatctggaatgtgtgctagcgtagtcctatttatggcactggttccagctggtggacatcttgtgaccactacggattgttacaggaagactagaatattcattgagacttttcttccaaagatggggatcacgaccactgtaattgatccagcagatgttggagccttggaatctgcattggagcagcacaatgtgtctctattcttcactgagtcgcctaccaatccattcctgagatgtgttgacattaagctggtttcagagctttgccacaagaaggggactttgctctgtattgatggtacatttgcaacaccattgaaccagaaggcccttgcccttggcgctgatctgattctgcactccttaacaaaatacatgggtggacatcatgatgcccttggtggttgcataagtggttcaactaaggtggtttcgcaaattcggactttgcaccatgttttgggtggtacacttaacccgaatgctgcatacctattcatcagaggcatgaaaacgctgcatctccgtgtacagcagcagaattcaacaggaatgaggatggccgaacttttagaggcacatcccaaggtgaagcgggtctactatccaggcttgccgagtcatcctgaacatgagcttgccaagaggcagatgactggtttcggtggtgttgtcagttttgagattgatggagatctacataccacaataaagtttattgattcactgaaaatcccttatattgcggcctcgtttggtggctgtgagagcattgtggatcaacctgctattttgtcttactgggatcttcctcagtcagaaagggccaagtataagatttatgacaacctggttcgcttcagctttggagttgaagattttgaggatttgaaggctgatgtcctgcaagctctggaagctatatag

SEQ ID NO: 6 - protein, 535 aa, Glycine max
MAVSSSHMRFTFECRSDPDFSPPPPSFDNLRRRNFRSSAGSGAAFHGISSLILRFPPNFQRQLSTKARRNCSNIGVAQIVAASWSNNSDNSPAAGAPAPPAATAADAATVPLPVDLAAAEDVVVSAAAAENGTVQLNSSSYSFLKSDASKTIHAAERLGRGIETDGITTPVLNTSAYFFKKTADLIDFKENRQVSYEYGRYGNPTTVVLEEKISILEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDALGGCISGSTKVVSQIRTLHHVLGGTLNPNAAYLFIRGMKTLHLRVQQQNSTGMRMAELLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 7 - DNA, 3103 bp, Glycine max
  source 1..3103: mol_type = genomic DNA
agtggacccaaaagccatgcacaacaacacgtactcaccaaggtgcaatcgtgctgcccaaaaacattcaccaactcaatccatgatgagcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctagttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacag

SEQ ID NO: 8 - DNA, 1818 bp, synthetic construct
  source 1..1818: mol_type = other DNA
atgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacgggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgagagtttcttcctatctagcactgaagctcaacaatcctacttgcaaggattcagcaggaacattttagaggcctcctacgataccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctagttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactga

SEQ ID NO: 9 - protein, 605 aa, Glycine max
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERKQEEDEDEEQQRESEESEDSELRRHKNKNPFLFGSNRFETLFKNQYGRIRVLQRFNQRSPQLQNLRDYRILEFNSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGSQDNVISQIPSQVQELAFPGSAQAVEKLLKNQRESYFVDAQPKKKEEGNKGRKGPLSSILRAFY

SEQ ID NO: 10 - DNA, 3038 bp, Glycine max
  source 1..3038: mol_type = genomic DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctagttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 11 - DNA, 1818 bp, synthetic construct; identical to SEQ ID NO: 8
  source 1..1818: mol_type = other DNA

SEQ ID NO: 12 - protein, 605 aa, Glycine max; identical to SEQ ID NO: 9

SEQ ID NO: 13 - DNA, 2679 bp, Glycine max
  source 1..2679: mol_type = genomic DNA
aaccaccccaattttgttcaacaattcaacacacgtcactctgcatgcccatcctgtggccaaattttcatgcaagttaaccaaagtcgctttttctcttctgtaactatatatatatatatatatatatatatatatatagctctaatctcacaccgtgttttcatcatcacacaagagcgagcgcaacattattcgagtacgtagcttaacaatattttaagcaattgctgctacaatgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcggttagtacacttaactaattactactactaattaaacatcgatcatcactttgcttctgctcttagtactagttaatggtaatcaattttactaattacagggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggtactgcatgcctttttcattatgacagattagttaattaattcttctaattaatttatttccaaatgtgaagtgaatatcaatctttgtccttttctctttctctaggatttcttcctatctagcactcaagaccagcaatcctacttgcaaggcttcagcgagaatattctggaggcttccttcaatgtaagcaagcaacacatgcaccatctaatttgcatttccgtttagtggtttgtctaaatgtcatactaattaaatattatcattatatttgcttgtgtgcgcaattggttttatagaccaaattcgaggagataaacagggttttgtttggtggagaggggaggcggcaccagcaagagggagtgattttggaactatcaaaggaacagattcgggaactgagcaagcgtgccaaatctagttcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgaggtaagtgcaaaaaccatgtagatctagacaaagtgaactgcactattgagttctattcactctcttcttaattagttagttatactaattattgagactaaacaaatacagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagaggaacttccttgcaggtactatacatatataatattgattattgaataccctgttgttactttttgttgttttatggcatgtggaagcatgctgattgggttatttaattatttccaattcttggtgcgtgtgattgaaaatttgaaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttgaatatgtatgcttaggagactaggagttcacagcgagcgaggaaagtatagggatatgtaactagaataacttgagatccacctttctactactagtaaataaaataaataaatcgag

SEQ ID NO: 14 - DNA, 1755 bp, synthetic construct
  source 1..1755: mol_type = other DNA
atgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcgggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggatttcttcctatctagcactcaagaccagcaatcctacttgcaaggcttcagcgagaatattctggaggcttccttcaataccaaattcgaggagataaacagggttttgtttggtggagaggggaggcggcaccagcaagagggagtgattttggaactatcaaaggaacagattcgggaactgagcaagcgtgccaaatctagttcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagaggaacttccttgcaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttga

SEQ ID NO: 15 - protein, 584 aa, Glycine max
MIARFPLLPLLLLGIVFLASVSVSLQVKPPSHHECLQSCESETDPYKRKACKLSCKFVPEHGEQQHEEEEREREHPQPHPPHEERGRRERGRQEGEKEEKEQDERELQPRPFPRPEEHEGEQWKGRHRHEDPEERARMRLREAERIEREREEQRVREEEEKLRRREKKEQREEKEEEGQGSEDSHSKRQNNPFHFSSNRFQTLFKNQHGHLRVLQRFDQRSPQLENLRDYRVVELMAKPNTLFLPHHADADFLLLVLSGRALINLVKPDDRDPYYLDRGYAQRIPAGTTVYLVNPDKKKDLRVIKLAIPVNKPGNFEDFFLSSTQDQQSYLQGFSENILEASFNTKFEEINRVLFGGEGRRHQQEGVILELSKEQIRELSKRAKSSSRSTNSFDYEPFYLRGSQISSNNFGKFYEITPEKNPQLRDFDILLNTVDINEGGLLLPHYNSKAIVILMVTEGEANIELVGLKEQQQGEETREVRKYRAELSEDDIFVIPAAYPFVVNATSNLNFVAFGINAENNQRNFLAGSKDNVIRQIQKQVKELAFPAGSAQDIENLIKNQRESYFADAQPLQKEEGKKGSFVF

SEQ ID NO: 16 - DNA, 2787 bp, Glycine max
  source 1..2787: mol_type = genomic DNA
atcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagttcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgaggtaagcagaaaaactgaacatgacaattgagttctattcactttcttcttagttagagaaacctattcttgagactttaaataatgatttacttttttctttgttcacaaatatagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagaggaacttccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatat

SEQ ID NO: 17 - DNA, 1866 bp, synthetic construct
  source 1..1866: mol_type = other DNA
atgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacgggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgagagtttcttcctatctagcactcaagctcaacagtcctacttgcaagggttcagcaagaatattctagaggcctcatacgacaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagttcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagaggaacttccttgcaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactga

SEQ ID NO: 18 - protein, 621 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 19 - DNA, 5224 bp, Glycine max
  source 1..5224: mol_type = genomic DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaatggtgtttgttgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 20 - DNA, 1683 bp, synthetic construct
  source 1..1683: mol_type = other DNA
atgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaaggggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggaatttttcgggcctggaggacgagacccagaatcggtcctctcagcattcagctggaatgtgctgcaagctgcgctccaaaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaatggtgtttgttgtccctcccggtcatcccttcgtcaccatagcttccaataaagagaatctcctcatgatttgcttcgaggttaacgctcgagacaacaagaagtttacatttgcagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtga

SEQ ID NO: 21 - protein, 560 aa, Glycine max
MQLPPHSSLLPFPSLYIYIYIYIYIYIYIYIYIYKQAQCIISKKLREFFLFLTDQAMAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEARAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFSWNVLQAALQTPKGKLEKLFDQQNEGSIFAISREQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNYPSEMVNGVFDRKESFFFPFELPREERGRRADA

SEQ ID NO: 22 - DNA, 2725 bp, Glycine max
  source 1..2725: mol_type = genomic DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcatgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagaggaacttccttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 23 - DNA, 1320 bp, synthetic construct
  source 1..1320: mol_type = other DNA
atgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcgggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgatttcttcttatctagcactcaagcccaacagtcctacttgcaaggcttcagccataatattctagagacctccttccatagcgaattcgaggagataaacagggttttgtttggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaaggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagaggaacttccttgcaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactga

SEQ ID NO: 24 - protein, 439 aa, Glycine max
MMRVRFPLLVLLGTVFLASVCVSLKVREDENNPFYLRSSNSFQTLFENQNGRIRLLQRFNKRSPQLENLRDYRIVQFQSKPNTILLPHHADADFLLFVLSGRAILTLVNNDDRDSYNLHPGDAQRIPAGTTYYLVNPHDHQNLKIIKLAIPVNKPGRYDDFFLSSTQAQQSYLQGFSHNILETSFHSEFEEINRVLFGEEEEQRQQEGVIVELSKEQIRQLSRRAKSSSRKTISSEDEPFNLRSRNPIYSNNFGKFFEITPEKNPQLRDLDIFLSSVDINEGALLLPHFNSKAIVILVINEGDANIELVGIKEQQQKQKQEEEPLEVQRYRAELSEDDVFVIPAAYPFVVNATSNLNFLAFGINAENNQRNFLAGEKDNVVRQIERQVQELAFPGSAQDVERLLKKQRESYFVDAQPQQKEEGSKGRKGPFPSILGALY

SEQ ID NO: 25 - DNA, 2311 bp, Glycine max
  source 1..2311: mol_type = genomic DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcgtgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagaggaacttccttgcaggtactatacatacactgttactgctttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctccacctatcttctatctactacgaataaataaatccagcatatgatgcttgtgttttgtagccttcaactgttcaactgtcttcatatatatgcatggttggcacgtactcatatatatgctaaagtaatttacccgctcaagctaaattatctg

SEQ ID NO: 26 - DNA, 1320 bp, synthetic construct
  source 1..1320: mol_type = other DNA
atgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcgggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgatttcttcttatctagcactcaagcccaacagtcctacttgcaaggcttcagccataatattctagagacctccttccatagcgaattcgaggagataaacagggttttgttgggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaaggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagaggaacttccttgcaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactga

SEQ ID NO: 27 - protein, 439 aa, Glycine max
MMRVRFPLLVLLGTVFLASVCVSLKVREDENNPFYLRSSNSFQTLFENQNGRIRLLQRFNKRSPQLENLRDYRIVQFQSKPNTILLPHHADADFLLFVLSGRAILTLVNNDDRDSYNLHPGDAQRIPAGTTYYLVNPHDHQNLKIIKLAIPVNKPGRYDDFFLSSTQAQQSYLQGFSHNILETSFHSEFEEINRVLLGEEEEQRQQEGVIVELSKEQIRQLSRRAKSSSRKTISSEDEPFNLRSRNPIYSNNFGKFFEITPEKNPQLRDLDIFLSSVDINEGALLLPHFNSKAIVILVINEGDANIELVGIKEQQQKQKQEEEPLEVQRYRAELSEDDVFVIPAAYPFVVNATSNLNFLAFGINAENNQRNFLAGEKDNVVRQIERQVQELAFPGSAQDVERLLKKQRESYFVDAQPQQKEEGSKGRKGPFPSILGALY

SEQ ID NO: 28 - DNA, 4137 bp, Glycine max
  source 1..4137: mol_type = genomic DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaatggtgtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 29 - DNA, 1521 bp, synthetic construct
  source 1..1521: mol_type = other DNA
atggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaaggggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggaatttttcgggcctggaggacgagacccagaatcggtcctctcagcattcagctggaatgtgctgcaagctgcgctccaaaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaatggtgtttgttgtccctcctggtcatcccttcgtcactatagcttccaataaagagaatctcctcataatttgcttcgaggttaacgttcgagacaacaagaagtttacgtttgcagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtga

SEQ ID NO: 30 - protein, 506 aa, Glycine max
MDHLKPRVSLLFLTDLTMATRAKLSLAIFLFFLLALISNLALGKLKETEVEEDPELVTCKHQCQQQRQYTESDKRTCLQQCDSMKQEREKQVEEETREKEEEHQEQHEEEEDENPYVFEEDKDFSTRVETEGGSIRVLKKFTEKSKLLQGIENFRLAILEARAHTFVSPRHFDSEVVLFNIKGRAVLGLVRESETEKITLEPGDMIHIPAGTPLYIVNRDENEKLLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNSHATKIALVMDGRGHLQISCPHMSSRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 31 - DNA, 63 bp, Arabidopsis thaliana
  source 1..63: mol_type = genomic DNA
agaaactgtagcaacatcggtgttgcacagatcgtggcggctaagtggtccaacaacccatcc

SEQ ID NO: 32 - protein, 21 aa, Arabidopsis thaliana
RNCSNIGVAQIVAAKWSNNPS

SEQ ID NO: 33 - DNA, 63 bp, synthetic construct
  source 1..63: mol_type = other DNA
agaaactgtagcaacatcagtgttgcacagatcgtggcggctaagtggtccaacaacccatcc

SEQ ID NO: 34 - protein, 21 aa, synthetic construct
RNCSNISVAQIVAAKWSNNPS

SEQ ID NO: 35 - DNA, 24 bp, synthetic construct
  source 1..24: mol_type = other DNA
agaaactgtagcaacaacccatcc

SEQ ID NO: 36 - DNA, 21 bp, synthetic construct
  source 1..21: mol_type = other DNA
agaaactgtagcaacccatcc

SEQ ID NO: 37 - DNA, 50 bp, Glycine max
  source 1..50: mol_type = genomic DNA
agcaccaaggcgcgccgcaactgcagcaacatcggcgtcgcgcaaatcgt

SEQ ID NO: 38 - DNA, 50 bp, Glycine max
  source 1..50: mol_type = genomic DNA
agcaccaaggcgcgccgcaattgtagcaacatcggcgtcgcgcaaattgt

SEQ ID NO: 39 - DNA, 22 bp, synthetic construct
  source 1..22: mol_type = other DNA
acgtgccaaatctagttcaagg

SEQ ID NO: 40 - DNA, 22 bp, synthetic construct
  source 1..22: mol_type = other DNA
ccagaggaacttccttgcaggt

SEQ ID NO: 41 - DNA, 22 bp, synthetic construct
  source 1..22: mol_type = other DNA
catgcaacgaagatagcactgg

SEQ ID NO: 42 - DNA, 22 bp, synthetic construct
  source 1..22: mol_type = other DNA
cctggaatggtgtttgttgtcc

SEQ ID NO: 43 - DNA, 38 bp, Glycine max
  source 1..38: mol_type = genomic DNA
cagctaagcaccaaggcgcgccgcaactgcagcaacat

SEQ ID NO: 44 - DNA, 37 bp, synthetic construct
  source 1..37: mol_type = other DNA
cagctaagcaccaagggcgccgcaactgcagcaacat

SEQ ID NO: 45 - DNA, 34 bp, synthetic construct
  source 1..34: mol_type = other DNA
cagctaagcaccaaggccgcaactgcagcaacat

SEQ ID NO: 46 - DNA, 35 bp, synthetic construct
  source 1..35: mol_type = other DNA
cagctaagcaccaagcgccgcaactgcagcaacat

SEQ ID NO: 47 - DNA, 38 bp, Glycine max
  source 1..38: mol_type = genomic DNA
cagctcagcaccaaggcgcgccgcaattgtagcaacat

SEQ ID NO: 48 - DNA, 11 bp, synthetic construct
  source 1..11: mol_type = other DNA
cagctcaacat

SEQ ID NO: 49 - DNA, 26 bp, synthetic construct
  source 1..26: mol_type = other DNA
cagctcagcaccaattgtagcaacat

SEQ ID NO: 50 - DNA, 40 bp, synthetic construct
  source 1..40: mol_type = other DNA
cagctcagcaccaaggtcgcgccgcaattggtagcaacat

SEQ ID NO: 51 - DNA, 39 bp, synthetic construct
  source 1..39: mol_type = other DNA
cagctcagcaccaaggtcgcgccgcaattgtagcaacat

SEQ ID NO: 52 - DNA, 4990 bp, synthetic construct
  source 1..4990: mol_type = other DNA
aaatggaagagtagcgatagacagaggagagagaaagagagggagtaagtgggtaaaggaaagaaaactaaagaaacaaacctaacacaacacaaatccttgaaacgacgacggaaatggccgtttcgagctcgcacatgcgtttcacctttgagtgccgctccgatcccgatttctcgccccccccgccgtccttcgacaacctccgccgccgaaacttccgctcctccgcaggatccggcgcggcgtttcacggcatctcctccctcatcctccgcttccctcccaacttccagcgccagctaagcaccaagggcgccgcaactgcagcaacatcggcgtcgcgcaaatcgtcgccgcttcgtggtcgaacaacagcgacaactctccggccgccggggctccggcgccgcccgcggccaccgccacggacgccgctacggtgcctctccccgtcgtcgtcgccgccaacgaggacgtcgttgtctccgccgcggcagacgagaacggggctgtacagttaaacagtagttcttattcttcatttttgaaatccgatgcaagcaaaacgattcatgccggtacgtgaaattcttgtctttttttgcgtcattaattgttattcaagttttactttccaaaattcgattactttttaattaaattagatgtgatttccttggataatgattctttaaattgtaccttttcaatctctttatgttttgtttgatttttcattaatcattacatttggatgtgatttgcttaagaaagagattttctcttgctttttgtttgtactgtacagaattaaatactttttcttgtcttactcggtatatttattagtttatatgaagtttgatttaggatttagattagggtttttggttaatttgtatggattttaatgcagttagttttaatttggattgattttgaattcgggcaatgcagctgaaagactgggtaggggtattgagactgatggaattaccacccctgtggttaacacttctgcctacttttttaagaaaaccgctgatctcattgatttcaaggtactgttctttggactgttattaaagcttttattcgcctaatgcgacaaaggtaaatgccagttattggtgtggctaaatttctcatcatttggcaggagaatcgtcaagtgagttatgaatacgggcgctatggaaacccaacgacggtggttctggaggagaagataaggttcgtgtgttttgattttgttattttaattatcttgtaatgtgttttaggtgttggggtcatttgatgaggtttgttgtcattatttaccagtgcattggagggggccgaatcaactgtgataatggcgtctgggatgtgtgctagcgtagtcctgtttatggcactggttccagctggtggacatcttgtgaccactacggattgttataggaagactagaatattcattgagacttttcttccaaagatggggatcacggtaaatgttcttctaatatattcatttggtggtttaattgtattttgtatttgttgcatggaaactgcttcatatgttgtcacgtttctctagcttaagagttgtttaacctgtttggattcaaagaatgcttattttcttatgattcaatgcaatttggctacttaccaaatcatatcataagatgaatcttaaatgagtttgtatcttgcggtacataaatgaatccgtgtaattttatatcatcgatatgagtcacacattcattgtgtgattcatttttatttttattattatttaaacttttctttcaccaattattgtgaaaaaattgaatagtcatactagtttaaatagttaaatgaaaaggaattgaaggaacacttctgaatttatcctatttgattattcaactactttttatctttcacacaattgttcaatgcatgcactctatttacctatttaattgctttgtttctattcttcttaaataaacttcacaaactcatatataactaatattttttggtatgatatatttgaagtagtttttcttgttgaaagaatgcttcttctttttctgttgttcaaaattctttgtgcatgcataattttactcaaattattaaaagaaatctcatggtgaataatattactgagaatatggtttttggtcaagtctaatagcaggcgatgtgtgatccatagagccaacctcacctactggaatgaagttattgatgttgttgtagtctttcctagaatataatgattactattagaggcacctaatataaatgtctcactgaaaatttgcttggttttcaactgaaaaattgaaatggatatcttaaatgcgaatctacttgtcagttgatagaataaggacataaggtggacttttgacattttcaatgtgtttatttctggtaggtttttactgttgtcttgggcccttgcaattataaatttataattgttatagatttgtcatatatatgtaacatactttccctttgatgaacagaccactgtaattgatccagcagatgttggagccttggaatctgcattggagcagcacaatgttagtgttggagagagtagttggttttcttttatggaattctctgattgatttatttgtagttaatagatggtctagagttaatatggtattatacaatcgtgcaggtgtctctattcttcactgagtctcctaccaatccattcctgagatgtgttgatattaagctggtttcagagctttgccacaagaaggggactttgctctgtattgatggtacatttgcaactccattgaaccagaaggcccttgcccttggcgctgatctgattctgcactccttaacaaaatacatgggtggacatcatgatgtaagtcagcctgttctttaaaatttagtttgatggaaacaacacaaattttactatttcagtagttcatagttaagcatcaaggaatgtttttcctctttcaggtccttggtggttgcataagtggttcaattaaggtggtttcgcaaattcggactttgcaccatgttttgggtggtacacttaacccggtaagtgatttctatagggccgagcctccatggaaggatatcttaatttgttattgtggctaacatggtgccaatttctttgcagaatgctgcatacctattcatcagaggcatgaaaacgctgcatctccgtgtacagcagcagaattcaacaggaatgaggatggccaaacttttagaggcacatcccaaggtatcatggcattgatatgcacactgtctttcctcctctccatgctatttaatataatttattatttggttaattgaaatatttaaagctgttgaatgagcatgtgtgtataagtatgtttcaattgcattcaattatataaaatgaatggttaacatatgacttgtgtataatgatggttgaattgtgttctatgtaaaatgaagtatatgtttaatatgccatttcctcttgttcatcaacaacatatttatgttgaaaaatcgtgtattaccccccatatgtacactaaatgtatgtggttataggatgatggacaatgtgtactaagctgtgccaagagtgcaggtgctggtttcttttgaattttttatgttgtgagttgtgatgcacttaaatgtgcatcccttgatctgctattatcttgtatgttaatgatagtaagtaattaagtattattacttttttgatagtagcttttaggatggatgctaagtatgtctgtggcataatttctcttgaaacaaaactgtattcttctctaggttcttcctttttttctttcttttctttttcctctttttgaattatcaaatcagttgtttgtatcttttctttttctgtttggacaccattattatctaatgcatatttttataccattttatcattttacagattcaatatgtaaatattttcatgattgagtcctgtatctgatgtgacttggctttaattacatactactattggcaggtgaagcgggtctactatccaggcttgccgagtcaccctgaacatgagcttgccaagaggcagatgactggtttcggtggtgttgtcagttttgaggtgggaatttgacaagttgctattcaaacatgtggtttcctaataacactattttttttttcttttatcaagaatggtgtattggataacatcatgtgccatattttttttatagttaaattgccaatattgcagattgatggagatctacataccacaataaaatttattgattcattgaaaatcccatatattgcggcctcgtttggtggctgtgagagcattgtggatcaacctgctattttgtcttactggtactcatttggattccatgttcaatttgtcatgttgatgattttgacactacacagggaatggtcctctacttgaatgtgaccctgtggtgaaagcatctttagctggtccttttggtttgttatagtgtcaaagtttaccttgtttttgttaatttaatttaattttcaacatattgtgcaacagggatcttcctcagtcagaaagggccaagtacaagatttatgacaacctggttcgcttcagctttggagttgaagattttgaggatttgaaggctgatgtcctgcaagctctggaagctatatagacagttttcctgattcacccaagtttttttcttttataattgtgctatttgtttgttatcacatctggcgattcaattgaattttgatcgtctaatgttctgttggaattgtgttaagatgaatggtctctaatttggatgttatgaaacttgtgatgaattgttgaaattgaaacctctatttgatgatattcttacccgtttttcctacaaatatcaaagcaggatatctgagaattggaaaatgataaatggtatgaaagcagtttcgtgtgaaatttgcacgaatg

SEQ ID NO: 53 - DNA, 4987 bp, synthetic construct
  source 1..4987: mol_type = other DNA
aaatggaagagtagcgatagacagaggagagagaaagagagggagtaagtgggtaaaggaaagaaaactaaagaaacaaacctaacacaacacaaatccttgaaacgacgacggaaatggccgtttcgagctcgcacatgcgtttcacctttgagtgccgctccgatcccgatttctcgccccccccgccgtccttcgacaacctccgccgccgaaacttccgctcctccgcaggatccggcgcggcgtttcacggcatctcctccctcatcctccgcttccctcccaacttccagcgccagctaagcaccaaggccgcaactgcagcaacatcggcgtcgcgcaaatcgtcgccgcttcgtggtcgaacaacagcgacaactctccggccgccggggctccggcgccgcccgcggccaccgccacggacgccgctacggtgcctctccccgtcgtcgtcgccgccaacgaggacgtcgttgtctccgccgcggcagacgagaacggggctgtacagttaaacagtagttcttattcttcatttttgaaatccgatgcaagcaaaacgattcatgccggtacgtgaaattcttgtctttttttgcgtcattaattgttattcaagttttactttccaaaattcgattactttttaattaaattagatgtgatttccttggataatgattctttaaattgtaccttttcaatctctttatgttttgtttgatttttcattaatcattacatttggatgtgatttgcttaagaaagagattttctcttgctttttgtttgtactgtacagaattaaatactttttcttgtcttactcggtatatttattagtttatatgaagtttgatttaggatttagattagggtttttggttaatttgtatggattttaatgcagttagttttaatttggattgattttgaattcgggcaatgcagctgaaagactgggtaggggtattgagactgatggaattaccacccctgtggttaacacttctgcctacttttttaagaaaaccgctgatctcattgatttcaaggtactgttctttggactgttattaaagcttttattcgcctaatgcgacaaaggtaaatgccagttattggtgtggctaaatttctcatcatttggcaggagaatcgtcaagtgagttatgaatacgggcgctatggaaacccaacgacggtggttctggaggagaagataaggttcgtgtgttttgattttgttattttaattatcttgtaatgtgttttaggtgttggggtcatttgatgaggtttgttgtcattatttaccagtgcattggagggggccgaatcaactgtgataatggcgtctgggatgtgtgctagcgtagtcctgtttatggcactggttccagctggtggacatcttgtgaccactacggattgttataggaagactagaatattcattgagacttttcttccaaagatggggatcacggtaaatgttcttctaatatattcatttggtggtttaattgtattttgtatttgttgcatggaaactgcttcatatgttgtcacgtttctctagcttaagagttgtttaacctgtttggattcaaagaatgcttattttcttatgattcaatgcaatttggctacttaccaaatcatatcataagatgaatcttaaatgagtttgtatcttgcggtacataaatgaatccgtgtaattttatatcatcgatatgagtcacacattcattgtgtgattcatttttatttttattattatttaaacttttctttcaccaattattgtgaaaaaattgaatagtcatactagtttaaatagttaaatgaaaaggaattgaaggaacacttctgaatttatcctatttgattattcaactactttttatctttcacacaattgttcaatgcatgcactctatttacctatttaattgctttgtttctattcttcttaaataaacttcacaaactcatatataactaatattttttggtatgatatatttgaagtagtttttcttgttgaaagaatgcttcttctttttctgttgttcaaaattctttgtgcatgcataattttactcaaattattaaaagaaatctcatggtgaataatattactgagaatatggtttttggtcaagtctaatagcaggcgatgtgtgatccatagagccaacctcacctactggaatgaagttattgatgttgttgtagtctttcctagaatataatgattactattagaggcacctaatataaatgtctcactgaaaatttgcttggttttcaactgaaaaattgaaatggatatcttaaatgcgaatctacttgtcagttgatagaataaggacataaggtggacttttgacattttcaatgtgtttatttctggtaggtttttactgttgtcttgggcccttgcaattataaatttataattgttatagatttgtcatatatatgtaacatactttccctttgatgaacagaccactgtaattgatccagcagatgttggagccttggaatctgcattggagcagcacaatgttagtgttggagagagtagttggttttcttttatggaattctctgattgatttatttgtagttaatagatggtctagagttaatatggtattatacaatcgtgcaggtgtctctattcttcactgagtctcctaccaatccattcctgagatgtgttgatattaagctggtttcagagctttgccacaagaaggggactttgctctgtattgatggtacatttgcaactccattgaaccagaaggcccttgcccttggcgctgatctgattctgcactccttaacaaaatacatgggtggacatcatgatgtaagtcagcctgttctttaaaatttagtttgatggaaacaacacaaattttactatttcagtagttcatagttaagcatcaaggaatgtttttcctctttcaggtccttggtggttgcataagtggttcaattaaggtggtttcgcaaattcggactttgcaccatgttttgggtggtacacttaacccggtaagtgatttctatagggccgagcctccatggaaggatatcttaatttgttattgtggctaacatggtgccaatttctttgcagaatgctgcatacctattcatcagaggcatgaaaacgctgcatctccgtgtacagcagcagaattcaacaggaatgaggatggccaaacttttagaggcacatcccaaggtatcatggcattgatatgcacactgtctttcctcctctccatgctatttaatataatttattatttggttaattgaaatatttaaagctgttgaatgagcatgtgtgtataagtatgtttcaattgcattcaattatataaaatgaatggttaacatatgacttgtgtataatgatggttgaattgtgttctatgtaaaatgaagtatatgtttaatatgccatttcctcttgttcatcaacaacatatttatgttgaaaaatcgtgtattaccccccatatgtacactaaatgtatgtggttataggatgatggacaatgtgtactaagctgtgccaagagtgcaggtgctggtttcttttgaattttttatgttgtgagttgtgatgcacttaaatgtgcatcccttgatctgctattatcttgtatgttaatgatagtaagtaattaagtattattacttttttgatagtagcttttaggatggatgctaagtatgtctgtggcataatttctcttgaaacaaaactgtattcttctctaggttcttcctttttttctttcttttctttttcctctttttgaattatcaaatcagttgtttgtatcttttctttttctgtttggacaccattattatctaatgcatatttttataccattttatcattttacagattcaatatgtaaatattttcatgattgagtcctgtatctgatgtgacttggctttaattacatactactattggcaggtgaagcgggtctactatccaggcttgccgagtcaccctgaacatgagcttgccaagaggcagatgactggtttcggtggtgttgtcagttttgaggtgggaatttgacaagttgctattcaaacatgtggtttcctaataacactattttttttttcttttatcaagaatggtgtattggataacatcatgtgccatattttttttatagttaaattgccaatattgcagattgatggagatctacataccacaataaaatttattgattcattgaaaatcccatatattgcggcctcgtttggtggctgtgagagcattgtggatcaacctgctattttgtcttactggtactcatttggattccatgttcaatttgtcatgttgatgattttgacactacacagggaatggtcctctacttgaatgtgaccctgtggtgaaagcatctttagctggtccttttggtttgttatagtgtcaaagtttaccttgtttttgttaatttaatttaattttcaacatattgtgcaacagggatcttcctcagtcagaaagggccaagtacaagatttatgacaacctggttcgcttcagctttggagttgaagattttgaggatttgaaggctgatgtcctgcaagctctggaagctatatagacagttttcctgattcacccaagtttttttcttttataattgtgctatttgtttgttatcacatctggcgattcaattgaattttgatcgtctaatgttctgttggaattgtgttaagatgaatggtctctaatttggatgttatgaaacttgtgatgaattgttgaaattgaaacctctatttgatgatattcttacccgtttttcctacaaatatcaaagcaggatatctgagaattggaaaatgataaatggtatgaaagcagtttcgtgtgaaatttgcacgaatg

SEQ ID NO: 54 - DNA, 4988 bp, synthetic construct
  source 1..4988: mol_type = other DNA
aaatggaagagtagcgatagacagaggagagagaaagagagggagtaagtgggtaaaggaaagaaaactaaagaaacaaacctaacacaacacaaatccttgaaacgacgacggaaatggccgtttcgagctcgcacatgcgtttcacctttgagtgccgctccgatcccgatttctcgccccccccgccgtccttcgacaacctccgccgccgaaacttccgctcctccgcaggatccggcgcggcgtttcacggcatctcctccctcatcctccgcttccctcccaacttccagcgccagctaagcaccaagcgccgcaactgcagcaacatcggcgtcgcgcaaatcgtcgccgcttcgtggtcgaacaacagcgacaactctccggccgccggggctccggcgccgcccgcggccaccgccacggacgccgctacggtgcctctccccgtcgtcgtcgccgccaacgaggacgtcgttgtctccgccgcggcagacgagaacggggctgtacagttaaacagtagttcttattcttcatttttgaaatccgatgcaagcaaaacgattcatgccggtacgtgaaattcttgtctttttttgcgtcattaattgttattcaagttttactttccaaaattcgattactttttaattaaattagatgtgatttccttggataatgattctttaaattgtaccttttcaatctctttatgttttgtttgatttttcattaatcattacatttggatgtgatttgcttaagaaagagattttctcttgctttttgtttgtactgtacagaattaaatactttttcttgtcttactcggtatatttattagtttatatgaagtttgatttaggatttagattagggtttttggttaatttgtatggattttaatgcagttagttttaatttggattgattttgaattcgggcaatgcagctgaaagactgggtaggggtattgagactgatggaattaccacccctgtggttaacacttctgcctacttttttaagaaaaccgctgatctcattgatttcaaggtactgttctttggactgttattaaagcttttattcgcctaatgcgacaaaggtaaatgccagttattggtgtggctaaatttctcatcatttggcaggagaatcgtcaagtgagttatgaatacgggcgctatggaaacccaacgacggtggttctggaggagaagataaggttcgtgtgttttgattttgttattttaattatcttgtaatgtgttttaggtgttggggtcatttgatgaggtttgttgtcattatttaccagtgcattggagggggccgaatcaactgtgataatggcgtctgggatgtgtgctagcgtagtcctgtttatggcactggttccagctggtggacatcttgtgaccactacggattgttataggaagactagaatattcattgagacttttcttccaaagatggggatcacggtaaatgttcttctaatatattcatttggtggtttaattgtattttgtatttgttgcatggaaactgcttcatatgttgtcacgtttctctagcttaagagttgtttaacctgtttggattcaaagaatgcttattttcttatgattcaatgcaatttggctacttaccaaatcatatcataagatgaatcttaaatgagtttgtatcttgcggtacataaatgaatccgtgtaattttatatcatcgatatgagtcacacattcattgtgtgattcatttttatttttattattatttaaacttttctttcaccaattattgtgaaaaaattgaatagtcatactagtttaaatagttaaatgaaaaggaattgaaggaacacttctgaatttatcctatttgattattcaactactttttatctttcacacaattgttcaatgcatgcactctatttacctatttaattgctttgtttctattcttcttaaataaacttcacaaactcatatataactaatattttttggtatgatatatttgaagtagtttttcttgttgaaagaatgcttcttctttttctgttgttcaaaattctttgtgcatgcataattttactcaaattattaaaagaaatctcatggtgaataatattactgagaatatggtttttggtcaagtctaatagcaggcgatgtgtgatccatagagccaacctcacctactggaatgaagttattgatgttgttgtagtctttcctagaatataatgattactattagaggcacctaatataaatgtctcactgaaaatttgcttggttttcaactgaaaaattgaaatggatatcttaaatgcgaatctacttgtcagttgatagaataaggacataaggtggacttttgacattttcaatgtgtttatttctggtaggtttttactgttgtcttgggcccttgcaattataaatttataattgttatagatttgtcatatatatgtaacatactttccctttgatgaacagaccactgtaattgatccagcagatgttggagccttggaatctgcattggagcagcacaatgttagtgttggagagagtagttggttttcttttatggaattctctgattgatttatttgtagttaatagatggtctagagttaatatggtattatacaatcgtgcaggtgtctctattcttcactgagtctcctaccaatccattcctgagatgtgttgatattaagctggtttcagagctttgccacaagaaggggactttgctctgtattgatggtacatttgcaactccattgaaccagaaggcccttgcccttggcgctgatctgattctgcactccttaacaaaatacatgggtggacatcatgatgtaagtcagcctgttctttaaaatttagtttgatggaaacaacacaaattttactatttcagtagttcatagttaagcatcaaggaatgtttttcctctttcaggtccttggtggttgcataagtggttcaattaaggtggtttcgcaaattcggactttgcaccatgttttgggtggtacacttaacccggtaagtgatttctatagggccgagcctccatggaaggatatcttaatttgttattgtggctaacatggtgccaatttctttgcagaatgctgcatacctattcatcagaggcatgaaaacgctgcatctccgtgtacagcagcagaattcaacaggaatgaggatggccaaacttttagaggcacatcccaaggtatcatggcattgatatgcacactgtctttcctcctctccatgctatttaatataatttattatttggttaattgaaatatttaaagctgttgaatgagcatgtgtgtataagtatgtttcaattgcattcaattatataaaatgaatggttaacatatgacttgtgtataatgatggttgaattgtgttctatgtaaaatgaagtatatgtttaatatgccatttcctcttgttcatcaacaacatatttatgttgaaaaatcgtgtattaccccccatatgtacactaaatgtatgtggttataggatgatggacaatgtgtactaagctgtgccaagagtgcaggtgctggtttcttttgaattttttatgttgtgagttgtgatgcacttaaatgtgcatcccttgatctgctattatcttgtatgttaatgatagtaagtaattaagtattattacttttttgatagtagcttttaggatggatgctaagtatgtctgtggcataatttctcttgaaacaaaactgtattcttctctaggttcttcctttttttctttcttttctttttcctctttttgaattatcaaatcagttgtttgtatcttttctttttctgtttggacaccattattatctaatgcatatttttataccattttatcattttacagattcaatatgtaaatattttcatgattgagtcctgtatctgatgtgacttggctttaattacatactactattggcaggtgaagcgggtctactatccaggcttgccgagtcaccctgaacatgagcttgccaagaggcagatgactggtttcggtggtgttgtcagttttgaggtgggaatttgacaagttgctattcaaacatgtggtttcctaataacactattttttttttcttttatcaagaatggtgtattggataacatcatgtgccatattttttttatagttaaattgccaatattgcagattgatggagatctacataccacaataaaatttattgattcattgaaaatcccatatattgcggcctcgtttggtggctgtgagagcattgtggatcaacctgctattttgtcttactggtactcatttggattccatgttcaatttgtcatgttgatgattttgacactacacagggaatggtcctctacttgaatgtgaccctgtggtgaaagcatctttagctggtccttttggtttgttatagtgtcaaagtttaccttgtttttgttaatttaatttaattttcaacatattgtgcaacagggatcttcctcagtcagaaagggccaagtacaagatttatgacaacctggttcgcttcagctttggagttgaagattttgaggatttgaaggctgatgtcctgcaagctctggaagctatatagacagttttcctgattcacccaagtttttttcttttataattgtgctatttgtttgttatcacatctggcgattcaattgaattttgatcgtctaatgttctgttggaattgtgttaagatgaatggtctctaatttggatgttatgaaacttgtgatgaattgttgaaattgaaacctctatttgatgatattcttacccgtttttcctacaaatatcaaagcaggatatctgagaattggaaaatgataaatggtatgaaagcagtttcgtgtgaaatttgcacgaatg

SEQ ID NO: 55 - protein, 535 aa, synthetic construct
MAVSSSHMRFTFECRSDPDFSPPPPSFDNLRRRNFRSSAGSGAAFHGISSLILRFPPNFQRQLSTKRRNCSNIGVAQIVAASWSNNSDNSPAAGAPAPPAATATDAATVPLPVVVAANEDVVVSAAADENGAVQLNSSSYSSFLKSDASKTIHAAERLGRGIETDGITTPVVNTSAYFFKKTADLIDFKENRQVSYEYGRYGNPTTVVLEEKISALEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDVLGGCISGSIKVVSQIRTLHHVLGGTLNPNAAYLFIRGMKTLHLRVQQQNSTGMRMAKLLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 56 - DNA, 5108 bp, synthetic construct
  source 1..5108: mol_type = other DNA
agagtacagagagattcgttttggtaggtatatagatagaggagagagaaaccaaacacaaatccttgaaacgacgacggaaatggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccggtacgtaaaattcttgtcttttttttttgcgtcataaattgtgattcatgttttactttccaagattcgtttacttttgaactaaattggatgtgattttctcggataatgtttctttaagtgtactttttcaattcttttatgttttgttagatttttcattattcattacatttggatgtgatttgcttgagaaagagattttctcttgctttttatatgtactgtacataattaatcagtttttcctgcctgtctgacttggtatatttatatttattagtttatatgacgtttgatttaagatttaaagtaggtttcgtggttaatttgtatggattttaattcagttagttttaatatccttggatgttattgttgatggtaatgttgtttacgaagctgattgattttgaattctgaattttggcaatgcagctgaaagactgggtaggggtattgagactgatggaattaccacccctgtgctgaacacttctgcctacttttttaagaaaaccgctgatctcattgatttcaaggttggattgttaaaagatttattcgcctcatgacataatgagtggataaatgtcagttattggtgtggctaaatttttccatcatttggcaggagaatcgtcaagtgagttatgaatacgggcgctatggaaacccaacgactgtggttctggaggagaagataaggtttgtgtgttttgattttgttatttactattaacttgtaatgtgttttaagtgtggtggtcatttgatgaggtttgttgtcattattaaccccagtatattggagggggctgaatcaactgtgataatggcatctggaatgtgtgctagcgtagtcctatttatggcactggttccagctggtggacatcttgtgaccactacggattgttacaggaagactagaatattcattgagacttttcttccaaagatggggatcacggtaaatgttctactatcccattcttttggtggtttaattgtattttgtatttgttgcatggggcagaaactgcttcttatgttgtcacgtttctttagcttaaagagttatttaacttgtttggattttgaaagaatgcttattttcttatgattcaatgctatttggctacttaccaaatcatatcataagatgaatcttaaatgagtttgtatcttacggtacatgaatgaattatcattgatatgaatcacacattcaacatgtgattcattattttattattattttttaaacttttctttctccaattctcatgaaaaattgaatggtcataagtttaaattgttaaatgaaaaggagttgaaggagcacttcggaatttatcctatttgattatttcactactttttatccgtcacacagttattcaatgtatgtactcactactctatttacctatttaattgcttcatttttattcatctaaaataaagccttcacaaactcatatataactaataattttgttttattttttttatttaaaatttgaattttttacattgaacatgatatatttgaagtagtttttcctgttgtaagaatgcttattttctttcctgttgttcaaaattatatttgtgcatacataaattttattcaaattattaaaagaaatctcatggtgaataatattgccgagaatatggtttttggccgagtctaatagcgggcgatgtgtgatccacagagccaacctcgcctagtggaatgaggctattgatgttgttgtagtctttcctagaatataatgattaatagagacacctgatataaatgtcttgctgaaaatttgcttggttttcaactggaaatttgaaatggatatcttaaatgtgaatcttcttgtcagttgattgagtaaggacataaggtggacttttgacattttcaattttttatttctggtaggtttttactgttgtcctgggtgcttgcaatatatgtaacatattttccctttgatgaacagaccactgtaattgatccagcagatgttggagccttggaatctgcattggagcagcacaatgttagtgttggagaaagtagttggttttcttttatggatttctctgatttatttgtagttaatagatggtcttgagttaacatggtatgatacaatcattcaggtgtctctattcttcactgagtcgcctaccaatccattcctgagatgtgttgacattaagctggtttcagagctttgccacaagaaggggactttgctctgtattgatggtacatttgcaacaccattgaaccagaaggcccttgcccttggcgctgatctgattctgcactccttaacaaaatacatgggtggacatcatgatgtaagttggcatgttcaagatttagtttgatggaaaaacaacacaaatatgttacatttcagttgttcagagttaagcgtcaaggagcatttttcctctttcaggcccttggtggttgcataagtggttcaactaaggtggtttcgcaaattcggactttgcaccatgttttgggtggtacacttaacccggtaagtgctttctatagagctgagcctccatggaaggatatcgtaattggttattgtggataacatagtgccaatttttccgcagaatgctgcatacctattcatcagaggcatgaaaacgctgcatctccgtgtacagcagcagaattcaacaggaatgaggatggccgaacttttagaggcacatcccaaggtatcatggcattttgacatgcacactgtcatgcctcgtctccatgctatttaatataatttattattttgttaattgaaacttttaaagttgtcaaatgagattgtgtataattcaattgaattcaaatatataaaaaatgaaatgaatggttaacacatgacttgtttcttcatcgacagcttatttttgttgctgatctgtcttttaaactttttttcccctatgtatatttgtgtataatgatggttgaattggggttctacgcaaaaatgaagtgtatggttaaatgtcatatcttgttcatcaacagcatacttatgttggaatattgtgcactgctccctatgtactctaaaatgtatgtggttgtagcatgatggataatgtttactaagctgtgccaagagagcagatgctggtttcttttgaattttttttgttgtgatgctcttaaatgggagtcccttcatctgctattatcttgtatattaatgataataagtaattaagtattattacttttttgatagtagcttttaggatggaagcctaagtatttttgtggcataaaatttctcttgaaacaaaactgtattcttctctaggttcttcctttttttttccactttttgaattgtcaaatcagttgtttgtatcttttctcttctttttgatgtcattaacaaaggcatattcttataccattttatcattttacagattcagtatgtaaacattttcatgattgagtcctgtatctgatgtggctttaattacatactattattggcaggtgaagcgggtctactatccaggcttgccgagtcatcctgaacatgagcttgccaagaggcagatgactggtttcggtggtgttgtcagttttgaggtgggaatttgctaagttgcgattcaaatatgtggtttcctaataacacaattacttttttttttcttttatcaagaatgatgtattggataccatcatatggcatatttttgataattaaattgccaataatgcagattgatggagatctacataccacaataaagtttattgattcactgaaaatcccttatattgcggcctcgtttggtggctgtgagagcattgtggatcaacctgctattttgtcttactggtactcatttttattccatgttttaagttgtcatgttgatgatttgacactactcggggaatggtcctctacttgaatgtgaccctgtagtgaaagcatcttttgctggtccttttgttaatttttagatttggcttggtggtggttgttgtatgtttaatttaattttcaacatattgtgcaacagggatcttcctcagtcagaaagggccaagtataagatttatgacaacctggttcgcttcagctttggagttgaagattttgaggatttgaaggctgatgtcctgcaagctctggaagctatatagacagttttcctgattcacccatgttttttttcttttaataatcgtgttatatgttccttttcgttatcgcatctggcgattcaattgaattttgatcgtctaatgttctgttggaattgtgtttagatgaatggtttctaatttggatgttgtgaaacttgtgatgaatggttgaaattgaaacctctattgatattcattcccgcgtttcctacaaaaattaaaagaaaaatgaattcttgaggcaggatatataagagaatttctttttgaagcttttgtgatcatgttttctggtaaaaatgggggtgtttgtcctttatattttaccttggtccgttggagctgttttaattttgtttcaaagtagt

SEQ ID NO: 57 - protein, 526 aa, synthetic construct
MAVSSSHMRFTFECRSDPDFSPPPPSFDNLRRRNFRSSAGSGAAFHGISSLILRFPPNFQRQLNIGVAQIVAASWSNNSDNSPAAGAPAPPAATAADAATVPLPVDLAAAEDVVVSAAAAENGTVQLNSSSYSFLKSDASKTIHAAERLGRGIETDGITTPVLNTSAYFFKKTADLIDFKENRQVSYEYGRYGNPTTVVLEEKISILEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDALGGCISGSTKVVSQIRTLHHVLGGTLNPNAAYLFIRGMKTLHLRVQQQNSTGMRMAELLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 58 - DNA, 5123 bp, synthetic construct
  source 1..5123: mol_type = other DNA
agagtacagagagattcgttttggtaggtatatagatagaggagagagaaaccaaacacaaatccttgaaacgacgacggaaatggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcagcaccaattgtagcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccggtacgtaaaattcttgtcttttttttttgcgtcataaattgtgattcatgttttactttccaagattcgtttacttttgaactaaattggatgtgattttctcggataatgtttctttaagtgtactttttcaattcttttatgttttgttagatttttcattattcattacatttggatgtgatttgcttgagaaagagattttctcttgctttttatatgtactgtacataattaatcagtttttcctgcctgtctgacttggtatatttatatttattagtttatatgacgtttgatttaagatttaaagtaggtttcgtggttaatttgtatggattttaattcagttagttttaatatccttggatgttattgttgatggtaatgttgtttacgaagctgattgattttgaattctgaattttggcaatgcagctgaaagactgggtaggggtattgagactgatggaattaccacccctgtgctgaacacttctgcctacttttttaagaaaaccgctgatctcattgatttcaaggttggattgttaaaagatttattcgcctcatgacataatgagtggataaatgtcagttattggtgtggctaaatttttccatcatttggcaggagaatcgtcaagtgagttatgaatacgggcgctatggaaacccaacgactgtggttctggaggagaagataaggtttgtgtgttttgattttgttatttactattaacttgtaatgtgttttaagtgtggtggtcatttgatgaggtttgttgtcattattaaccccagtatattggagggggctgaatcaactgtgataatggcatctggaatgtgtgctagcgtagtcctatttatggcactggttccagctggtggacatcttgtgaccactacggattgttacaggaagactagaatattcattgagacttttcttccaaagatggggatcacggtaaatgttctactatcccattcttttggtggtttaattgtattttgtatttgttgcatggggcagaaactgcttcttatgttgtcacgtttctttagcttaaagagttatttaacttgtttggattttgaaagaatgcttattttcttatgattcaatgctatttggctacttaccaaatcatatcataagatgaatcttaaatgagtttgtatcttacggtacatgaatgaattatcattgatatgaatcacacattcaacatgtgattcattattttattattattttttaaacttttctttctccaattctcatgaaaaattgaatggtcataagtttaaattgttaaatgaaaaggagttgaaggagcacttcggaatttatcctatttgattatttcactactttttatccgtcacacagttattcaatgtatgtactcactactctatttacctatttaattgcttcatttttattcatctaaaataaagccttcacaaactcatatataactaataattttgttttattttttttatttaaaatttgaattttttacattgaacatgatatatttgaagtagtttttcctgttgtaagaatgcttattttctttcctgttgttcaaaattatatttgtgcatacataaattttattcaaattattaaaagaaatctcatggtgaataatattgccgagaatatggtttttggccgagtctaatagcgggcgatgtgtgatccacagagccaacctcgcctagtggaatgaggctattgatgttgttgtagtctttcctagaatataatgattaatagagacacctgatataaatgtcttgctgaaaatttgcttggttttcaactggaaatttgaaatggatatcttaaatgtgaatcttcttgtcagttgattgagtaaggacataaggtggacttttgacattttcaattttttatttctggtaggtttttactgttgtcctgggtgcttgcaatatatgtaacatattttccctttgatgaacagaccactgtaattgatccagcagatgttggagccttggaatctgcattggagcagcacaatgttagtgttggagaaagtagttggttttcttttatggatttctctgatttatttgtagttaatagatggtcttgagttaacatggtatgatacaatcattcaggtgtctctattcttcactgagtcgcctaccaatccattcctgagatgtgttgacattaagctggtttcagagctttgccacaagaaggggactttgctctgtattgatggtacatttgcaacaccattgaaccagaaggcccttgcccttggcgctgatctgattctgcactccttaacaaaatacatgggtggacatcatgatgtaagttggcatgttcaagatttagtttgatggaaaaacaacacaaatatgttacatttcagttgttcagagttaagcgtcaaggagcatttttcctctttcaggcccttggtggttgcataagtggttcaactaaggtggtttcgcaaattcggactttgcaccatgttttgggtggtacacttaacccggtaagtgctttctatagagctgagcctccatggaaggatatcgtaattggttattgtggataacatagtgccaatttttccgcagaatgctgcatacctattcatcagaggcatgaaaacgctgcatctccgtgtacagcagcagaattcaacaggaatgaggatggccgaacttttagaggcacatcccaaggtatcatggcattttgacatgcacactgtcatgcctcgtctccatgctatttaatataatttattattttgttaattgaaacttttaaagttgtcaaatgagattgtgtataattcaattgaattcaaatatataaaaaatgaaatgaatggttaacacatgacttgtttcttcatcgacagcttatttttgttgctgatctgtcttttaaactttttttcccctatgtatatttgtgtataatgatggttgaattggggttctacgcaaaaatgaagtgtatggttaaatgtcatatcttgttcatcaacagcatacttatgttggaatattgtgcactgctccctatgtactctaaaatgtatgtggttgtagcatgatggataatgtttactaagctgtgccaagagagcagatgctggtttcttttgaattttttttgttgtgatgctcttaaatgggagtcccttcatctgctattatcttgtatattaatgataataagtaattaagtattattacttttttgatagtagcttttaggatggaagcctaagtatttttgtggcataaaatttctcttgaaacaaaactgtattcttctctaggttcttcctttttttttccactttttgaattgtcaaatcagttgtttgtatcttttctcttctttttgatgtcattaacaaaggcatattcttataccattttatcattttacagattcagtatgtaaacattttcatgattgagtcctgtatctgatgtggctttaattacatactattattggcaggtgaagcgggtctactatccaggcttgccgagtcatcctgaacatgagcttgccaagaggcagatgactggtttcggtggtgttgtcagttttgaggtgggaatttgctaagttgcgattcaaatatgtggtttcctaataacacaattacttttttttttcttttatcaagaatgatgtattggataccatcatatggcatatttttgataattaaattgccaataatgcagattgatggagatctacataccacaataaagtttattgattcactgaaaatcccttatattgcggcctcgtttggtggctgtgagagcattgtggatcaacctgctattttgtcttactggtactcatttttattccatgttttaagttgtcatgttgatgatttgacactactcggggaatggtcctctacttgaatgtgaccctgtagtgaaagcatcttttgctggtccttttgttaatttttagatttggcttggtggtggttgttgtatgtttaatttaattttcaacatattgtgcaacagggatcttcctcagtcagaaagggccaagtataagatttatgacaacctggttcgcttcagctttggagttgaagattttgaggatttgaaggctgatgtcctgcaagctctggaagctatatagacagttttcctgattcacccatgttttttttcttttaataatcgtgttatatgttccttttcgttatcgcatctggcgattcaattgaattttgatcgtctaatgttctgttggaattgtgtttagatgaatggtttctaatttggatgttgtgaaacttgtgatgaatggttgaaattgaaacctctattgatattcattcccgcgtttcctacaaaaattaaaagaaaaatgaattcttgaggcaggatatataagagaatttctttttgaagcttttgtgatcatgttttctggtaaaaatgggggtgtttgtcctttatattttaccttggtccgttggagctgttttaattttgtttcaaagtagt

SEQ ID NO: 59 - protein, 531 aa, synthetic construct
MAVSSSHMRFTFECRSDPDFSPPPPSFDNLRRRNFRSSAGSGAAFHGISSLILRFPPNFQRQLSTNCSNIGVAQIVAASWSNNSDNSPAAGAPAPPAATAADAATVPLPVDLAAAEDVVVSAAAAENGTVQLNSSSYSFLKSDASKTIHAAERLGRGIETDGITTPVLNTSAYFFKKTADLIDFKENRQVSYEYGRYGNPTTVVLEEKISILEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDALGGCISGSTKVVSQIRTLHHVLGGTLNPNAAYLFIRGMKTLHLRVQQQNSTGMRMAELLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 60 - DNA, 5137 bp, synthetic construct
  source 1..5137: mol_type = other DNA
agagtacagagagattcgttttggtaggtatatagatagaggagagagaaaccaaacacaaatccttgaaacgacgacggaaatggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcagcaccaaggtcgcgccgcaattggtagcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccggtacgtaaaattcttgtcttttttttttgcgtcataaattgtgattcatgttttactttccaagattcgtttacttttgaactaaattggatgtgattttctcggataatgtttctttaagtgtactttttcaattcttttatgttttgttagatttttcattattcattacatttggatgtgatttgcttgagaaagagattttctcttgctttttatatgtactgtacataattaatcagtttttcctgcctgtctgacttggtatatttatatttattagtttatatgacgtttgatttaagatttaaagtaggtttcgtggttaatttgtatggattttaattcagttagttttaatatccttggatgttattgttgatggtaatgttgtttacgaagctgattgattttgaattctgaattttggcaatgcagctgaaagactgggtaggggtattgagactgatggaattaccacccctgtgctgaacacttctgcctacttttttaagaaaaccgctgatctcattgatttcaaggttggattgttaaaagatttattcgcctcatgacataatgagtggataaatgtcagttattggtgtggctaaatttttccatcatttggcaggagaatcgtcaagtgagttatgaatacgggcgctatggaaacccaacgactgtggttctggaggagaagataaggtttgtgtgttttgattttgttatttactattaacttgtaatgtgttttaagtgtggtggtcatttgatgaggtttgttgtcattattaaccccagtatattggagggggctgaatcaactgtgataatggcatctggaatgtgtgctagcgtagtcctatttatggcactggttccagctggtggacatcttgtgaccactacggattgttacaggaagactagaatattcattgagacttttcttccaaagatggggatcacggtaaatgttctactatcccattcttttggtggtttaattgtattttgtatttgttgcatggggcagaaactgcttcttatgttgtcacgtttctttagcttaaagagttatttaacttgtttggattttgaaagaatgcttattttcttatgattcaatgctatttggctacttaccaaatcatatcataagatgaatcttaaatgagtttgtatcttacggtacatgaatgaattatcattgatatgaatcacacattcaacatgtgattcattattttattattattttttaaacttttctttctccaattctcatgaaaaattgaatggtcataagtttaaattgttaaatgaaaaggagttgaaggagcacttcggaatttatcctatttgattatttcactactttttatccgtcacacagttattcaatgtatgtactcactactctatttacctatttaattgcttcatttttattcatctaaaataaagccttcacaaactcatatataactaataattttgttttattttttttatttaaaatttgaattttttacattgaacatgatatatttgaagtagtttttcctgttgtaagaatgcttattttctttcctgttgttcaaaattatatttgtgcatacataaattttattcaaattattaaaagaaatctcatggtgaataatattgccgagaatatggtttttggccgagtctaatagcgggcgatgtgtgatccacagagccaacctcgcctagtggaatgaggctattgatgttgttgtagtctttcctagaatataatgattaatagagacacctgatataaatgtcttgctgaaaatttgcttggttttcaactggaaatttgaaatggatatcttaaatgtgaatcttcttgtcagttgattgagtaaggacataaggtggacttttgacattttcaattttttatttctggtaggtttttactgttgtcctgggtgcttgcaatatatgtaacatattttccctttgatgaacagaccactgtaattgatccagcagatgttggagccttggaatctgcattggagcagcacaatgttagtgttggagaaagtagttggttttcttttatggatttctctgatttatttgtagttaatagatggtcttgagttaacatggtatgatacaatcattcaggtgtctctattcttcactgagtcgcctaccaatccattcctgagatgtgttgacattaagctggtttcagagctttgccacaagaaggggactttgctctgtattgatggtacatttgcaacaccattgaaccagaaggcccttgcccttggcgctgatctgattctgcactccttaacaaaatacatgggtggacatcatgatgtaagttggcatgttcaagatttagtttgatggaaaaacaacacaaatatgttacatttcagttgttcagagttaagcgtcaaggagcatttttcctctttcaggcccttggtggttgcataagtggttcaactaaggtggtttcgcaaattcggactttgcaccatgttttgggtggtacacttaacccggtaagtgctttctatagagctgagcctccatggaaggatatcgtaattggttattgtggataacatagtgccaatttttccgcagaatgctgcatacctattcatcagaggcatgaaaacgctgcatctccgtgtacagcagcagaattcaacaggaatgaggatggccgaacttttagaggcacatcccaaggtatcatggcattttgacatgcacactgtcatgcctcgtctccatgctatttaatataatttattattttgttaattgaaacttttaaagttgtcaaatgagattgtgtataattcaattgaattcaaatatataaaaaatgaaatgaatggttaacacatgacttgtttcttcatcgacagcttatttttgttgctgatctgtcttttaaactttttttcccctatgtatatttgtgtataatgatggttgaattggggttctacgcaaaaatgaagtgtatggttaaatgtcatatcttgttcatcaacagcatacttatgttggaatattgtgcactgctccctatgtactctaaaatgtatgtggttgtagcatgatggataatgtttactaagctgtgccaagagagcagatgctggtttcttttgaattttttttgttgtgatgctcttaaatgggagtcccttcatctgctattatcttgtatattaatgataataagtaattaagtattattacttttttgatagtagcttttaggatggaagcctaagtatttttgtggcataaaatttctcttgaaacaaaactgtattcttctctaggttcttcctttttttttccactttttgaattgtcaaatcagttgtttgtatcttttctcttctttttgatgtcattaacaaaggcatattcttataccattttatcattttacagattcagtatgtaaacattttcatgattgagtcctgtatctgatgtggctttaattacatactattattggcaggtgaagcgggtctactatccaggcttgccgagtcatcctgaacatgagcttgccaagaggcagatgactggtttcggtggtgttgtcagttttgaggtgggaatttgctaagttgcgattcaaatatgtggtttcctaataacacaattacttttttttttcttttatcaagaatgatgtattggataccatcatatggcatatttttgataattaaattgccaataatgcagattgatggagatctacataccacaataaagtttattgattcactgaaaatcccttatattgcggcctcgtttggtggctgtgagagcattgtggatcaacctgctattttgtcttactggtactcatttttattccatgttttaagttgtcatgttgatgatttgacactactcggggaatggtcctctacttgaatgtgaccctgtagtgaaagcatcttttgctggtccttttgttaatttttagatttggcttggtggtggttgttgtatgtttaatttaattttcaacatattgtgcaacagggatcttcctcagtcagaaagggccaagtataagatttatgacaacctggttcgcttcagctttggagttgaagattttgaggatttgaaggctgatgtcctgcaagctctggaagctatatagacagttttcctgattcacccatgttttttttcttttaataatcgtgttatatgttccttttcgttatcgcatctggcgattcaattgaattttgatcgtctaatgttctgttggaattgtgtttagatgaatggtttctaatttggatgttgtgaaacttgtgatgaatggttgaaattgaaacctctattgatattcattcccgcgtttcctacaaaaattaaaagaaaaatgaattcttgaggcaggatatataagagaatttctttttgaagcttttgtgatcatgttttctggtaaaaatgggggtgtttgtcctttatattttaccttggtccgttggagctgttttaattttgtttcaaagtagt

SEQ ID NO: 61 - DNA, 5136 bp, synthetic construct
  source 1..5136: mol_type = other DNA
agagtacagagagattcgttttggtaggtatatagatagaggagagagaaaccaaacacaaatccttgaaacgacgacggaaatggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcagcaccaaggtcgcgccgcaattgtagcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccggtacgtaaaattcttgtcttttttttttgcgtcataaattgtgattcatgttttactttccaagattcgtttacttttgaactaaattggatgtgattttctcggataatgtttctttaagtgtactttttcaattcttttatgttttgttagatttttcattattcattacatttggatgtgatttgcttgagaaagagattttctcttgctttttatatgtactgtacataattaatcagtttttcctgcctgtctgacttggtatatttatatttattagtttatatgacgtttgatttaagatttaaagtaggtttcgtggttaatttgtatggattttaattcagttagttttaatatccttggatgttattgttgatggtaatgttgtttacgaagctgattgattttgaattctgaattttggcaatgcagctgaaagactgggtaggggtattgagactgatggaattaccacccctgtgctgaacacttctgcctacttttttaagaaaaccgctgatctcattgatttcaaggttggattgttaaaagatttattcgcctcatgacataatgagtggataaatgtcagttattggtgtggctaaatttttccatcatttggcaggagaatcgtcaagtgagttatgaatacgggcgctatggaaacccaacgactgtggttctggaggagaagataaggtttgtgtgttttgattttgttatttactattaacttgtaatgtgttttaagtgtggtggtcatttgatgaggtttgttgtcattattaaccccagtatattggagggggctgaatcaactgtgataatggcatctggaatgtgtgctagcgtagtcctatttatggcactggttccagctggtggacatcttgtgaccactacggattgttacaggaagactagaatattcattgagacttttcttccaaagatggggatcacggtaaatgttctactatcccattcttttggtggtttaattgtattttgtatttgttgcatggggcagaaactgcttcttatgttgtcacgtttctttagcttaaagagttatttaacttgtttggattttgaaagaatgcttattttcttatgattcaatgctatttggctacttaccaaatcatatcataagatgaatcttaaatgagtttgtatcttacggtacatgaatgaattatcattgatatgaatcacacattcaacatgtgattcattattttattattattttttaaacttttctttctccaattctcatgaaaaattgaatggtcataagtttaaattgttaaatgaaaaggagttgaaggagcacttcggaatttatcctatttgattatttcactactttttatccgtcacacagttattcaatgtatgtactcactactctatttacctatttaattgcttcatttttattcatctaaaataaagccttcacaaactcatatataactaataattttgttttattttttttatttaaaatttgaattttttacattgaacatgatatatttgaagtagtttttcctgttgtaagaatgcttattttctttcctgttgttcaaaattatatttgtgcatacataaattttattcaaattattaaaagaaatctcatggtgaataatattgccgagaatatggtttttggccgagtctaatagcgggcgatgtgtgatccacagagccaacctcgcctagtggaatgaggctattgatgttgttgtagtctttcctagaatataatgattaatagagacacctgatataaatgtcttgctgaaaatttgcttggttttcaactggaaatttgaaatggatatcttaaatgtgaatcttcttgtcagttgattgagtaaggacataaggtggacttttgacattttcaattttttatttctggtaggtttttactgttgtcctgggtgcttgcaatatatgtaacatattttccctttgatgaacagaccactgtaattgatccagcagatgttggagccttggaatctgcattggagcagcacaatgttagtgttggagaaagtagttggttttcttttatggatttctctgatttatttgtagttaatagatggtcttgagttaacatggtatgatacaatcattcaggtgtctctattcttcactgagtcgcctaccaatccattcctgagatgtgttgacattaagctggtttcagagctttgccacaagaaggggactttgctctgtattgatggtacatttgcaacaccattgaaccagaaggcccttgcccttggcgctgatctgattctgcactccttaacaaaatacatgggtggacatcatgatgtaagttggcatgttcaagatttagtttgatggaaaaacaacacaaatatgttacatttcagttgttcagagttaagcgtcaaggagcatttttcctctttcaggcccttggtggttgcataagtggttcaactaaggtggtttcgcaaattcggactttgcaccatgttttgggtggtacacttaacccggtaagtgctttctatagagctgagcctccatggaaggatatcgtaattggttattgtggataacatagtgccaatttttccgcagaatgctgcatacctattcatcagaggcatgaaaacgctgcatctccgtgtacagcagcagaattcaacaggaatgaggatggccgaacttttagaggcacatcccaaggtatcatggcattttgacatgcacactgtcatgcctcgtctccatgctatttaatataatttattattttgttaattgaaacttttaaagttgtcaaatgagattgtgtataattcaattgaattcaaatatataaaaaatgaaatgaatggttaacacatgacttgtttcttcatcgacagcttatttttgttgctgatctgtcttttaaactttttttcccctatgtatatttgtgtataatgatggttgaattggggttctacgcaaaaatgaagtgtatggttaaatgtcatatcttgttcatcaacagcatacttatgttggaatattgtgcactgctccctatgtactctaaaatgtatgtggttgtagcatgatggataatgtttactaagctgtgccaagagagcagatgctggtttcttttgaattttttttgttgtgatgctcttaaatgggagtcccttcatctgctattatcttgtatattaatgataataagtaattaagtattattacttttttgatagtagcttttaggatggaagcctaagtatttttgtggcataaaatttctcttgaaacaaaactgtattcttctctaggttcttcctttttttttccactttttgaattgtcaaatcagttgtttgtatcttttctcttctttttgatgtcattaacaaaggcatattcttataccattttatcattttacagattcagtatgtaaacattttcatgattgagtcctgtatctgatgtggctttaattacatactattattggcaggtgaagcgggtctactatccaggcttgccgagtcatcctgaacatgagcttgccaagaggcagatgactggtttcggtggtgttgtcagttttgaggtgggaatttgctaagttgcgattcaaatatgtggtttcctaataacacaattacttttttttttcttttatcaagaatgatgtattggataccatcatatggcatatttttgataattaaattgccaataatgcagattgatggagatctacataccacaataaagtttattgattcactgaaaatcccttatattgcggcctcgtttggtggctgtgagagcattgtggatcaacctgctattttgtcttactggtactcatttttattccatgttttaagttgtcatgttgatgatttgacactactcggggaatggtcctctacttgaatgtgaccctgtagtgaaagcatcttttgctggtccttttgttaatttttagatttggcttggtggtggttgttgtatgtttaatttaattttcaacatattgtgcaacagggatcttcctcagtcagaaagggccaagtataagatttatgacaacctggttcgcttcagctttggagttgaagattttgaggatttgaaggctgatgtcctgcaagctctggaagctatatagacagttttcctgattcacccatgttttttttcttttaataatcgtgttatatgttccttttcgttatcgcatctggcgattcaattgaattttgatcgtctaatgttctgttggaattgtgtttagatgaatggtttctaatttggatgttgtgaaacttgtgatgaatggttgaaattgaaacctctattgatattcattcccgcgtttcctacaaaaattaaaagaaaaatgaattcttgaggcaggatatataagagaatttctttttgaagcttttgtgatcatgttttctggtaaaaatgggggtgtttgtcctttatattttaccttggtccgttggagctgttttaattttgtttcaaagtagt

SEQ ID NO: 62 - DNA, 5221 bp, synthetic construct
  source 1..5221: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggagtgtttgttgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 63 - DNA, 5219 bp, synthetic construct
  source 1..5219: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggagtttgttgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 64 - DNA, 5214 bp, synthetic construct
  source 1..5214: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggattgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 65 - DNA, 5215 bp, synthetic construct
  source 1..5215: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaattgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 66 - DNA, 5220 bp, synthetic construct
  source 1..5220: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggatgtttgttgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 67 - DNA, 5220 bp, synthetic construct
  source 1..5220: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagctatggtgtttgttgtccctcccggtcatcccttcgtcaccatagcttccaataaagagaatctcctcatgatttgcttcgaggttaacgctcgagacaacaagaagtttacatttgcaggtaggcacacacacataaacccttagttatgccatgcatacattttttttatgcatgcatgttcatttggtttttaattagtttattgcaaagttgaagctagttattatatatattgtgtgtaacagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 68 - DNA, 4926 bp, synthetic construct
  source 1..4926: mol_type = other DNA
atgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagccatggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaagggtacgtaaatttaaattaagcaaaatatatattctttttagtttaagatatttgcatatgagttttgacttctgagtttaatgttgatgcatgcatttactaaccatttaaaataattttatacttttattcaatcataaaatattatttttataacttttaaaataattattatatttatataataataagttataattagacgtcatataaaattattttacactatattttttcttttatatatatatatatataatattaatttattgatgctttttttattaatttgttggtgattcacacatctactgttgatgctgtacaattttgtgaaatttccttacttcatttgttgtatttctatttatttactagggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggtaattgcttctaattatatatacatatggattagttaagtttttaataacatgcaaaagtctagagaactagaccaaacaaagttttgatcattaattaaaaatcaaatttcaaaatctttagaaactaaatcaatactcacaattgagttcacatgttaactaattaaagcaagtagaagatggtatatacgatatagttgaatattatattttatactgatcgaaggaaattgtggtgtaggaatttttcgggcctggaggacgagacccagaatcggtcctctcagcattcagctggaatgtgctgcaagctgcgctccaagtatatagacccttaatttctagctatcatgcatcaatattatattatagcataaagtaacattaaatgctactaatttggcttgtcaatttcattaacttggtttcttgtattttttaccaaaatggaaaataattacagaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccaggtatattatcaacacgcttatcttatctttatttatttatctcattaactgatgagtaagttactagtttttgaactgttatcaactctaatgtagactacgtttattaatttcaaacaataattctcttaattggttgtcatttttttatcagtttggttaatatcttggttgccatctatagtaaatagaacttaaattcttggtaatctttcaagaaaaaacagtacatgtatatatagtgtaaagatcattgacattatcaaccaatttgatattgttcaatggtagtctttttggctttatcataattaaatttttcttaacatgaaaacaagccttgtgactgataacattatattatttccataagatattgtcatggatggtagctagttttttcttacttttataataattaacttattctttaagatgataacgtgatctgggactgatcttgacagtttaaaatttactgcatagatttgaactcaattcttaataatgatttgcatgttctcctataatttaatagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagggctacaaatcaagaaaaggagacaacaaacggagtattcgaccgaaaggagagtttctttttcccttttgagttgccgagagaggagcgtggtcgtcgcgctgatgcgtgagaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaagtctaaattccttaaatcatgaatacttacataagaaaagaaaaaagaaaaaaataaaataactatcattataagt

SEQ ID NO: 69 - DNA, 4134 bp, synthetic construct
  source 1..4134: mol_type = other DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggagtgtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 70 - DNA, 4135 bp, synthetic construct
  source 1..4135: mol_type = other DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaagtgtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 71 - DNA, 4132 bp, synthetic construct
  source 1..4132: mol_type = other DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggagtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 72 - DNA, 4136 bp, synthetic construct
  source 1..4136: mol_type = other DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggatggtgtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 73 - DNA, 4123 bp, synthetic construct
  source 1..4123: mol_type = other DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 74 - DNA, 4136 bp, synthetic construct; identical to SEQ ID NO: 72
  source 1..4136: mol_type = other DNA

SEQ ID NO: 75 - DNA, 4133 bp, synthetic construct
  source 1..4133: mol_type = other DNA
gtgtagattgacatgagtatacgcacgcacacccagctctacttagcagcaattacccgttttacttgctacttaagagacacgtacattaacacttgtccttgtgcatgcaattgccaccacattcctcactccacccttttctttatatataaacaaacacaatggatcatctcaaaccaagagtgagtttgttgttcctcactgacctcaccatggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggaggaagacgaaaatccctacgtttttgaagaagataaggatttttcgaccagagtcgaaacagaaggtggcagcattcgggttctcaagaagttcactgagaaatccaagcttcttcaaggcattgagaatttccgtttggccatcttagaagctagagcacacacgttcgtgtccccacgccactttgattccgaggttgtcttgttcaacattaagggtgcgtatatttgaattaaacaaaaaatacatttttttttgttagtttcagttatgtacccgagtttaatagtgattacactctaattttatatcattattcaatcatacattaatatttatatgatttttagaataattattttttttaatgaaactttatgataggtttttgtcttagatgacaatatatcaattaatttattgatgccttttttattattaatttgttggttattcacacatctactgctcatgctgtacaattttgtgaaatttcctcctttcattattagttgtattactatttatttactagggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggtaattatatatacataagtgctagttatgcttttagtttttaaatttttatccgttaagatttttttctgaacaaaagtttgactttgacttcagtctacaattttttaaaaatttgatttttaatttatgaagagaagtttgaatccttttattttcatttgtttaacagatttttttagatgaaaatcgaatttaaaaaaaaaagtttaagactgcttaaacttttgttcaagaacgaacaatcttaataggaaaaaattaaaaaaactagaccaatcaatttttttttactaagaaatgaatttaaaaaatctttaaaaactaaagacttagttaatcttgtacacacatacatgtacatcaactttacaatttacaaacttaattcagtacatttatgaatatacatgtatgtatatgtgtgtgtgtgtatatatatatataaactttacaatttacaaacttaattcagattgactaaatcaatactcacgattgagttcacacatgttaactaattaaacaaagtagaagatagtatatagttgaatattatatttaaatttatactgatggaagaaaattgtggtgtaggaatttttcgggcctggaggacgagacccagaatcggtcctctcagcattcagctggaatgtgctgcaagctgcgctccaagtatatagacccttaatttcttgctatcatgcatcattaacattaaatgctactaattaatttggctttgtcaatttgatttctttttattttgggaaattaattacagaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccaggtatatatattaccaacatgcttatcttaattttttttacttgatagtcataaggaaaagattgtgagtttgatttcatccgctaataacatactaataatttaataactaatatttactgattaattaaaaaaatgtttattaacttcaaagaaaatttatcttggttgtcatttttttatcagttcagtttggtttatatattggttgtcatctagtacataaaacttaaattctttgtaatttttcaagagaaaacagtacatgtatagtgtaattaagtagcatttacattatcaaccaataagatatttcttattgtatggatggtatagtctttttgactttataataattaactttttcataacatgataacattatttgtgactgatagatagaataattacaataagatatcgttttagatggttgtctttttgaataataattaacttattcttaacatcataacatgatctgtgactttaaaatttactgcatagatttgaactcaattcttaattgataactaataataatgatgttacatgttatcctaattaaatagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctgggtgtttgttgtccctcctggtcatcccttcgtcactatagcttccaataaagagaatctcctcataatttgcttcgaggttaacgttcgagacaacaagaagtttacgtttgcaggtaggagtacacataaatccttaataatgccatgcatacatttttttttaacgtgtagggatcaaagaataattcacgtatctttttaaccaattaaattaaaaaagatatattataacaattgaactattgtgaagcataaaagaacaaaattcttatttagttttaaagcaataaaaatatgcaaataattgaaaaggtgaaagaaagagagaaaaatatataatgatataaaattctaatatatatctaaataaaaatatacagataaatgaaaaggtgaaagaaagagagaaaaatatatcaatctaaattaatttatatctatttatatatgaattaatttataacttaaacaaattaagaggtctgaatcaataaaaattcaaaattgagaatattgattaaaaaaaacaaatgttaattttacgaactgaatttaaccctcttatttgtcatgtgtcatgcatgcatgttgatttggttcataacgagttgaagctaattatatatattgtgtgtggaacagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcgtgagaagcagtgtggaggtggctgataacggggaatgtatttagctttgagagtctttaaattttctgtatttgttgtaatgttagtagttccttaaattggccagatggagtttatgtgtttgtaaatgcagggatgctaacggaataaaatggccacttgtattgctatcccttgtgctagcataatagaaagggttgaatcgagctaatccgagtttcattcagtttaactcgagttttttttaaaatgttagtagttcctttaacttctcaaggacgcttctattaatcttcaaatatgaaaaaagtaaaagatgattaacgtagtt

SEQ ID NO: 76 - DNA, 2240 bp, synthetic construct
  source 1..2240: mol_type = other DNA
atcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaagaacttccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatat

SEQ ID NO: 77 - DNA, 2781 bp, synthetic construct
  source 1..2781: mol_type = other DNA
atcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagtcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgaggtaagcagaaaaactgaacatgacaattgagttctattcactttcttcttagttagagaaacctattcttgagactttaaataatgatttacttttttctttgttcacaaatatagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagagtccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatat

SEQ ID NO: 78 - DNA, 2788 bp, synthetic construct
  source 1..2788: mol_type = other DNA
atcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagttcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgaggtaagcagaaaaactgaacatgacaattgagttctattcactttcttcttagttagagaaacctattcttgagactttaaataatgatttacttttttctttgttcacaaatatagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgcccaagaacaaccagaggaacttccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatat

SEQ ID NO: 79 - DNA, 2781 bp, synthetic construct
  source 1..2781: mol_type = other DNA
atcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatcaaggaaaaccatttcttctgaagataaacctttcaacttgagaagccgcgaccccatctattccaacaagcttggcaagttgtttgagattaccccagagaaaaaccctcagcttcgggacttggatgtcttcctcagtgttgtggatatgaacgaggtaagcagaaaaactgaacatgacaattgagttctattcactttcttcttagttagagaaacctattcttgagactttaaataatgatttacttttttctttgttcacaaatatagggagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagaggaacttccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatat

SEQ ID NO: 80 - DNA, 2787 bp, synthetic construct
  source 1..2787: mol_type = other DNA
atcaagaaccagttcaatatcctagtacaccgtattaaagaatttaagatatactatgatgagagcgcggttcccattactgttgctgggagttgttttcctggcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaagaatgcgaagaaggtcaaattccacgaccacgaccacaacacccggagagggaacgtcagcaacacggtgagaaggaggaagacgaaggtgagcagccacgtccattcccattcccacgcccacgccaacctcatcaagaggaagagcacgagcagaaggaggaacacgaatggcatcgcaaggaggaaaaacacggaggaaagggaagtgaagaggaacaagatgaacgtgaacacccacgcccacaccaacctcatcaaaaggaagaggaaaagcacgaatggcaacacaagcaggaaaagcaccaaggaaaggaaagtgaagaagaagaagaagaccaagacgaggatgaggagcaagacaaagagagccaagaaagtgaaggttctgagtctcaaagagaaccacgaagacataagaataagaacccttttcacttcaactctaaaaggttccaaactctcttcaaaaaccaatatggccacgttcgcgtcctccagaggttcaacaaacgctcccaacagcttcagaatctccgagactaccgcattttggagttcaactccaaacccaacacccttcttctcccccaccatgctgacgctgattacctcatcgttatccttaacggttagtattcctttcctctcaaaataaaataaatgtcattattttatgcggattaagaaaaattattaaataaataaaaacaaaaccaacaattttataaaaactaatcttactatgacacttatttaattaattaataaacatcatcacatgcttttacttctcataaaattatgttattgaatttgttttgttaattacagggactgccattcttaccttggtgaacaacgacgaccgagactcttacaaccttcaatctggcgatgccctaagagtccctgcaggaaccacatactatgtggttaaccctgacaacgacgagaatctcagaatgataacactcgccatacccgttaacaaacccggtagatttgaggtactacttattctttataactaattaattaattattattctaatttgtttccaaatcttaagatcaatctttttcttttctctgcagagtttcttcctatctagcactcaagctcaacagtcctacttgcaagggttcagcaagaatattctagaggcctcatacgacgtaagcaaccaacacaccatctaatacgctagcaaattcaatattatcattatccttatatttgtttccgcgcttgattttatagaccaaattcgaggagataaacaaggttctgtttggtagagaggaggggcagcaacaaggggaggagaggctgcaagagagtgtgattgtggaaatctcaaagaaacaaattcgggaactgagcaaacatgccaaatctagttcctctggttgttctcggcattgataccaaaagcaaagaaattcagatctgaggtagcgttgaccacaactggataacctgctgggattacaaatatatcttgttcagacaattcagctctatatttccgcacttccaaaggttgctcttcctgttgctgcctctgttgttgttctttaatgccaacaagttcaatgtttgcttctccttcattaatcactagtaccactatggcctttgaattgaagtgtggcagaaaaagagctccctatatttgtgaacaaagaaaaaagtaaatcattatttaaagtctcaagaataggtttctctaactaagaagaaagtgaatagaactcaattgtcatgttcagtttttctgcttacctcgttcatatccacaacactgaggaagacatccaagtcccgaagctgagggtttttctctggggtaatctcaaacaacttgccaagcttgttggaatagatggggtcgcggcttctcaagttgaaaggtttatcttcagaagaaatggttttccttgaacttccttgcaggtacatattttgtatatcacgatcaaatagaacatgctgttgaagtgttgttactttttttgttttattaattacatgtgaaacatagctgactgagctatttctaatcctttggtgattgaaaatttgaaggttcgaaagacaatgtgataagccagatacctagtcaagtgcaggagcttgcgttccctgggtctgcaaaagatattgagaacctaataaagagccaaagtgagtcctactttgtggatgctcagcctcagcagaaagaggaggggaacaagggaagaaagggtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatat

SEQ ID NO: 81 - DNA, 2669 bp, synthetic construct
  source 1..2669: mol_type = other DNA
aaccaccccaattttgttcaacaattcaacacacgtcactctgcatgcccatcctgtggccaaattttcatgcaagttaaccaaagtcgctttttctcttctgtaactatatatatatatatatatatatatatatatatagctctaatctcacaccgtgttttcatcatcacacaagagcgagcgcaacattattcgagtacgtagcttaacaatattttaagcaattgctgctacaatgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcggttagtacacttaactaattactactactaattaaacatcgatcatcactttgcttctgctcttagtactagttaatggtaatcaattttactaattacagggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggtactgcatgcctttttcattatgacagattagttaattaattcttctaattaatttatttccaaatgtgaagtgaatatcaatctttgtccttttctctttctctaggatttcttcctatctagcactcaagaccagcaatcctacttgcaaggcttcagcgagaatattctggaggcttccttcaatgtaagcaagcaacacatgcaccatctaatttgcatttccgtttagtggtttgtctaaatgtcatactaattaaatattatcattatatttgcttgtgtgcgcaattggttttatagaccaaattcgaggagataaacagggttttgtttggtggagaggggaggcggcaccagcaagagggagtgattttggaactatcaaaggaacagattcgggaactgagcaagcgtgccaaattcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgaggtaagtgcaaaaaccatgtagatctagacaaagtgaactgcactattgagttctattcactctcttcttaattagttagttatactaattattgagactaaacaaatacagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagagtccttgcaggtactatacatatataatattgattattgaataccctgttgttactttttgttgttttatggcatgtggaagcatgctgattgggttatttaattatttccaattcttggtgcgtgtgattgaaaatttgaaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttgaatatgtatgcttaggagactaggagttcacagcgagcgaggaaagtatagggatatgtaactagaataacttgagatccacctttctactactagtaaataaaataaataaatcgag

SEQ ID NO: 82 - DNA, 2676 bp, synthetic construct
  source 1..2676: mol_type = other DNA
aaccaccccaattttgttcaacaattcaacacacgtcactctgcatgcccatcctgtggccaaattttcatgcaagttaaccaaagtcgctttttctcttctgtaactatatatatatatatatatatatatatatatatagctctaatctcacaccgtgttttcatcatcacacaagagcgagcgcaacattattcgagtacgtagcttaacaatattttaagcaattgctgctacaatgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcggttagtacacttaactaattactactactaattaaacatcgatcatcactttgcttctgctcttagtactagttaatggtaatcaattttactaattacagggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggtactgcatgcctttttcattatgacagattagttaattaattcttctaattaatttatttccaaatgtgaagtgaatatcaatctttgtccttttctctttctctaggatttcttcctatctagcactcaagaccagcaatcctacttgcaaggcttcagcgagaatattctggaggcttccttcaatgtaagcaagcaacacatgcaccatctaatttgcatttccgtttagtggtttgtctaaatgtcatactaattaaatattatcattatatttgcttgtgtgcgcaattggttttatagaccaaattcgaggagataaacagggttttgtttggtggagaggggaggcggcaccagcaagagggagtgattttggaactatcaaaggaacagattcgggaactgagcaagcgtgccaaatctagtttcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgaggtaagtgcaaaaaccatgtagatctagacaaagtgaactgcactattgagttctattcactctcttcttaattagttagttatactaattattgagactaaacaaatacagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagagttccttgcaggtactatacatatataatattgattattgaataccctgttgttactttttgttgttttatggcatgtggaagcatgctgattgggttatttaattatttccaattcttggtgcgtgtgattgaaaatttgaaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttgaatatgtatgcttaggagactaggagttcacagcgagcgaggaaagtatagggatatgtaactagaataacttgagatccacctttctactactagtaaataaaataaataaatcgag

SEQ ID NO: 83 - DNA, 2145 bp, synthetic construct
  source 1..2145: mol_type = other DNA
aaccaccccaattttgttcaacaattcaacacacgtcactctgcatgcccatcctgtggccaaattttcatgcaagttaaccaaagtcgctttttctcttctgtaactatatatatatatatatatatatatatatatatagctctaatctcacaccgtgttttcatcatcacacaagagcgagcgcaacattattcgagtacgtagcttaacaatattttaagcaattgctgctacaatgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcggttagtacacttaactaattactactactaattaaacatcgatcatcactttgcttctgctcttagtactagttaatggtaatcaattttactaattacagggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggtactgcatgcctttttcattatgacagattagttaattaattcttctaattaatttatttccaaatgtgaagtgaatatcaatctttgtccttttctctttctctaggatttcttcctatctagcactcaagaccagcaatcctacttgcaaggcttcagcgagaatattctggaggcttccttcaatgtaagcaagcaacacatgcaccatctaatttgcatttccgtttagtggtttgtctaaatgtcatactaattaaatattatcattatatttgcttgtgtgcgcaattggttttatagaccaaattcgaggagataaacagggttttgtttggtggagaggggaggcggcaccagcaagagggagtgattttggaactatcaaaggaacagattcgggaactgagcaagcgtgccaaatctagtggtactatacatatataatattgattattgaataccctgttgttactttttgttgttttatggcatgtggaagcatgctgattgggttatttaattatttccaattcttggtgcgtgtgattgaaaatttgaaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttgaatatgtatgcttaggagactaggagttcacagcgagcgaggaaagtatagggatatgtaactagaataacttgagatccacctttctactactagtaaataaaataaataaatcgag

SEQ ID NO: 84 - DNA, 2677 bp, synthetic construct
  source 1..2677: mol_type = other DNA
aaccaccccaattttgttcaacaattcaacacacgtcactctgcatgcccatcctgtggccaaattttcatgcaagttaaccaaagtcgctttttctcttctgtaactatatatatatatatatatatatatatatatatagctctaatctcacaccgtgttttcatcatcacacaagagcgagcgcaacattattcgagtacgtagcttaacaatattttaagcaattgctgctacaatgatagcgcggtttcctttgttgccgttgctgttgctgggaattgtttttctagcatcagtttctgtctcattacaggtgaagccgccgagtcaccacgagtgcctccagagttgcgagagtgaaacggatccatacaagcggaaagcatgcaagcttagttgcaagttcgttcctgagcacggggagcaacaacatgaagaagaagaaagagaaagagaacatccacaaccacacccaccacacgaggagagaggacgtagagaaagaggacgccaggaaggtgagaaggaggaaaaggaacaagacgagcgtgaattacaaccacgtccatttccacgaccagaggagcatgaaggagaacagtggaaaggaaggcatcgtcatgaagacccggaagagagggcgagaatgagactgagggaagcagaaagaatagagagggaaagggaggaacagagggtgagggaggaggaagagaaactccggcgacgtgagaagaaggaacaacgggaagagaaagaagaagaaggtcaaggttcggaagactcacactcaaaaagacagaacaacccttttcacttcagctctaacaggttccaaacgctcttcaagaaccaacatggtcaccttcgcgtcctccagagattcgaccaacgctccccacaacttgagaaccttcgagactaccgtgttgtagagctcatggctaaacccaacaccctctttctcccccaccacgctgacgctgattttctcctccttgtccttagcggttagtacacttaactaattactactactaattaaacatcgatcatcactttgcttctgctcttagtactagttaatggtaatcaattttactaattacagggagagccttaattaacttggtgaaacccgacgacagagacccctactaccttgaccgtggttatgcacaaagaatccctgcaggaaccactgtctatttggttaacccggacaagaagaaagatctcagagtgattaaactcgccatacccgttaacaaacctggcaattttgaggtactgcatgcctttttcattatgacagattagttaattaattcttctaattaatttatttccaaatgtgaagtgaatatcaatctttgtccttttctctttctctaggatttcttcctatctagcactcaagaccagcaatcctacttgcaaggcttcagcgagaatattctggaggcttccttcaatgtaagcaagcaacacatgcaccatctaatttgcatttccgtttagtggtttgtctaaatgtcatactaattaaatattatcattatatttgcttgtgtgcgcaattggttttatagaccaaattcgaggagataaacagggttttgtttggtggagaggggaggcggcaccagcaagagggagtgattttggaactatcaaaggaacagattcgggaactgagcaagcgtgccaaatctagtttcaaggagtactaattccttcgattatgaaccattctacttgagaggctcccaaatctcttccaacaactttggaaagttctatgagatcacccctgagaagaacccccagcttcgggactttgatatcctcctcaatactgtggatatcaacgaggtaagtgcaaaaaccatgtagatctagacaaagtgaactgcactattgagttctattcactctcttcttaattagttagttatactaattattgagactaaacaaatacagggaggtcttcttctgccacactacaattcaaaggccatagtgatactaatggttactgaaggagaagcaaacattgaactcgttggcctaaaagaacagcaacagggagaggaaacaagggaagtgcgaaagtatagagccgagttgtctgaagacgatatatttgtaatcccagcagcttatccatttgtggtcaacgctacctcaaacctcaatttcgttgcttttggtatcaatgctgagaacaaccagaacttccttgcaggtactatacatatataatattgattattgaataccctgttgttactttttgttgttttatggcatgtggaagcatgctgattgggttatttaattatttccaattcttggtgcgtgtgattgaaaatttgaaggttcgaaagacaatgtgataagacagatacaaaaacaggtgaaggagcttgcgttccctgctgggtctgcacaagatatcgagaatctaataaagaaccagagggaatcctattttgctgatgctcagcctttgcaaaaggaggaagggaagaaagggtcctttgtcttttgaatatgtatgcttaggagactaggagttcacagcgagcgaggaaagtatagggatatgtaactagaataacttgagatccacctttctactactagtaaataaaataaataaatcgag

SEQ ID NO: 85 - DNA, 2158 bp, synthetic construct
  source 1..2158: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatctagtgaacttccttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 86 - DNA, 2721 bp, synthetic construct
  source 1..2721: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcatgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagagttccttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 87 - DNA, 2720 bp, synthetic construct
  source 1..2720: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcatgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagattccttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 88 - DNA, 2718 bp, synthetic construct
  source 1..2718: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatctagtttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcatgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagagttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 89 - DNA, 2721 bp, synthetic construct; identical to SEQ ID NO: 86
  source 1..2721: mol_type = other DNA

SEQ ID NO: 90 - DNA, 2715 bp, synthetic construct
  source 1..2715: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcatgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagagttccttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 91 - DNA, 2724 bp, synthetic construct
  source 1..2724: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttaatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttgcttaattgtattcatttgatgcatggtaatgaattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaatgcatgtcacactaattaaatattaaaatgatagagcgaattcgaggagataaacagggttttgtttggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcatgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagggaacttccttgcaggtactatacatacactgttactactttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctctacctatcttctatctactacgaataaataaatccagcatacgatgcttgtgttttgtagccttcaactgttcaactgtcttcatatatatgcatggttggcacgtactcatatatatgctaaagtaatttacacgctcaagctaaattatgtgaggagtgtattaaacgaaaaaaaaaaagacataaattagagacatcttcagcaaagtgttacggtctaccagaaaagaaattgactttgttttcaagtcttccagactaagaagatcttgagtctaaatacttctaagaggtatcacgttgttgttttattgatgtatcatgtagtttgtgttttctggcctgctgctttaatttattttcttatatcatggctgatgtggctggctgtcgggaatatttggctactagatacatgtttaatggcagagttttgtatgtaaatggccactgtaggtgtaaaggttgatgtaggaatttaccaagggtagaaggcaggatactatgtatatgggttgggatatccttgtacctatttataaaatatattttaatatgtgctgatg

SEQ ID NO: 92 - DNA, 2310 bp, synthetic construct
  source 1..2310: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggagtgatcgtggaactctcaaaggaacaaattcggcaactgagcagacgtgccaaatctagttcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcgtgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagaggaacttccttgcaggtactatacatacccgttacggctttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctccacctatcttctatctactacgaataaataaatccagcatatgatgcttgtgttttgtagccttcaactgttcaactgtcttcatatatatgcatggttggcacgtactcatatatatgctaaagtaatttacccgctcaagctaaattatctg

SEQ ID NO: 93 - DNA, 1923 bp, synthetic construct
  source 1..1923: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggagtgatcgtggaactctcaaaggaacaaattcggcaactgagcagacgtgaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcgtgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagaggaacttaacttgagctccacctatcttctatctactacgaataaataaatccagcatatgatgcttgtgttttgtagccttcaactgttcaactgtcttcatatatatgcatggttggcacgtactcatatatatgctaaagtaatttacccgctcaagctaaattatctg

SEQ ID NO: 94 - DNA, 2301 bp, synthetic construct
  source 1..2301: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggagtgatcgtggaactctcaaaggaacaaattcggcaactgagcaggcgtgccaaatcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcgtgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagagttccttgcaggtactatacatacactgttactgctttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctccacctatcttctatctactacgaataaataaatccagcatatgatgcttgtgttttgtagccttcaactgttcaactgtcttcatatatatgcatggttggcacgtactcatatatatgctaaagtaatttacccgctcaagctaaattatctg

SEQ ID NO: 95 - DNA, 2299 bp, synthetic construct
  source 1..2299: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggagtgatcgtggaactctcaaaggaacaaattcggcaactgagcagacgtgccaaaaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcgtgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagagttccttgcaggtactatacatacactgttactgctttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctccacctatcttctatctactacgaataaataaatccagcatatgatgcttgtgttttgtagccttcaactgttcaactgtcttcatatatatgcatggttggcacgtactcatatatatgctaaagtaatttacccgctcaagctaaattatctg

SEQ ID NO: 96 - DNA, 1160 bp, synthetic construct
  source 1..1160: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggtgtgatcatgatacatagttca

SEQ ID NO: 97 - DNA, 2303 bp, synthetic construct
  source 1..2303: mol_type = other DNA
gtccaataatatatatactatgatgagagtgcggtttcctttgttggtgttgctgggaactgttttcctggcatcagtttgtgtctcattaaaggtgagagaggatgagaataaccctttctacttgagaagctctaacagcttccaaactctctttgagaaccaaaacggtcgcattcgtctcctccagagattcaacaaacgctccccacaacttgagaaccttcgagactaccggattgtccagtttcagtcaaaacccaacacaatccttctcccccaccatgctgacgccgatttcctcctctttgtccttagcggtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacagggagagccatacttaccttggtgaacaacgacgacagagactcctacaaccttcaccctggcgatgcccagagaatcccagctggaaccacttactatttggttaaccctcacgaccaccagaatctcaaaataatcaaacttgccatacccgtcaacaaacctggcagatatgatgtatgcacttctctttctctctttatatataacacattcttccattcttcttcttctaattatttatttatttatttccaaaatgtacgtgatgaccatcaattttaatttgtcctttctctctctgtaggatttcttcttatctagcactcaagcccaacagtcctacttgcaaggcttcagccataatattctagagacctccttccatgtaataagcaaccaacagtactctatctaactatttgcattcccatttagagattagtctaaattcatgtcacactaattaaatattaaaatgatagagcgaattcgaggagataaacagggttttgttgggagaggaagaggagcagaggcagcaagagggagtgatcgtggaactctcaaaggaacaaattcggcaactgagcagacgtgccaaatctagtcaaggaaaaccatttcctccgaagatgaaccattcaacttgagaagccgcaaccccatctattccaacaactttggaaagttctttgagatcacccctgagaaaaacccacagcttcgggacttggatatcttcctcagttctgtggatatcaacgaagtaagcataaaattaagaaaaaccatatactcaaactcgtgaactggacaattcagttctattcactctctccttagttagcgaacctaattattcagttctatcaacaaataatgatttcttcttttgttcacaaatatagggagctcttcttctaccacacttcaattcaaaggccatagtgatactagtgattaatgaaggagatgcaaacattgaacttgttggcattaaagaacaacaacagaagcagaaacaggaagaggaacctttggaagtgcaaaggtacagagctgaattgtctgaagacgatgtatttgtaattccagcagcttatccatttgtcgtcaacgctacctcaaacctcaatttccttgcttttggtatcaatgctgagaacaaccagagcttgcaggtactatacatacactgttactgctttttttgcatgtgaaacattagctgatttggttatttatttccaattctttggtgcctgattgaaaatttgaaggcgagaaagacaatgtggtaaggcagatagaaagacaagtgcaggagcttgcgttccctgggtctgcacaagatgttgagaggctattaaagaagcagagggaatcctactttgttgatgctcagcctcagcagaaggaggaggggagtaagggaagaaagggtccttttccttcaatcttaggtgctctctactgaataactacgtaaatgtatgctgtaagagctcatcgtgagcgtggaatgtatctgcccatgtaactataaaaacttgagctccacctatcttctatctactacgaataaataaatccagcatatgatgcttgtgttttgtagccttcaactgttcaactgtcttcatatatatgcatggttggcacgtactcatatatatgctaaagtaatttacccgctcaagctaaattatctg

SEQ ID NO: 98 - DNA, 2951 bp, synthetic construct
  source 1..2951: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctagtttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagagacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 99 - DNA, 3036 bp, synthetic construct
  source 1..3036: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 100 - DNA, 3034 bp, synthetic construct
  source 1..3034: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 101 - DNA, 3031 bp, synthetic construct
  source 1..3031: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaatcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 102 - DNA, 3038 bp, synthetic construct
  source 1..3038: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctagtctctggttgttctcggcattaataccaatagcaaagaaattcagatttgaggtagcgttgaccacaactggataacctgctgggattacaaatatatcttgttcagacaattcggctctatatttccgcacttccaaaggttgctcttcctgttgctgctcctgttgttgttcttttaggccaacaagttcaatgtttgcatctccttcattaattaccagtatcactatcgcctttgaattgaagtgtggtagaagaagagctccctatatttgtgaacaaagaaaaaagtaaatcattatttaaagtctcaataataggcttctctaactaagaagagagtgaatagtagaactcaattgtcctgttcagtttgtctacatggtttttctgcttacctcgttcatatccacaatactgaggaagatatccaagtcccgaagctgggggtttttctctggggtgatctcaaagaacttgccaagcttgttggagtagatggggtcgcggcttctcaagttaaaaggtttatcttcagaagaaatggttttccttgagaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 103 - DNA, 3034 bp, synthetic construct
  source 1..3034: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 104 - DNA, 2814 bp, synthetic construct
  source 1..2814: mol_type = other DNA
agcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatcttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagagaaaacaaccagagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacagcatatgatgcttttgttttgtgt

SEQ ID NO: 105 - DNA, 3100 bp, synthetic construct
  source 1..3100: mol_type = other DNA
agtggacccaaaagccatgcacaacaacacgtactcaccaaggtgcaatcgtgctgcccaaaaacattcaccaactcaatccatgatgagcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagagaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacag

SEQ ID NO: 106 - DNA, 3099 bp, synthetic construct
  source 1..3099: mol_type = other DNA
agtggacccaaaagccatgcacaacaacacgtactcaccaaggtgcaatcgtgctgcccaaaaacattcaccaactcaatccatgatgagcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatctaaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacag

SEQ ID NO: 107 - DNA, 3099 bp, synthetic construct
  source 1..3099: mol_type = other DNA
agtggacccaaaagccatgcacaacaacacgtactcaccaaggtgcaatcgtgctgcccaaaaacattcaccaactcaatccatgatgagcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacag

SEQ ID NO: 108 - DNA, 3100 bp, synthetic construct
  source 1..3100: mol_type = other DNA
agtggacccaaaagccatgcacaacaacacgtactcaccaaggtgcaatcgtgctgcccaaaaacattcaccaactcaatccatgatgagcccacacatttgttgtttgtaaccaaatctcaaacgcggtgttctctttggaaagcaaccatatcagcatatcacactatctagtctcttggatcatgcatgcgcaaccaaaagacaacacataaagtatcctttcgaaagcaatgtccaagtccatcaaataaaattgagacaaaatgcaacctcaccccacttcactatccatggctgatcaagatcgccgcgtccatgtaggtctaaatgccatgcacatcaacacgtactcaacatgcagcccaaattgctcaccatcgctcaacacatttcttgttaatttctaagtacactgcctatgcgactctaactcgatcacaaccatcttccgtcacatcaattttgttcaattcaacacccgtcaacttgcatgccaccccatgcatgcaagttaacaagagctatatctcttctatgactataaatacccgcaatctcggtccaggttttcatcatcgagaactagttcaatatcctagtataccttaataaataatttaagatactatgatgagagcacggttcccattactgttgctgggacttgttttcctggcttcagtttctgtctcatttggcattgcttactgggaaaaagagaaccccaaacacaacaagtgtctccagagttgcaatagcgagagagactcgtacaggaaccaagcatgccacgctcgttgcaacctccttaaggtggagaaagaagaatgtgaagaaggtgaaattccacgaccacgaccacgaccacaacacccggagagggaacctcagcaacccggtgagaaggaggaagacgaagatgagcaaccacgtccaatcccattcccacgcccacaacctcgtcaagaagaagagcacgagcagagagaggaacaggaatggcctcgcaaggaggaaaaacgcggagaaaagggaagtgaagaggaagatgaggatgaggatgaggaacaagatgaacgtcaattcccattcccacgcccacctcatcagaaggaagagcgaaagcaagaggaagatgaggatgaggagcagcagcgagagagcgaagaaagtgaagattctgagttacgaagacataagaataagaacccttttctcttcggctctaacaggttcgaaactctcttcaaaaaccaatatggtcgcattcgcgtcctccagaggttcaaccaacgctccccacaacttcagaatctccgagactaccgcattttggagttcaactccaaacccaacaccctccttctccccaaccatgctgacgctgattacctcatcgttatccttaacggtgagtattatttacttaataaacatcatcacttgcttctgcttcttgtaaaattatgatgatgaatttgttctgttaattacagggactgccattctttccttggtgaacaacgacgacagagactcctacagacttcaatctggtgatgccctgagagtcccctcaggaaccacatactatgtggtcaaccctgacaacaacgaaaatctcagattaataacactcgccatacccgttaacaagcctggtagatttgaggtactgccctttctttataaataattaattattcttctaatttgtttccaaatttgaagatcaatcttcctttttttcccctgtagagtttcttcctatctagcactgaagctcaacaatcctacttgcaaggattcagcaggaacattttagaggcctcctacgatgtaagaaaccaacacaccatctaattttcattaccatttagtagtttatccaactgtcacactacactggctaattaaatattatcgttatccttatatatttgtttccgtgcttgattttacagaccaaattcgaggagataaacaaggttctgtttagtagagaggaagggcagcagcaaggggagcagaggctgcaagagagcgtgattgtggaaatctcgaaggaacagattcgggcactgagcaaacgtgccaaatcttcaaggaaaaccatttcttctgaagataaaccttttaacttgagaagccgcgaccccatctactccaacaagcttggcaagttctttgagatcaccccagagaaaaacccccagcttcgggacttggatatcttcctcagtattgtggatatgaacgaggtaagcagaaaaaccatgtagacaaactgaacaggacaattgagttctactattcactctcttcttagttagagaagcctattattgagactttaaataatgatttacttttttctttgttcacaaatatagggagctcttcttctaccacacttcaattcaaaggcgatagtgatactggtaattaatgaaggagatgcaaacattgaacttgttggcctaaaagaacaacaacaggagcagcaacaggaagagcaacctttggaagtgcggaaatatagagccgaattgtctgaacaagatatatttgtaatcccagcaggttatccagttgtggtcaacgctacctcaaatctgaatttctttgctattggtattaatgccgagaacaaccagaggaacttcctcgcaggtatataatttatatatcacaatcaaatagaatattgttgttacttttttgttttattgcatgtgaaacatagccgcctgagttatttcaaacctaaacttggtgattgaaaacttgaaggttcgcaagacaatgtgataagccagatacctagtcaagtgcaggagcttgcattccctgggtctgcacaagctgttgagaagctattaaagaaccaaagagaatcctactttgtggatgctcagcctaagaagaaagaggaggggaataagggaagaaagggtcctttgtcttcaattttgagggctttttactgaataagtatgtactaaaatgtatgctgtaatagctcatagtgagcgaggaaagtatcgggctatgtaactatgactagagcttcaactatgaataaataaatcgacag

SEQ ID NO: 109 - DNA, 19 bp, synthetic construct
  source 1..19: mol_type = other DNA
acgtgccaaatctagttca

SEQ ID NO: 110 - DNA, 19 bp, synthetic construct
  source 1..19: mol_type = other DNA
acctgcaaggaagttcctc

SEQ ID NO: 111 - DNA, 19 bp, synthetic construct
  source 1..19: mol_type = other DNA
catgcaacgaagatagcac

SEQ ID NO: 112 - DNA, 19 bp, synthetic construct
  source 1..19: mol_type = other DNA
ggacaacaaacaccattcc

SEQ ID NO: 113 - DNA, 19 bp, synthetic construct
  source 1..19: mol_type = other DNA
ctgcagttgcggcgcgcct

SEQ ID NO: 114 - DNA, 19 bp, synthetic construct
  source 1..19: mol_type = other DNA
ctacaattgcggcgcgcct

SEQ ID NO: 115 - DNA, 24 bp, synthetic construct
  source 1..24: mol_type = other DNA
acctggcagatatgatgtatgcac

SEQ ID NO: 116 - DNA, 23 bp, synthetic construct
  source 1..23: mol_type = other DNA
gacagccagccacatcagccatg

SEQ ID NO: 117 - DNA, 24 bp, synthetic construct
  source 1..24: mol_type = other DNA
acctggcagatatgatgtatgcac

SEQ ID NO: 118 - DNA, 23 bp, synthetic construct
  source 1..23: mol_type = other DNA
gaactatgtatcatgatcacacc

SEQ ID NO: 119 - DNA, 23 bp, synthetic construct
  source 1..23: mol_type = other DNA
tgatgccctgagagtcccctcag

SEQ ID NO: 120 - DNA, 24 bp, synthetic construct
  source 1..24: mol_type = other DNA
cttcagagtaaattacttctgcgt

SEQ ID NO: 121 - DNA, 24 bp, synthetic construct
  source 1..24: mol_type = other DNA
ctaccgtgttgtagagctcatggc

SEQ ID NO: 122 - DNA, 24 bp, synthetic construct
  source 1..24: mol_type = other DNA
acccaatcagcatgcttccacatg

SEQ ID NO: 123 - DNA, 23 bp, synthetic construct
  source 1..23: mol_type = other DNA
cgtgatctgggactgatcttgac

SEQ ID NO: 124 - DNA, 22 bp, synthetic construct
  source 1..22: mol_type = other DNA
acattccccgtgatcttcagcc

SEQ ID NO: 125 - DNA, 23 bp, synthetic construct
  source 1..23: mol_type = other DNA
cttattgtatggatggtatagtc

SEQ ID NO: 126 - DNA, 22 bp, synthetic construct
  source 1..22: mol_type = other DNA
tgtgtactcctacctgcaaacg

SEQ ID NO: 127 - DNA, 24 bp, synthetic construct
  source 1..24: mol_type = other DNA
atccaactgtcacactacactggc

SEQ ID NO: 128 - DNA, 23 bp, synthetic construct
  source 1..23: mol_type = other DNA
aagcatcatatgctgtcctgtcg

SEQ ID NO: 129 - DNA, 24 bp, synthetic construct
  source 1..24: mol_type = other DNA
gcgatgccctaagagtccctgcag

SEQ ID NO: 130 - DNA, 23 bp, synthetic construct
  source 1..23: mol_type = other DNA
tagctcagtcagctatgtttcac

SEQ ID NO: 131 - DNA, 22 bp, synthetic construct
  source 1..22: mol_type = other DNA
gacaaaggagtgacgcgtcacg

SEQ ID NO: 132 - DNA, 24 bp, synthetic construct
  source 1..24: mol_type = other DNA
caacatatgaagcagtttccatgc

SEQ ID NO: 133 - DNA, 24 bp, synthetic construct
  source 1..24: mol_type = other DNA
accccatcgcaaatcgaagagtac

SEQ ID NO: 134 - DNA, 23 bp, synthetic construct
  source 1..23: mol_type = other DNA
gacgtcctcggcggcggcgaggt

(19) United States
(12) Patent Application Publication
(10) Pub. No.: US 2025/0283104 A1
(43) Pub. Date: Sep. 11, 2025

(54) METHODS AND COMPOSITIONS FOR INCREASING AMINO ACID AND PROTEIN CONTENT IN PLANTS

(71) Applicant: THE CURATORS OF THE UNIVERSITY OF MISSOURI, Columbia, MO (US)

(72) Inventors: Minviluz G. Stacey, Columbia, MO (US); Cuong X. Nguyen, Columbia, MO (US)

(21) Appl. No.: 18/861,126

(22) PCT Filed: May 2, 2023
(86) PCT No.: PCT/US2023/066484

§ 371 Date: Oct. 28, 2024

Related U.S. Application Data
(60) Provisional application No. 63/363,988, filed on May 2, 2022.

(51) Int. Cl. C12N 15/82; A23K 10/30; C07K 14/415; C12N 9/10; C12N 9/22; C12Q 1/6895

(52) U.S. Cl. CPC C12N 15/8251; A23K 10/30; C07K 14/415; C12N 9/1085; C12N 9/222; C12N 15/8201; C12Q 1/6895; C12N 2310/20; C12Q 2600/13; C12Y 205/01048

(57) ABSTRACT

Plant seeds with increased protein and methionine content and having a modified expression of a cystathionine γ-synthase (CGS) polypeptide, modified expression of a low methionine content (< about 1.0% methionine) storage protein polypeptide, or modified expression of both are provided. Methods for modifying expression of CGS polypeptides and polynucleotides and low methionine content seed storage polypeptides and polynucleotides include genome editing to modify the MTO1 regulatory region of CGS to create feedback insensitive methionine production and low methionine content polypeptides to create proteome rebalancing toward high methionine content storage protein production, and transformation with recombinant DNA constructs to enhance or suppress expression are disclosed herein.

### CROSS REFERENCE TO RELATED APPLICATIONS

This application claims priority to provisional application U.S. Ser. No. 63/363,988, filed May 2, 2022, which is incorporated herein by reference in its entirety.

### SEQUENCE LISTING XML

The instant application contains a sequence listing, which has been submitted in XML file format by electronic submission and is hereby incorporated by reference in its entirety. The XML file, created on Apr. 28, 2023, is named P13867WO00.xml and is 365,279 bytes in size.

### TECHNICAL FIELD

The present disclosure generally relates to the field of biotechnology. More specifically, the present disclosure relates to increasing amino acid content in plants, particularly methionine.

### BACKGROUND

Human and monogastric mammals are unable to synthesize essential amino acids that are required for survival, such as methionine. Hence, they have to obtain them from their diets, mainly from plant-based resources. Deficiency of these amino acids can lead to protein-energy malnutrition (PEM) syndrome which shows some nonspecific signs of protein deficiencies such as lowered resistance to diseases, decreased blood proteins, and retarded mental and physical development.

### SUMMARY

Provided are plants, seeds, plant parts, and plant cells and descendants thereof that have a genomic modification that inhibits the feedback regulation cystathionine γ-synthase (CGS) catalyzed methionine production and/or a genomic modification that decreases expression of low methionine storage proteins. The genomic modification can be a deletion, insertion, or substitution of one or more nucleotides in a genomic sequence encoding a CGS or a low methionine content storage protein which modification suppresses the activity of the feedback regulation by MTO1 of the CGS and/or that suppresses the production of low methionine content storage proteins to cause proteome rebalancing favoring production of high methionine content storage proteins.

In some embodiments, the low methionine content storage protein is a beta-conglycinin and the high methionine storage protein is a glycinin. In some embodiments, the genomic modification suppresses the activity or production of 7S storage protein (e.g., an alpha′, alpha, or beta subunit of the 7S storage protein). In some embodiments, the modification in the plant comprises a deletion or insertion in a coding sequence of the polypeptide, which may result in a frameshift of the sequence encoding the low methionine storage polypeptide. In some embodiments, the modification comprises a deletion, insertion, or substitution which may result in an in-frame modification of the sequence encoding the MTO1 feedback regulatory region of CGS which retains enzymatic activity.

Plants producing seeds and seeds are provided, which have an increased total methionine and protein content of up to 30% and 3%, respectively, in seeds compared to seeds not comprising the modification.

In some embodiments, plants are provided which further include a heterologous nucleic acid sequence such as one or more of a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

Methods are provided for increasing protein content in the seed of a plant, by introducing a modification into a CGS gene within or near the MTO1 region and/or a low methionine content storage polypeptide gene, or both genes in a plant. Examples of modifications include a deletion, insertion, or substitution of one or more nucleotides in a genomic sequence encoding a CGS feedback regulatory region and/or a low methionine storage polypeptide comprising an amino acid sequence as disclosed herein to produce a modified plant. The modified plant can be grown to produce a modified seed, which has an increased protein content or increased methionine content compared to seed of a control plant not comprising the modification.

In some embodiments, methods of plant breeding include crossing the plants provided with a second plant to produce progeny seed. Progeny seed produced by these methods may comprise the modification and have increased protein content or increased methionine content relative to a control seed not comprising the modification.

In some embodiments, the modification comprises a deletion or insertion in a coding sequence of the polypeptide, which may result in a frameshift of the sequence encoding the low methionine storage polypeptide. In some embodiments, the modification comprises a deletion or insertion in a coding sequence of the CGS feedback regulatory region of the CGS polypeptide, which may result in an in-frame modification that inhibits feedback regulation of the CGS polypeptide. The deletion or insertion may be introduced through targeted DNA breaks.

Provided are methods for producing a plant producing a seed comprising increased protein content or increased methionine content, by introducing into a plant a recombinant DNA construct comprising a heterologous polynucleotide, the polynucleotide comprising a sequence that results in reduced expression of a low methionine content seed storage and/or a reduced feedback regulation of CGS activity, or a combination thereof and regenerating a plant producing a seed comprising increased protein content or increased methionine content. In some embodiments, the reduced expression of the polypeptide is achieved through gene silencing or RNAi. Plants producing seeds and seeds produced by this method are provided, in which the plant or seed comprises the heterologous polynucleotide stably incorporated into its genome, and the seed has an increased protein content or increased methionine content compared to control seeds not comprising the heterologous polynucleotide. In some embodiments, the seed or progeny seed produced by the plant has seed methionine content that is increased by about 30% compared to a control seed not comprising the heterologous polynucleotide.

Provided are guide RNA sequences, and recombinant DNA constructs expressing the guide RNAs, that target a genomic locus of a plant cell. Soybean plant cells, plants and seeds comprising the guide RNA sequence are provided. In some embodiments, the guide RNA comprises the nucleotide sequence of SEQ ID NO: 109, 110, 111, 112, 113, or 114. Seeds, such as produced by the plant or regenerated from the plant cell have an increased protein content or increased methionine content compared to control seeds not comprising the guide RNA sequence.

Provided are methods of detecting the presence of a polynucleotide comprising a sequence disclosed herein, by contacting a DNA sample obtained from a plant which comprises the modifications disclosed herein with a first primer molecule which binds to a genomic region upstream of the modification or at least partially contained in the modification and a second primer molecule which binds to a genomic region downstream of modification or at least partially contained in the same. A nucleic acid amplification reaction condition is provided and performed to produce a DNA amplicon molecule, such as 48 to 5000 nucleotides in length, which indicates the presence of the modification and which is detected.

While multiple embodiments are disclosed, still other embodiments will become apparent to those skilled in the art from the following detailed description, which shows and describes illustrative embodiments. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A-H shows increased free Met in leaves and seed of the Arabidopsis in-frame MTO1 CRISPR mutant lines. FIG. 1A are photographs showing the growth of 10-day-old seedling of wild-type (Col-0), mto1-1, and two CRISPR lines (At_CRS3-2 and At_CRS4-2) on ½ MS media (upper panel) and ½ MS media+100 μM ethionine (lower panel), Scale bar=1 cm. FIG. 1B shows an alignment of DNA sequences of the MTO1 region in wild-type, mto1-1, and the two CRISPR lines (SEQ ID NOs: 31-36). The PAM sequence is underlined, and the point mutation of mto1-1 is indicated. Mutant amplicons showed −39 bp and −42 bp deletions due to excision of DNA sequences near the PAM site. FIG. 1C shows PCR-based genotyping of wild-type (Col-0), mto1-1, and the two CRISPR lines. Higher mobility amplicons indicate CRISPR/Cas9-induced deletion in AtCGS1 in the two CRISPR mutant lines. FIG. 1D shows the relative expression level of AtCGS1 from leaves of two weeks old seedling on ½ MS media. Data represents mean±SD, n=4. Statistical analysis was performed using one-way ANOVA followed by a post-hoc Tukey's multiple range test. Different letters denote significant differences at P<0.001. FIG. 1E-G shows free Met content in leaves, siliques, and seed; FIG. 1H shows total Met content in seeds. Data represents #SD, n=5. Statistical analysis was performed using one-way ANOVA followed by a post-hoc Tukey's multiple range test. Different letters denote significant differences at P<0.01.

FIG. 2A-C is a schematic of the CRISPR/Cas9 targets in soybean. FIG. 2A shows the location of the target in the MTO1 region of GmCGS1 and GmCGS2 (SEQ ID NOs: 37-38). Gene-specific primers used for polymerase chain reaction (PCR) genotyping are indicated. The PAM sites are underlined, and the AscI restriction sites are shown as an arrow. FIG. 2B-C shows the location of targets in the 7S genes (SEQ ID NOs: 39-42). Gene-specific primers used for polymerase chain reaction (PCR) genotyping are indicated and the PAM sites are underlined.

FIG. 3A-L shows mutations within the MTO1 region of the GmCGS genes lead to changing free Met in leaves and seed in soybean. FIG. 3A are photographs showing the growth of 10 days old seedling of wild-type (Maverick), and CRISPR mutant lines 7SKO-1, 7SKO-2, 7SKO/cgs1KO-1, 7SKO/cgs1KO-2, 7SKO/cgs2KO, 7SKO/cgs1inf-1, 7SKO/cgs1inf-2, 7SKO/cgs2inf-1, 7SKO/cgs2inf-2 and 7SKO/cgs1inf/cgs2inf on ½ MS media (upper panel) and ½ MS media+100 μM ethionine (lower panel). FIG. 3B shows quantification of root length of wild-type and the CRISPR mutant lines. Data represents mean±SD, n>15 and n>22 for control and ethionine treatment, respectively. Statistical analysis was performed using one-way ANOVA using genotype as a factor followed by a post-hoc Tukey's multiple range test. Different letters denote significant differences at p<0.001 among genotypes under the same treatment. FIG. 3C shows DNA sequence alignments of MTO1 region within the GmCGS1 (Glyma.09g235400) and GmCGS2 (Glyma.18g261600) genes in wild-type and four T0 transgenic lines: 7S-CGS-4, 7S-CGS-6, 7S-CGS-13, 7S-CGS-36 (SEQ ID NOs: 42-51). The PAM sequence is underlined. The sequence was cleaved by AscI (arrows indicate the cutting site). Mutant amplicons showed deletions (−) or insertions (+) near the PAM site. FIG. 3D-E shows CAPS (Cleaved Amplified Polymorphic Sequences) marker genotyping of wild-type (WT) and the T6 CRISPR mutant lines [7SKO-1 (1), 7SKO-2 (2), 7SKO/cgs1KO-1 (3), 7SKO/cgs1KO-2 (4), 7SKO/cgs2KO (5), 7SKO/cgs1inf-1 (6), 7SKO/cgs1inf-2 (7), 7SKO/cgs2inf-1 (8), 7SKO/cgs2inf-2 (9), and 7SKO/cgs1inf/cgs2 (10)] to detect mutations in GmCGS1 and GmCGS2 before (upper panel) and after (lower panel) AscI digestion. FIG. 3F-I shows relative expression level of GmCGS1 and GmCGS2 from leaves (FIG. 3F-G) of four weeks old plants and green seeds (FIG. 3H-I). Data represents mean±SD, n=4. Statistical analysis was performed using one-way ANOVA followed by a post-hoc Tukey's multiple range test. Different letters denote significant differences at P<0.01.

FIG. 3J-L shows free Met content in roots, leaves, green seeds, and mature seed. Data represents mean±SD, n=5. Statistical analysis was performed using one-way ANOVA followed by a post-hoc Tukey's multiple range test. Different letters denote significant differences at P<0.01.

FIG. 4A-F shows quantitative proteomic analysis of soybean seeds of different genotypes. FIG. 4A shows qualitative overview and intersections of quantified proteins from the mature seed proteome of six soybean CRISRP/Cas9 lines and Maverick wild-type. Horizontal bars show the number of proteins found in the genotypes. Vertical bars display intersects between comparisons as indicated in the matrix below the graph. FIG. 4B-F shows protein abundance of 7S, 11S (glycinin), KTI, and BBI in the mature soybean seeds among seven genotypes. Data represents mean±SD, n=5. Statistical analysis was performed using one-way ANOVA followed by a post-hoc Tukey's multiple range test. Different letters denote significant differences at P<0.01.

FIG. 5A-D shows the increase in total methionine, amino acid, and protein in seed of the field grown soybean CRISPR/Cas9 lines. FIG. 5A-B shows total methionine and total amino acid content in dry mature seeds of the wild-type and CRISPR lines, planted in the Bradford Research and Experiment Center, University of Missouri in 2020 and 2021. Quantitative analysis of amino acids were performed with standard protein hydrolysate at the Agricultural Experiment Station Chemical Laboratories (ESCL), University of Missouri, Columbia. FIG. 5C-D shows protein content, measured by Combustion method (LECO) and oil content, measured by the Soxhlet method in wild-type (Maverick) and CRISPR/Cas9 lines. For all boxplots, the box represents the interquartile range, the horizontal line in the box is the median, red dot is the mean, n=5. Statistical analysis was performed using one-way ANOVA followed by a post-hoc Tukey's multiple range test. Different letters denote significant differences at P<0.01.

FIG. 6A-D shows germination rate and plant performance of the soybean lines. FIG. 6A are photographs showing soybean seed germination 5 days after sowing. The experiments were carried out in an incubator at 25° C., 75% humidity FIG. 6B-D shows 100 seed weight, plant height, and number of nodes per plant measured from field grown soybeans. Data represents mean±SD, n>15. Statistical analysis was performed using one-way ANOVA followed by a post-hoc Tukey's multiple range test. P<0.01.

### DETAILED DESCRIPTION

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out herein.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and includes any combination of members of that list. Further, all units, prefixes, and symbols may be denoted in its SI accepted form.

Compositions and methods related to modified plants producing seeds high in protein content are provided. Plants that have been modified using genomic editing techniques, transformation, or mutagenesis to produce seeds having increased protein or methionine are provided. Suitable plants include oil-seed plants, such as palm, canola, sunflower, and soybean as well as, without limitation, rice, cotton, sorghum, wheat, maize, alfalfa, and barley. Modifying expression of a CGS polypeptide and/or a low methionine content seed storage polypeptide in a plant, results in a seed with high protein relative to a comparable seed not comprising the modification. The modification can be introduced using genomic editing technology, transformation, or mutagenesis, such as described herein. Plants, such as soybean plants, that show reduced feedback regulation of CGS and/or reduced expression of a low methionine content seed storage protein or a combination thereof, do not show any detrimental plant growth or plant morphology characteristics are robust, high-yielding, and produce seeds containing increased protein are provided.

Unless specified otherwise, protein, fiber, stachyose, sucrosyl-oligosaccharide and other components are measured by weight at or adjusted to a 13% moisture basis in the seeds. Seeds, plants (or plant parts thereof) producing seeds, and methods of making or using the seeds and plants (or plant parts thereof) and having the seed compositions described herein are provided.

Provided are modified plant seeds and plants producing such seeds, as described herein, containing a substantially similar or increased protein content compared with a comparable unmodified, control, null or wild-type seed. The total content of methionine of the modified plant seed may be at least or at least about 15%, 16%, 17%, 18%, 19%, 19.5%, 20%, 20.5%, 21%, 21.5%, 22%, 22.5%, 23%, 23.5%, 24%, 24.5%, or 25% 26%, 27%, 28%, 29, or 30% greater than the comparable unmodified, control, null or wild-type plant. The seed protein content may be 0.1, 0.2, 0.3, 0.4, 0.5, 0.5, 0.7, 0.8. 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 percent greater when compared to the amount of protein in a comparable unmodified, control, null or wild-type seed.

The seeds can be efficiently processed to produce meal (either high-protein meal produced from dehulled seeds or conventional meal produced from whole seeds) having a high protein content compared with comparable meal produced from comparable seeds that do not contain the modification. In some embodiments, meal is provided which has a protein content that is increased by up to 3% by weight compared to meal prepared from a control soybean seed not comprising the modification, such as a null, unmodified or wild-type soybean seed. In some embodiments, meal is provided which has a total methionine content that is increased by up to 30% by weight compared to meal prepared from a control soybean seed not comprising the modification, such as a null, unmodified or wild-type soybean seed. The meal may be prepared from a plant comprising the modification and may comprise a modified polynucleotide described herein.

Provided are modified soybean seeds and plants, plant parts and plant cells which have an increased protein or methionine content and at least a comparable or increased yield, such as described herein, relative to a comparable control unmodified seed and plant, plant part or plant cell not comprising the modification.

The modified polypeptides and polynucleotides described herein include or encode feedback insensitive CGS polypeptides or low methionine content storage polypeptides, or a combination thereof. CGS polypeptides are a group of proteins that catalyze the first committed step of methionine biosynthesis in plants. The N-terminal extension of plant CGS possess a chloroplast transit peptide (cTP) and a MTO1 region for negative feedback regulation of CGS gene expression. Glycinins (11S) and β-conglycinins (7S) are two major classes of seed storage proteins which constitute approximately 33-40% and 21-30% of total seed protein, respectively. Met and Cys comprise 3.0-4.5% of glycinins amino acid residues; whereas less than 1.0% of Met and Cys is found in the 7S β-conglycinin fractions. Suppressing genes encoding for methionine-poor storage seed proteins showed accumulation of methionine-rich proteins through proteome rebalancing. Plants with higher 11S: 7S ratio were found to have higher total amino acid content in seed.

Table 1 provides a summary of wild-type soybean CGS polypeptide and low methionine content storage polypeptide sequences.

TABLE 1

| | Genomic | Coding | Amino acid |
|---|---|---|---|
| | sequence | sequence | sequence |
| CGS1 | SEQ ID NO: 1 | SEQ ID NO: 2 | SEQ ID NO: 3 |
| CGS2 | SEQ ID NO: 4 | SEQ ID NO: 5 | SEQ ID NO: 6 |
| 7S α subunit | SEQ ID NO: 7 | SEQ ID NO: 8 | SEQ ID NO: 9 |
| 7S α subunit | SEQ ID NO: 10 | SEQ ID NO: 11 | SEQ ID NO: 12 |
| 7S α′ subunit | SEQ ID NO: 13 | SEQ ID NO: 14 | SEQ ID NO: 15 |
| 7S α′ subunit | SEQ ID NO: 16 | SEQ ID NO: 17 | SEQ ID NO: 18 |
| 7S α′ subunit | SEQ ID NO: 19 | SEQ ID NO: 20 | SEQ ID NO: 21 |
| 7S β-subunit | SEQ ID NO: 22 | SEQ ID NO: 23 | SEQ ID NO: 24 |
| 7S β-subunit | SEQ ID NO: 25 | SEQ ID NO: 26 | SEQ ID NO: 27 |
| SBP1 (Sucrose | SEQ ID NO: 28 | SEQ ID NO: 29 | SEQ ID NO: 30 |
| Binding | | | |
| Protein 1) | | | |

Provided are plants, plant cell, plant parts and seeds which have had expression of a polypeptide or polynucleotide sequence that encodes the polypeptide suppressed, knocked out, decreased, or inhibited. Examples of polypeptides include those depicted herein. In some embodiments, soybean plants, seeds, plant cells and methods are provided in which expression of both the CGS feedback regulatory function and the low methionine content polypeptide is reduced or suppressed.

In some embodiments, the modification results in the suppression of the native CGS or low methionine content storage polypeptide or both. The genome is modified to knock-out, silence, reduce or suppress expression of the native gene or the native feedback regulatory region of the CGS or both, such as by disrupting the reading frame through insertion or deletion of one or more single bases or short or long sequences, introducing a sufficient number of SNPs to disrupt function or by modifying a transcription regulatory sequence in the transcription regulatory region to include for example repressor elements, repressor binding elements or disrupted promotor enhancer elements to reduce or prevent expression of the same. In the case of CGS the insertion or deletion is an in-frame insertion or deletion that suppresses the feedback regulation of the gene but not the catalytic activity of the protein encoded thereby through, for example, and an in-frame modification.

In some embodiments, the expression level of the seed storage polynucleotide or polypeptide in a tissue or organ of interest, such as the seed, seed endosperm, embryo, leaf, root or stalk, is less than 95, 90, 85, 80, 75, 70, 65, 60, 55, 50, 45, 40, 35, 30, 25, 20, 15, 10, 5, 4, 3, 2, or 1% of the expression level of the polynucleotide or polypeptide in a comparable control, unmodified or null tissue or organ of interest. In some embodiments, the expression level of the CGS polynucleotide or polypeptide in a tissue or organ of interest, such as the seed, seed endosperm, embryo, leaf, root or stalk, is greater than 95, 90, 85, 80, 75, 70, 65, 60, 55, 50, 45, 40, 35, 30, 25, 20, 15, 10, 5, 4, 3, 2, or 1% of the expression level of the polynucleotide or polypeptide in a comparable control, unmodified or null tissue or organ of interest. Plants producing seeds with increased protein as described herein are obtained.

In certain embodiments, plants, plant parts, and seeds comprising a loss-of-function mutation (e.g., a frameshift mutation) in at least one subunit of the 7S storage protein (beta-conglycinin) are provided. In certain embodiments, the loss-of-function mutation (e.g., a frameshift mutation) is in the coding sequence of one or more alpha′ subunits of the 7S storage protein, one or more alpha subunits of the 7S storage protein, or one or more beta subunits of the 7S storage protein. In certain embodiments, the loss-of-function mutation (e.g., a frameshift mutation) is in the coding sequence of one or more alpha′ subunits of the 7S storage protein, one or more alpha subunits of the 7S storage protein, and one or more beta subunits of the 7S storage protein. In certain embodiments, the loss-of-function mutation (e.g., a frameshift mutation) is in the coding sequence of each alpha′ subunit of the 7S storage protein, each alpha subunit of the 7S storage protein, or each beta subunit of the 7S storage protein. In certain embodiments, the loss-of-function mutation (e.g., a frameshift mutation) is in the coding sequence of each alpha′ subunit of the 7S storage protein, each alpha subunit of the 7S storage protein, and each beta subunit of the 7S storage protein. In certain embodiments, the plants, plant parts, and seeds comprise (i) a loss-of-function mutation (e.g., a frameshift mutation) in the coding sequence of each alpha′ subunit of the 7S storage protein, each alpha subunit of the 7S storage protein, and each beta subunit of the 7S storage protein; and (ii) a modification (e.g., an in-frame mutation) in the coding sequence of a cystathionine gamma-synthase that suppresses feedback regulation and retains enzymatic activity.

In some embodiments, the plant, plant cell, plant part or seed includes or expresses the sequences shown in SEQ ID NOs herein or any combination thereof, or sequences sharing a percent identity with such sequences. In some embodiments, the plant, plant cell, plant part or seed includes a recombinant DNA construct or molecule or suppression construct described herein which suppresses or reduces expression of the polypeptide. Transformation methods for producing such soybean plants, plant cells, plant parts or seeds are provided.

In some embodiments, the plant further includes a heterologous nucleic acid sequence selected from the group of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants. The heterologous nucleic acid may be introduced by backcrossing or transformation.

Provided are polynucleotides that have at least about or at least 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to a reference nucleotide sequence, such as a nucleotide sequence disclosed in the sequence listing herein, using one of the alignment programs described herein using standard parameters, as well as nucleotide substitutions, deletions, insertions, fragments thereof, and combinations thereof.

An “isolated polynucleotide” generally refers to a polymer of ribonucleotides (RNA) or deoxyribonucleotides (DNA) that is single- or double-stranded, optionally containing synthetic, non-natural or altered nucleotide bases, that is no longer in its natural environment and have been placed in a difference environment by the hand of man, for example in vitro. An isolated polynucleotide in the form of DNA may be comprised of one or more segments of cDNA, genomic DNA or synthetic DNA.

A “recombinant” nucleic acid molecule (or DNA) is used herein to refer to a nucleic acid sequence (or DNA) that is in a recombinant plant host cell. In some embodiments, an “isolated” or “recombinant” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A transcription regulatory element or sequence, or a regulatory element or sequence generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

In some embodiments, the modified polynucleotide includes a modified transcriptional enhancer sequence. An enhancer element is any nucleic acid molecule that increases transcription of a nucleic acid molecule when functionally linked to a promoter regardless of its relative position. An enhancer may be an innate element of the promoter or a heterologous element inserted to enhance the amount of promotor activity or tissue-specificity of a promoter.

Various enhancers may be used including introns with gene expression enhancing properties in plants (US Patent Application Publication Number 2009/0144863), the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used, each of which is incorporated by reference. The above list of transcriptional enhancers is not meant to be limiting. Any appropriate transcriptional enhancer can be used in the embodiments.

A repressor (also sometimes called herein silencer, repressor element, or repressor binding element) is defined as any nucleic acid molecule which inhibits the transcription when functionally linked to a promoter regardless of relative position.

“Promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

The termination region may be native with the transcriptional initiation region, may be native with the operably linked DNA sequence of interest, may be native with the plant host or may be derived from another source (i.e., foreign or heterologous to the promoter, the sequence of interest, the plant or any combination thereof).

The sequences include one or more contiguous nucleotides. “Contiguous nucleotides” is used herein to refer to nucleotide residues that are immediately adjacent to one another.

As used herein non-genomic nucleic acid sequence, nucleic acid molecule or polynucleotide refers to a nucleic acid molecule that has one or more changes in the nucleic acid sequence compared to a native or genomic nucleic acid sequence. In some embodiments, the change to a native or genomic nucleic acid molecule includes but is not limited to: changes in the nucleic acid sequence due to the degeneracy of the genetic code; optimization of the nucleic acid sequence for expression in plants; changes in the nucleic acid sequence to introduce at least one amino acid substitution, insertion, deletion and/or addition compared to the native or genomic sequence; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments, the non-genomic nucleic acid molecule is a synthetic nucleic acid sequence.

Provided are polypeptides having at least about or at least 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to polypeptides referenced in the sequence listing, as well as amino acid substitutions, deletions, insertions, fragments thereof, and combinations thereof. The term “about” when used herein in context with percent sequence identity means+/−0.5%. These values can be appropriately adjusted to determine corresponding homology of proteins considering amino acid similarity and the like.

In some embodiments, the sequence identity is against the full-length sequence of a polypeptide disclosed in the sequence listing. In some embodiments, the polypeptide retains activity or shows enhanced or reduced activity.

As used herein, the term “protein,” “peptide molecule,” or “polypeptide” includes those molecules that undergo modification, including post-translational modifications, such as, but not limited to, disulfide bond formation, glycosylation, phosphorylation or oligomerization.

The terms “amino acid” and “amino acids” refer to all naturally occurring L-amino acids.

Variants may be made by making random mutations or the variants may be designed. In the case of designed mutants, there is a high probability of generating variants with similar activity to the native polypeptide when amino acid identity is maintained in critical regions of the polypeptide which account for biological activity or are involved in the determination of three-dimensional configuration which ultimately is responsible for the biological activity. A high probability of retaining activity will also occur if substitutions are conservative. Amino acids may be placed in the following classes: non-polar, uncharged polar, basic, and acidic. Conservative substitutions whereby an amino acid of one class is replaced with another amino acid of the same type are least likely to materially alter the biological activity of the variant.

Alternatively, alterations may be made to the protein sequence of many proteins at the amino or carboxy terminus without substantially affecting activity. This can include insertions, deletions or alterations introduced by modern molecular methods, such as polymerase chain reaction (PCR), including PCR amplifications that alter or extend the protein coding sequence by inclusion of amino acid encoding sequences in the oligonucleotides utilized in the PCR amplification. Alternatively, the protein sequences added can include entire protein-coding sequences, to generate protein fusions. Such fusion proteins are often used to (1) increase expression of a protein of interest (2) introduce a binding domain, enzymatic activity or epitope to facilitate either protein purification, protein detection or other experimental uses (3) target secretion or translation of a protein to a subcellular organelle, such as the periplasmic space of Gram-negative bacteria, mitochondria or chloroplasts of plants or the endoplasmic reticulum of eukaryotic cells, the latter of which often results in glycosylation of the protein.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions).times. 100). In one embodiment, the two sequences are the same length. In another embodiment, the percent identity is calculated across the entirety of the reference sequence. The percent identity between two sequences can be determined using techniques like those described below, with or without allowing gaps. In calculating percent identity, typically exact matches are counted. A gap, (a position in an alignment where a residue is present in one sequence but not in the other) is regarded as a position with non-identical residues.

The determination of percent identity between two sequences can be accomplished using a mathematical algorithm. A non-limiting example of a mathematical algorithm utilized for the comparison of two sequences is the algorithm incorporated into the BLASTN and BLASTX programs. Karlin and Altschul (1990) Proc. Nat'l. Acad. Sci. USA 87:2264, Altschul et al. (1990) J. Mol. Biol. 215:403, and Karlin and Altschul (1993) Proc. Nat'l. Acad. Sci. USA 90:5873-5877. BLAST nucleotide searches can be performed with the BLASTN program, score=100, word length=12, to obtain nucleotide sequences homologous to nucleic acid molecules disclosed herein. BLAST protein searches can be performed with the BLASTX program, score=50, word length=3, to obtain amino acid sequences homologous to polypeptides disclosed herein. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4 (1): 11-17. Such an algorithm is incorporated into the ALIGN program (version 2.0), which is part of the GCG Wisconsin Genetics Software Package, Version 10 (available from Accelrys, Inc., San Diego, Calif., USA). When utilizing the ALIGN program for comparing amino acid sequences, a PAM120 weight residue table, a gap length penalty of 12, and a gap penalty of 4 can be used. Unless otherwise stated, GAP Version 10, which uses the algorithm of Needleman and Wunsch (1970) J. Mol. Biol. 48 (3): 443-453, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

Isolated or recombinant nucleic acid molecules comprising nucleic acid sequences encoding cystathionine gamma-synthase or alpha′, alpha, and beta subunits of the 7S storage polypeptides or biologically active portions thereof, as well as nucleic acid molecules sufficient for use as hybridization probes to identify nucleic acid molecules encoding proteins with regions of sequence homology are provided. As used herein, the term “nucleic acid molecule” refers to DNA molecules (e.g., recombinant DNA, cDNA, genomic DNA, plastid DNA, mitochondrial DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences that encode cystathionine gamma-synthase or alpha′, alpha, and beta subunits of the 7S storage polypeptides, variants and truncations, may be synthesized and cloned into standard plasmid vectors by conventional means, or may be obtained by standard molecular biology manipulation of other constructs containing the nucleotide sequences.

In some embodiments, plants, plant parts, plant cells, seeds and methods of making and using thereof include a genome modified to contain an in-frame or frame disruption deletion. Plants, seeds, plant parts and plant cell comprising this deletion and methods of making such plants, seeds, plant parts and plant cells are provided.

In some embodiments, the nucleic acid molecule is a polynucleotide having the sequence set forth herein and variants, fragments and complements thereof. Nucleic acid sequences that are complementary to a nucleic acid sequence of the embodiments or that hybridize to a sequence of the embodiments are also encompassed. The nucleic acid sequences can be used in DNA constructs or expression cassettes for transformation and expression in organisms, including microorganisms and plants. The nucleotide or amino acid sequences may be synthetic sequences that have been designed for expression in an organism including, but not limited to, a microorganism or a plant.

In some embodiments, the nucleic acid molecule encoding the polypeptide is a non-genomic nucleic acid sequence.

In some embodiments, the nucleic acid molecule encoding a polypeptide is a non-genomic polynucleotide having a nucleotide sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity, to the nucleic acid sequence herein, wherein the encoded polypeptide is functional to increase protein or total methionine in a seed.

In some embodiments, the polynucleotide encodes a polypeptide having, or the polypeptide has, at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 3, 6, 9, 12, 15, 18, 21, 24, 27, or 30 and optionally has at least one amino acid substitution, deletion, insertion or combination therefore, compared to the native sequence.

In some embodiments, the nucleic acid molecule encodes a polypeptide comprising, or the polypeptide comprises, an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of an amino acid sequence disclosed herein.

In some embodiments, the nucleic acid encodes a polypeptide having or the polypeptide has, at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to a sequence disclosed herein. In some embodiments, the sequence identity is calculated using ClustalW algorithm in the ALIGNX® module of the Vector NTI® Program Suite (Invitrogen Corporation, Carlsbad, Calif.) with all default parameters. In some embodiments, the sequence identity is across the entire length of polypeptide calculated using ClustalW algorithm in the ALIGNX module of the Vector NTI Program Suite (Invitrogen Corporation, Carlsbad, Calif.) with all default parameters.

The embodiments also encompass nucleic acid molecules encoding cystathionine gamma-synthase or 7S storage polypeptide variants. “Variants” of the polypeptide encoding nucleic acid sequences include those sequences that encode the polypeptides disclosed herein but that differ conservatively because of the degeneracy of the genetic code as well as those that are sufficiently identical as discussed above. Naturally occurring allelic variants can be identified with the use of well-known molecular biology techniques, such as polymerase chain reaction (PCR) and hybridization techniques as outlined below. Variant nucleic acid sequences also include synthetically derived nucleic acid sequences that have been generated, for example, by using site-directed mutagenesis but which still encode the polypeptides disclosed as discussed below.

Oligonucleotide probes and methods for detecting the polynucleotides described herein are provided. Oligonucleotide probes are detectable nucleotide sequences, such as by an appropriate radioactive label or may be fluorescence as described in, for example, U.S. Pat. No. 6,268,132. As is well known in the art, if the probe molecule and nucleic acid sample hybridize by forming strong base-pairing bonds between the two molecules, it can be reasonably assumed that the probe and sample have substantial sequence homology. Preferably, hybridization is conducted under stringent conditions by techniques well-known in the art, as described, for example, in Keller and Manak (1993). Detection of the probe provides a means for determining in a known manner whether hybridization has occurred. Such a probe analysis provides a rapid method for identifying modified genes of cystathionine gamma-synthase or alpha′, alpha, and beta subunits of the 7S storage polypeptides, which modified genes and methods are provided. The nucleotide segments which are used as probes can be synthesized using a DNA synthesizer and standard procedures. These nucleotide sequences can also be used as PCR primers to amplify genes.

As is well known to those skilled in molecular biology, similarity of two nucleic acids can be characterized by their tendency to hybridize. Provided are nucleic acids that hybridize to those sequences disclosed herein under stringent conditions. As used herein the terms “stringent conditions” or “stringent hybridization conditions” are intended to refer to conditions under which a probe or nucleic acid will hybridize (anneal) to a particular sequence to a detectably greater degree than to other sequences (e.g. at least 2-fold over background).

Provided are nucleotide constructs comprising sequences described herein. The use of the term “nucleotide constructs” herein is not intended to limit the embodiments to nucleotide constructs comprising DNA. Nucleotide constructs particularly polynucleotides and oligonucleotides composed of ribonucleotides and combinations of ribonucleotides and deoxyribonucleotides may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

Provided are plants, plant cells, plant seeds and plant nuclei that are modified by gene editing. In some embodiments, gene editing may be facilitated through the induction of a double-stranded break (DSB) or single-strand break, in a defined position in the genome near the desired alteration. DSBs can be induced using any DSB-inducing agent available, including, but not limited to, TALENs (transcription activator-like effector nucleases), meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template. In some embodiments, the methods do not use TALENs enzymes or technology and plants and seeds are produced from methods which do not use TALENs enzymes or technology.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10:957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site.

The term “variable targeting domain” or “VT domain” is used interchangeably herein and includes a nucleotide sequence that can hybridize (is complementary) to one strand (nucleotide sequence) of a double strand DNA target site. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that can transcribe the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41:4336-4343; Ma et al., Mol. Ther. Nucleic Acids 3: e161) as described in WO2016025131, published on Feb. 18, 2016, incorporated herein in its entirety by reference.

Provided are plants, plant cells, plant seeds and plant nuclei that are transformed with sequences described herein. Transformation may be stable or transient. “Stable transformation” as used herein means that the nucleotide construct introduced into a plant integrates into the genome of the plant and is capable of being inherited by the progeny thereof. “Transient transformation” as used herein means that a polynucleotide is introduced into the plant and does not integrate into the genome of the plant or a polypeptide is introduced into a plant. “Plant” as used herein refers to whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells and pollen).

Transformation methods include introduction of a recombinant DNA construct comprising an expression cassette. Provided are constructs which include one or more heterologous promoter sequences operably connected to one or more polynucleotides encoding polypeptides disclosed herein and appropriate transcription termination sequences and plants, seeds, cells and nuclei containing the recombinant DNA construct or expression cassette.

Transformation methods include introduction of a suppression DNA construct or a construct that results in increased expression of a target gene, such as encoding the cystathionine gamma-synthase or alpha′, alpha, and beta subunits of the 7S storage polypeptides. “Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The term “suppression” includes lower, reduce, decline, decrease, inhibit, eliminate and prevent. “Silencing” or “gene silencing” does not specify mechanism and is inclusive, and not limited to, anti-sense, cosuppression, viral-suppression, hairpin suppression, stem-loop suppression, RNAi-based approaches and small RNA-based approaches.

The embodiments further relate to plant-propagating material of a transformed plant of the embodiments including, but not limited to, seeds, tubers, corms, bulbs, leaves and cuttings of roots and shoots. Methods of plant breeding by crossing a modified plant described herein with a second different plant are provided. Progeny plants, plant cells, seeds and plant nuclei from such breeding methods are provided, such as F1 progeny plants, plant cells, seeds and plant nuclei.

Transformation of any plant species can be carried out, including, but not limited to, monocots and dicots. Examples of plants of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), Citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oat (Avena sativa), barley (Hordeum vulgare), vegetables, ornamentals, and conifers.

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive, etc. Leguminous plants include beans and peas. Beans include guar, locust bean, fenugreek, soybean, garden beans, cowpea, mung bean, lima bean, fava bean, lentils, chickpea, etc.

The methods comprise providing a plant or plant cell expressing a polynucleotide encoding the polypeptide sequence disclosed herein and growing the plant or a seed thereof in a field. In some embodiments, the expression of the modified polypeptide results in a plant producing increased yield or biomass.

As defined herein, the “yield” of the plant refers to the quality and/or quantity of biomass produced by the plant. “Biomass” as used herein refers to any measured plant product. An increase in biomass production is any improvement in the yield of the measured plant product. Increasing plant yield has several commercial applications. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not expressing the modified sequence.

Methods of detecting the modified polynucleotides are provided. Methods of extracting modified DNA from a sample or detecting the presence of DNA corresponding to the modified genomic sequences comprising deletions, can be carried out. Sequences may contain a feature following which feature can be any can be A, G, C or T. For example, an “n” at a position can be entirely absent or a length of 0 to 1, 0 to 2, 0 to 3, 0 to 4, 0 to 5, 0 to 10, 0 to 15, 0 to 20, 0 to 25, 0 to 30, 0 to 35, 0 to 40, or 0 to 45 nucleotides, and when present may contain any combination of C, T, G or A. Such methods of detecting polynucleotides comprise contacting a sample comprising soybean genomic DNA with a DNA primer set, that when used in a nucleic acid amplification reaction, such as the polymerase chain reaction (PCR), with genomic DNA extracted from soybeans produces an amplicon that is diagnostic for either the presence or absence of the deleted sequence, cystathionine gamma-synthase coding sequence or alpha′, alpha, and beta subunits of the 7S storage protein coding sequences. The methods include the steps of performing a nucleic acid amplification reaction, thereby producing the amplicon and detecting the amplicon.

In some embodiments one of the pair of DNA molecules comprises the wild type sequence where the modification occurs with the second of the pair being upstream or downstream as appropriate and suitably in proximity to the wild type sequence where the modification occurs, such that an amplicon is produced when the wild type sequence is present, but no amplicon is produced when the deletion is present. In the context of the methods, in proximity means sufficiently close such that the distance between the first and second of the pair of DNA molecules facilitates the production of an amplicon when included in a DNA amplification reaction comprising soybean genomic DNA. For example, the second primer may bind at a location beginning at, within or less than 1, 2, 5, 10, 20, 30, 40, 50, 60, 70, 80, 90, 100, 150, 200, 250, 300, 350, 400, 450, 500, 600, 700, 800, 900, 1000, 1500, 16, 1700, 1800, 1900, 2000, 2100, 2200, 2300, 2400, 2500, 2600, 2700, 2800, 2900, 3000, 3500, 4000, 4500, 5000, 5500, 6000, 6500, 7000, 7500, 8000, 8500, 9000, 9500 or 10000 nucleotides upstream or downstream of the end of the binding site of the first DNA primer molecule.

Probes and primers are provided which are of sufficient nucleotide length to bind specifically to the target DNA sequence under the reaction or hybridization conditions. Suitable probes and primers are at least 10, 11, 12, 13, 14, 15, 16, 17, 18, 19, 20, 21, 22, 23, 24, 25, 26, 27, 28, 29 or 30 nucleotides in length, and less than 35, 34, 33, 32, 31, 30, 29, 28, 27, 26, 2, 5 2, 4 23, 22, 21, 20, 19, 18, 17, 16, 15, 14, 13, or 12 nucleotides in length. Such probes and primers can hybridize specifically to a target sequence under high stringency hybridization conditions. Preferably, probes and primers have complete or 100% DNA sequence similarity of contiguous nucleotides with the target sequence, although probes which differ from the target DNA sequence but retain the ability to hybridize to target DNA sequence may also be used. Reverse complements of the primers and probes disclosed herein are also provided and can be used in the methods and compositions described herein.

In some embodiments, one of the pair of DNA molecules comprises the modification or traverses the modification junction, such as the deletion junction, with the second DNA molecule of the pair being upstream or downstream of the genomic sequence as appropriate, such that an amplicon is produced when the modified allele is present, but no amplicon is produced when the wild type allele is present. Suitable primers for use in reactions to detect the presence of the modified alleles can be designed based on the junction sequences described herein. In some embodiments, the first or second primer molecule binds to a sequence corresponding to the position, or a complement thereof. In some embodiments, the primers bind to the target sequence to produce an amplicon of a length described herein. The amplicon molecule produced can be at least 5, 10, 15, 20, 25, 30, 35, 40, 41, 42, 43, 44, 45, 46, 47, 48, 49, 50, 60, 70, 80, 90, 100, 125, 150, 175, 200, 225, 250, 275, 300, 350, 400, 450, 500, 550, 600, 650, 700, 750, 800, 850, 900, 950, 1000, 1500 or 2000 nucleotides in length and less than about 10000, 9000, 8000, 7500, 7000, 6500, 6000, 5500, 5000, 4500, 4000, 3500, 3000, 2500, 2000, or 1500 nucleotides in length.

Gene-specific primers for use in genotyping soybean cystathionine gamma-synthases and 7S storage proteins are provided in Table 2.

TABLE 2

| | |
|---|---|
| F-Glyma.20G146200-geno | ACCTGGCAGATATGATGTATGCAC |
| | (SEQ ID NO: 115) |
| R-Glyma.20G146200-geno | GACAGCCAGCCACATCAGCCATG |
| | (SEQ ID NO: 116) |
| F-Glyma.20G148200-geno | ACCTGGCAGATATGATGTATGCAC |
| | (SEQ ID NO: 117) |
| R-Glyma.20G148200-geno | GAACTATGTATCATGATCACACC |
| | (SEQ ID NO: 118) |
| F-Glyma.20G148400-geno | TGATGCCCTGAGAGTCCCCTCAG |
| | (SEQ ID NO: 119) |
| R-Glyma.20G148400-geno | CTTCAGAGTAAATTACTTCTGCGT |
| | (SEQ ID NO: 120) |
| F-Glyma.10G246500-geno | CTACCGTGTTGTAGAGCTCATGGC |
| | (SEQ ID NO: 121) |
| R-Glyma.10G246500-geno | ACCCAATCAGCATGCTTCCACATG |
| | (SEQ ID NO: 122) |
| F-Glyma.10G028300-geno | CGTGATCTGGGACTGATCTTGAC |
| | (SEQ ID NO: 123) |
| R-Glyma.10G028300-geno | ACATTCCCCGTGATCTTCAGCC |
| | (SEQ ID NO: 124 |
| F-Glyma.02G145700-geno | CTTATTGTATGGATGGTATAGTC |
| | (SEQ ID NO: 125) |
| R-Glyma.02G145700-geno | TGTGTACTCCTACCTGCAAACG |
| | (SEQ ID NO: 126) |
| F-Glyma.20G148300-geno | ATCCAACTGTCACACTACACTGGC |
| | (SEQ ID NO: 127) |
| R-Glyma.20G148300-geno | AAGCATCATATGCTGTCCTGTCG |
| | (SEQ ID NO: 128) |
| F-Glyma.10G246300-geno | GCGATGCCCTAAGAGTCCCTGCAG |
| | (SEQ ID NO: 129) |
| R-Glyma.10G246300-geno | TAGCTCAGTCAGCTATGTTTCAC |
| | (SEQ ID NO: 130) |
| F-GmCGS1-geno | GACAAAGGAGTGACGCGTCACG |
| | (SEQ ID NO: 131) |
| R-GmCGS1-geno | CAACATATGAAGCAGTTTCCATGC |
| | (SEQ ID NO: 132) |
| F-GmCGS2-geno | ACCCCATCGCAAATCGAAGAGTAC |
| | (SEQ ID NO: 133) |
| R-GmCGS2-geno | GACGTCCTCGGCGGCGGCGAGGT |
| | (SEQ ID NO: 134) |

Unless expressly stated or apparent from the context of usage, the methods and compositions of the present disclosure can be used with any plant species including, for example, monocotyledonous plants and dicotyledonous plants. Examples of plant species of interest include, but are not limited to, corn (Zea mays), Brassica sp. (e.g., B. napus, B. rapa, B. juncea), alfalfa (Medicago sativa), rice (Oryza sativa), rye (Secale cereale), triticale (xTriticosecale or Triticum×Secale) sorghum (Sorghum bicolor, Sorghum vulgare), teff (Eragrostis tef), millet (e.g. pearl millet (Pennisetum glaucum), proso millet (Panicum miliaceum), foxtail millet (Setaria italica), finger millet (Eleusine coracana), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanut (Arachis hypogaea), cotton (Gossypium barbadense, Gossvpium hirsutum), strawberry (e.g. Fragariaxananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachwya, D. bulbifera, D. esculenta, D. dumetorum, D. trifida), cassava (Manihot esculenta), coffee (Coffea spp.), coconut (Cocos nucifera), okra (Abelmoschus esculentus), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), grape (Vitis vinifera), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon, tomato (Solanum lycopersicum), eggplant (Solanum melongena), pepper (Capsicum annuum), lettuce (e.g. Lactuca sativa), bean (Phaseolus vulgaris), lima bean (Phaseolus limensis), pea (Lathyrus spp.), chickpea (Cicer arietinum), and members of the genus Cucumis such as cucumber (C. sativus), cantaloupe (C. cantalupensis), and musk melon (C. melo), and ornamentals. Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and Chrysanthemum. In certain embodiments, the plant is a Brassica, wheat, maize, potato, soybean, or cotton plant.

Also provided are progeny plants and seeds thereof comprising a polynucleotide or modification of the present disclosure. The present disclosure also provides fruits, seeds, tubers, leaves, stems, roots, and other plant parts produced by the plants and/or progeny plants of the disclosure as well as biological samples comprising, or produced or derived from, the plants or any part or parts thereof including, but not limited to, fruits, tubers, leaves, stems, roots, and seed. In certain embodiments, the biological sample is a commodity plant product. As used herein, a “commodity plant product” refers to any composition or product that is comprised of material derived from a plant, seed, plant cell, or plant part of the present disclosure. Commodity plant products may be sold to consumers and can be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, paper, tea, coffee, silage, crushed of whole grain, and any other food for human or animal consumption; and biomasses and fuel products; and raw material in industry. Industrial uses of oils derived from the agricultural plants described herein include ingredients for paints, plastics, fibers, detergents, cosmetics, lubricants, and biodiesel fuel. Commodity plant products also include industrial compounds, such as a wide variety of resins used in the formulation of adhesives, films, plastics, paints, coatings and foams. It is recognized that such commodity plant products can be consumed or used by humans and other animals including, but not limited to, pets (e.g. dogs and cats), livestock (e.g. pigs, cows, chickens, turkeys, and ducks), and animals produced in freshwater and marine aquaculture systems (e.g. fish, shrimp, prawns, crayfish, and lobsters).

### EMBODIMENTS

The following numbered embodiments also form part of the present disclosure:
1. A plant, plant part, or seed comprising one or more modifications that increase seed protein content, the modification selected from a deletion, insertion, or substitution of a nucleotide in (i) a genomic sequence encoding a cystathionine gamma-synthase (CGS), the modification resulting in suppression of the feedback regulation of the same while retaining enzymatic activity, and/or (ii) a genomic sequence encoding a low methionine content seed storage protein, the modification resulting in suppression of the activity of the same.
2. The plant, plant part, or seed of embodiment 1, wherein the modification comprises a deletion, insertion, or substitution of a nucleotide at or near the MTO1 region of the genomic sequence encoding the CGS.
3. The plant, plant part, or seed of embodiment 1 or embodiment 2, wherein the modification is an in-frame modification of the genomic sequence encoding the CGS.
4. The plant, plant part, or seed of any one of embodiments 1-3, wherein the deletion or insertion results in a frameshift mutation of the genomic sequence encoding the low methionine content seed storage protein.
5. The plant, plant part, or seed of any one of embodiments 1-4, wherein the plant, plant part, or seed comprises a modification selected from a deletion, insertion, or substitution of a nucleotide in a genomic sequence encoding a beta-conglycinin storage protein.
6. The plant, plant part, or seed of any one of embodiments 1-5, wherein the plant, plant part, or seed comprises a modification selected from a deletion, insertion, or substitution of a nucleotide in a genomic sequence encoding an alpha′, alpha, and beta subunit of the 7S storage protein.
7. The plant, plant part, or seed of any one of embodiments 1-6, further comprising a heterologous nucleic acid sequence selected from a reporter gene, a selection marker, a disease resistance gene, a herbicide resistance gene, an insect resistance gene, a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance, and a gene involved in salt resistance in plants.
8. The plant, plant part, or seed of any one of embodiments 1-7, wherein protein content of the seed is increased relative to a control seed not comprising the modification.
9. The plant, plant part, or seed of any one of embodiments 1-8, wherein methionine content of the seed is increased relative to a control seed not comprising the modification.
10. The plant, plant part, or seed of any one of embodiments 1-9, wherein proteome rebalancing results in an increase in high methionine seed storage proteins in the seed.
11. The plant, plant part, or seed of any one of embodiments 1-10, wherein 11S glycinin polypeptide content of the seed is increased relative to a control seed not comprising the modification as a result of proteome rebalancing.
12. The plant, plant part, or seed of any one of embodiments 1-11, wherein the modification is homozygous.
13. The plant, plant part, or seed of any one of embodiments 1-12, wherein the genomic sequence encoding the CGS comprises a nucleotide sequence having at least 80%, at least 90%, at least 95%, at least 98%, at least 99%, or 100% sequence identity to SEQ ID NO: 1 or 4.
14. The plant, plant part, or seed of any one of embodiments 1-13, wherein the genomic sequence encoding the low methionine content seed storage protein comprises a nucleotide sequence having at least 80%, at least 90%, at least 95%, at least 98%, at least 99%, or 100% sequence identity to SEQ ID NO: 7, 10, 13, 16, 19, 22, 25, or 28.
15. A method of making a modified plant with high seed protein content compared to the seed protein content of a nonmodified plant, the method comprising: (a) crossing a first plant comprising a deletion, insertion, or substitution of a nucleotide in (i) a genomic sequence encoding cystathionine gamma-synthase (CGS), the modification resulting in suppression of the feedback regulation of the same while retaining enzymatic activity, and/or (ii) a genomic sequence encoding a low methionine content seed storage protein, the modification resulting in suppression of the activity of the same, with a second plant; and (b) selecting a progeny plant, or plant part thereof, with high protein content for further breeding.
16. The method of embodiment 15, wherein the modified plant is an F1 progeny plant.
17. A method of producing high protein seed meal comprising: harvesting the seed of any one of embodiments 1-14 from a plant comprising the modification and processing the seed to form meal.
18. A modified CGS polynucleotide comprising an in-frame insertion or deletion in the MTO1 region so that feedback inhibition of expression is suppressed.
19. The modified CGS polynucleotide of embodiment 18, wherein the polynucleotide comprises the sequence of SEQ ID NO: 44, 45, 46, 48, 49, 50, or 51.
20. The modified CGS polynucleotide of embodiment 18 or embodiment 19, wherein the polynucleotide comprises a sequence having at least 80%, at least 90%, at least 95%, at least 98%, at least 99%, or 100% sequence identity to SEQ ID NO: 52, 53, 54, 56, 58, 60, or 61.
21. A modified 7S polynucleotide comprising an insertion, deletion, or substitution that suppresses activity of the polynucleotide.
22. The modified 7S polynucleotide of embodiment 21, wherein the polynucleotide comprises a sequence having at least 80%, at least 90%, at least 95%, at least 98%, at least 99%, or 100% sequence identity to one or more of SEQ ID NOs: 62-108.
23. A plant cell comprising the polynucleotide of any one of embodiments 18-22.
24. The plant cell of embodiment 23, wherein the plant cell is a soybean plant cell.
25. A plant comprising the plant cell of embodiment 23 or embodiment 24.
26. A seed of the plant of embodiment 25, wherein the seed has increased protein content compared to a seed comprising a comparable polynucleotide without the modification.
27. A seed of the plant of embodiment 25, wherein the seed has increased methionine content compared to a seed comprising a comparable polynucleotide without the modification.
28. A modified polypeptide encoded by the polynucleotide of any one of embodiments 18-22.
29. A method of producing a plant cell having increased protein content, the method comprising: introducing a double-strand-break-inducing (DSB-inducing) agent to a plant cell, wherein the DSB-inducing agent creates a double-strand break in an endogenous CGS gene and/or a low methionine content seed storage protein gene in the genome of the plant, wherein the repair of the DSB results in the production of a modified polynucleotide, and wherein the production of the modified polynucleotide produces increased protein content in the seed.
30. The method of embodiment 29, wherein the modified polynucleotide includes a deletion, insertion, or substitution in the MTO1 region of the CGS gene.
31. The method of embodiment 30, wherein the deletion or insertion is an in-frame deletion or insertion.
32. The method of any one of embodiments 29-31, wherein the modified polynucleotide is a deletion, insertion, or substitution of a 7S beta-conglycinin.
33. The method of any one of embodiments 29-32, wherein the endogenous CGS gene comprises a nucleotide sequence having at least 80%, at least 90%, at least 95%, at least 98%, at least 99%, or 100% sequence identity to SEQ ID NO: 1 or 4.
34. The method of any one of embodiments 29-33, wherein the endogenous low methionine content seed storage protein gene comprises a nucleotide sequence having at least 80%, at least 90%, at least 95%, at least 98%, at least 99%, or 100% sequence identity to SEQ ID NO: 7, 10, 13, 16, 19, 22, 25, or 28.
35. A method of editing the genome of a plant cell to produce a plant cell having increased protein, the method comprising: introducing a guide RNA, a polynucleotide modification template, and a Cas endonuclease to a plant cell; and regenerating a plant from the plant cell wherein the genome of the regenerated plant comprises the modified polynucleotide of any one of embodiments 18-22.
36. The method of embodiment 35, wherein the seed of the plant has increased protein content compared with a comparable seed not comprising the modified polynucleotide.
37. The method of embodiment 35 or embodiment 36, wherein the seed of the plant has increased methionine content compared with a comparable seed not comprising the modified polynucleotide.
38. The method of any one of embodiments 35-37, wherein the genome is modified by a site-directed endonuclease.
39. The method of any one of embodiments 35-38, wherein the plant cell is a soybean cell.
40. A method of producing a plant cell having increased seed protein content, the method comprising: transforming a plant cell with the modified polynucleotide of any one of embodiments 18-22, wherein a plant regenerated from the transformed plant cell has increased seed protein content compared with a comparable plant cell not comprising the modified polynucleotide.
41. A polynucleotide construct comprising a sequence encoding a guide RNA which targets an endogenous CGS gene and/or a low methionine content storage polypeptide gene when expressed in a plant cell and thereby generates a modified endogenous CGS gene and/or a low methionine content storage polypeptide gene.
42. The polynucleotide construct of embodiment 41, wherein the guide RNA comprises the spacer sequence of SEQ ID NO: 109, 110, 111, 112, 113, or 114.
43. A plant cell comprising the polynucleotide construct of embodiment 41.
44. The plant cell of embodiment 43, wherein the plant cell is a soybean cell.
45. A method of producing a soybean seed comprising: a) sexually crossing a first soybean line comprising the polynucleotide of any one of embodiments 18-22 with a second soybean line not comprising the polynucleotide; and b) harvesting the seed produced thereby.
46. The method of embodiment 45 further comprising the step of backcrossing a second generation progeny plant that comprises the polynucleotide to the parent plant that lacks the polynucleotide, thereby producing a backcross progeny plant that produces seed with increased protein content.
47. A method of screening for the presence or absence of the polynucleotide of any one of embodiments 18-22 in a plurality of genomic soybean DNA samples, the method comprising the steps of (a) contacting a plurality of genomic soybean DNA samples, at least some of which comprise the polynucleotides, with a first DNA primer molecule and a second DNA primer molecule; (b) providing a plurality nucleic acid amplification reaction conditions; (c) performing the nucleic acid amplification reactions, thereby producing a DNA amplicon molecule indicating the presence of the polynucleotide or a wild-type CGS and/or a low methionine content storage polypeptide gene nucleotide sequence; and (d) detecting the DNA amplicon molecules, wherein the presence, absence or size of the DNA amplicon molecule indicates the presence or absence of the polynucleotide in the at least one of the plurality of genomic soybean DNA samples.
48. The method of embodiment 47, wherein the first DNA primer molecule or the second DNA primer molecule comprises the sequence of one or more of SEQ ID NOs: 115-134.
49. An animal feed comprising the plant, plant part, or seed of any one of embodiments 1-14.

The foregoing invention has been described in detail by way of illustration and example for purposes of clarity and understanding. As is readily apparent to one skilled in the art, the foregoing disclosures are only some of the methods and compositions that illustrate the embodiments of the foregoing invention. It will be apparent to those of ordinary skill in the art that variations, changes, modifications, and alterations may be applied to the compositions and/or methods described herein without departing from the true spirit, concept, and scope of the invention.

All publications, patents, and patent applications mentioned in the specification are incorporated by reference herein for the purpose cited to the same extent as if each was specifically and individually indicated to be incorporated by reference herein.

The following examples illustrate particular aspects of the disclosure and are not intended in any way to limit the disclosure.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1. In-Frame Mutations in Arabidopsis CGS1 Using CRIPSR/Cas9 to Enhance Free Met Pool

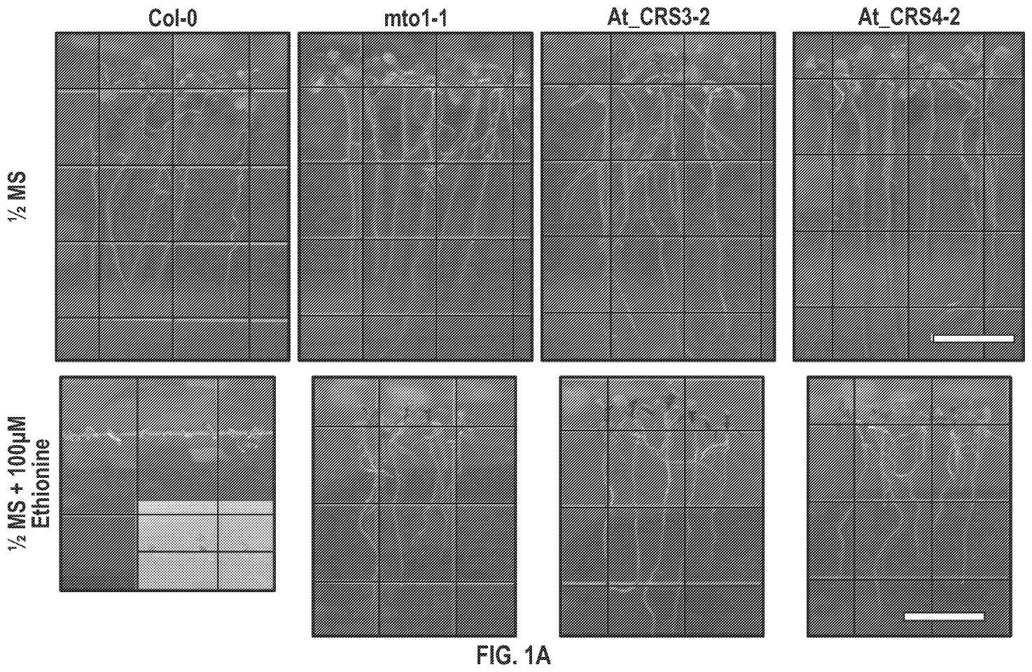

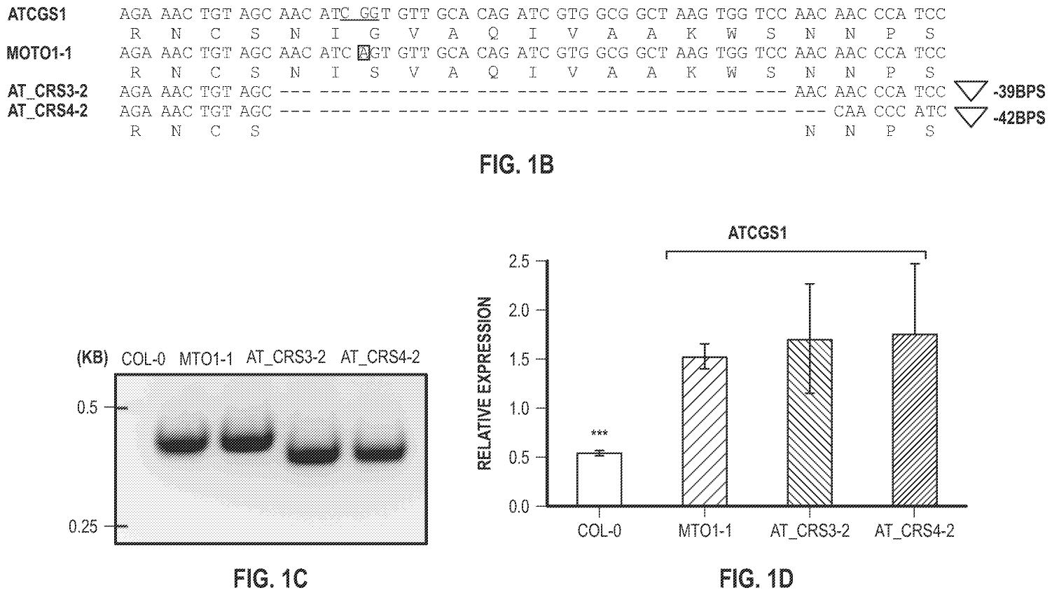

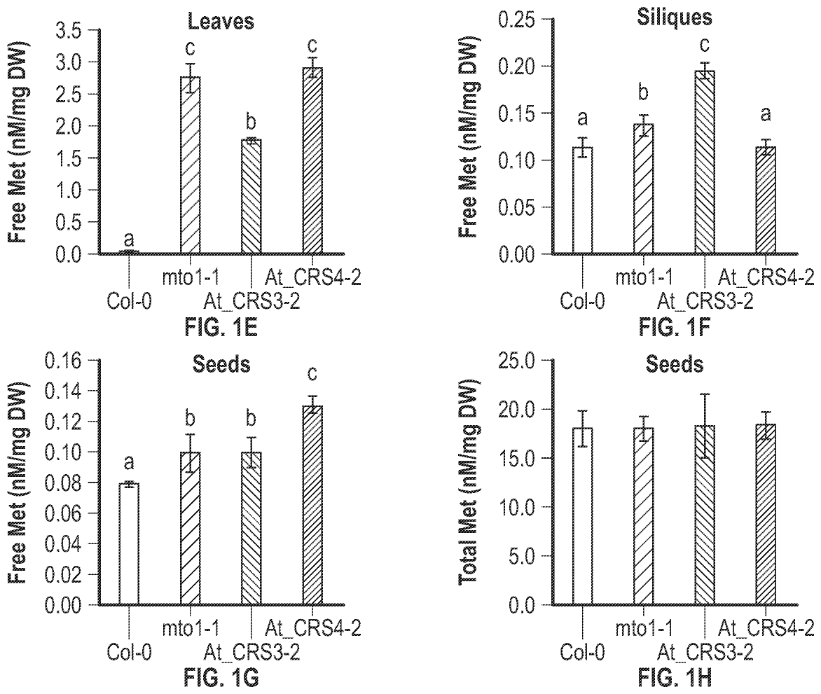

In Arabidopsis, the AtCGS1 gene encodes protein which catalyzes the first committed step in Met biosynthesis, and its expression is regulated by a negative feedback-inhibition in response to S-adenosyl-L-methionine (AdoMet), a direct metabolite of methionine. Several single-base mutations within this MTO1 region relieve feedback inhibition, leading to over-accumulation of free Methionine content in vegetative tissues. Therefore, it was hypothesized that in-frame mutations within the MTO1 region could also create feedback-insensitive CGS, a phenotypic mimic of EMS mutants, and increase free Met content. A CRISPR/Cas9 vector designed to create mutations within the MTO1 was constructed. More than a hundred T1 Arabidopsis transgenic lines were screened on plates containing half-strength MS supplemented with 100 μM DL-ethionine, a toxic analogue of Met, to identify the putative over-accumulate Met thionine CRISPR lines. Two independent lines (At_CRS3-2 and At_CRS4-2) showing normal growth were identified (FIG. 1A). The phenotypes of two CRISPR mutant lines were similar to mto1-1, an AtCGS1 EMS mutant line. In contrast, the growth of wild-type Col-0 was completely inhibited in the presence of ethionine (FIG. 1A). PCR and Sanger sequencing of PCR amplicons of the AtCGS1 gene from two CRISPR mutant lines showed that At_CRS3-2 and At_CRS4-2 harbored CRISPR/Cas9-induced in-frame deletions of 39 and 42 base pairs, respectively, within and extended MTO1 region (FIG. 1B-C). The transcript levels of AtCGS1 from leaves of two weeks post germination on the ½ MS media of the CRISPR lines were quantified and compared with wildtype Col-0 and mto1-1 genotypes. While the mRNA level of AtCGS1 of the CRISPR lines were no difference to the mto1-1, they were 3-fold higher than wild-type plants under the ½ MS media (FIG. 1D). Free amino acid analysis was performed 3-week-old leaves siliques and mature seeds; whereas, total amino acid contents were quantified on mature seeds of wild-type, mto1-1 and two in-frame CRISPR mutants. Free Met content in the leaves of the EMS, mto1-1 mutant and the in-frame CRISPR mutants were 38 to 60 fold higher than the wild-type. A significant increase in Free Met was found in siliques and mature seeds of mutant lines (FIG. 1E-G). Although Free Met content increased in leaves, siliques and mature seeds, soluble amino acids that are metabolically related to Methionine did not significantly differ between the wild-type and mutant lines. Total amino acids were measured in mature seed; however, there was no difference in total Met content and other amino acids among genotypes (FIG. 1H). Taken together, the data showed that induced in-frame mutations within the MTO1 region abolished the negative feedback inhibition on AtCGS1 expression and led to over-accumulated free Met in leaves and seeds. However, mutation in MTO1 region alone did not improve the total Met content in Arabidopsis seeds.

### Example 2. Multiplex CRISPR/Cas9 Based Genome Editing for Improving Methionine and Protein Content in Soybean Seeds

Soybean (Glycine max (L.) Merr) is one of the most important crops grown worldwide, providing 70% and 28% of protein meal and vegetable oil consumption, respectively. Soy meal contains high protein content and well-balanced amino acid profile; however, the content of sulfur containing amino acids cysteine (Cys) and methionine (Met) in soybean seed is not found at adequate levels for optimal growth and development of monogastric animals. Supplementation of synthetic amino acids is added to meet the nutritional requirements in soy-based animal feed, though it significantly increases the production cost. In this example, multiplex CRISPR/Cas9 based editing was used to generate mutant soybean lines with in-frame deletions in the MTO1 region of CGS1 and 7S knock-out to provide increased Met and protein in soybean seed. Creating soybean lines with mutations of CGS and Cupin gene family by CRISRP/Cas9

The MTO1 region is highly conserved in plants and in-frame mutation of the MTO1 region led to a significant increase in free Met in Arabidopsis (Example 1). In addition, knock down of seed storage proteins in soybean greatly changes soybean seed protein profiles. Therefore, it was hypothesized that the combination of null mutation of seed storage proteins (Cupin gene family) and an in-frame mutant of soybean CGS would increase the free Met and total Met in soybean seed.

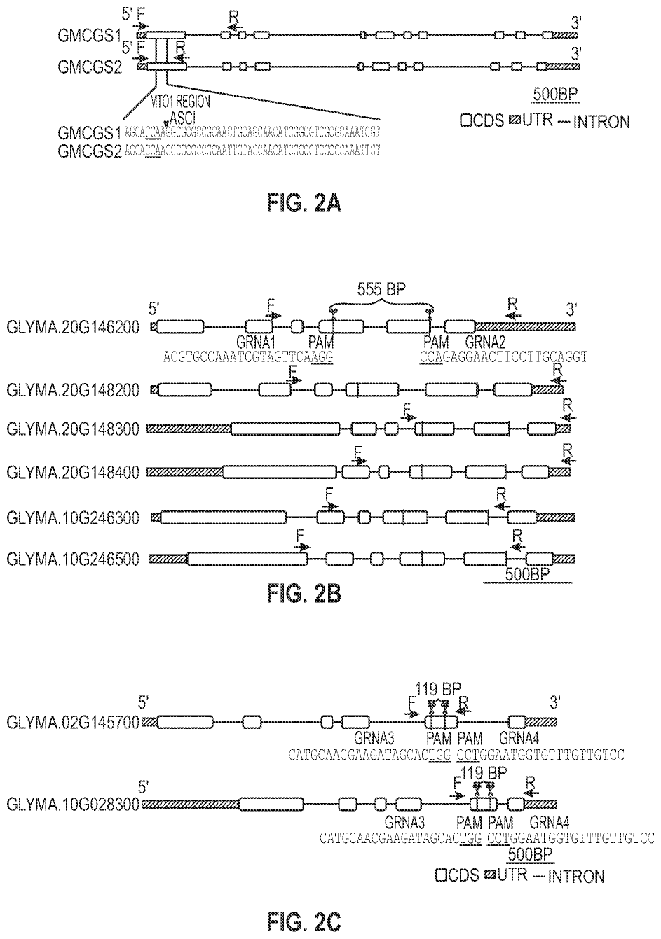

A CRISPR/Cas9 construct with five single guide RNAs (sgRNAs) to target the MTO1 region of two soybean CGS genes [GmCGS1 (Glyma.09g235400) and GmCGS2 (Glyma.18g261600)] and the conserved exon region of eight members of soybean seed specific Cupin gene family [Glyma.20G148300, Glyma.20G148400 encoding 7S α subunits; Glyma.10G246500, Glyma. 10G246300 and Glyma. 10g028300 encoding 7S α′ subunits; Glyma.20G146200, Glyma.20G148200 encoding 7S β-subunits; Glyma.02g145700 encoding SBP1 (Sucrose Binding Protein 1)] was constructed and transformed into soybean cultivar Maverick (FIG. 2A-C). Mutant lines that segregated from Cas9 transgenic lines were used in this example to avoid further gene edits.

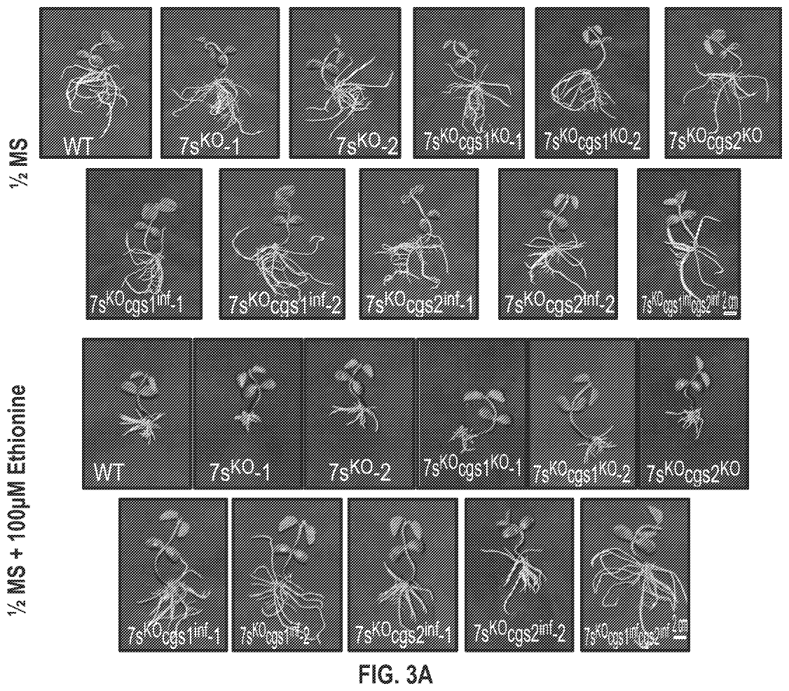

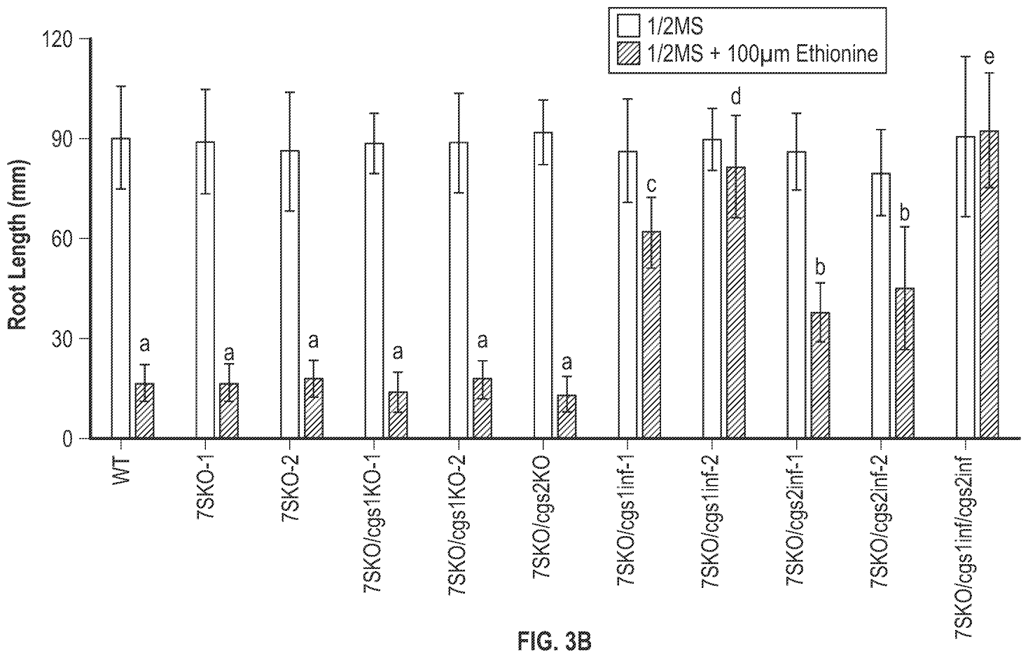

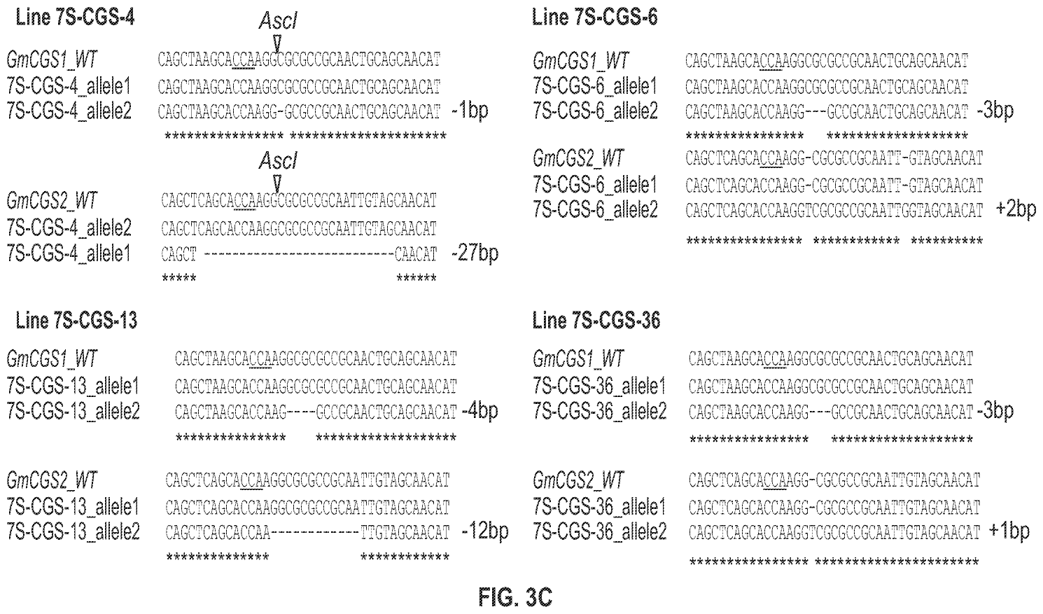

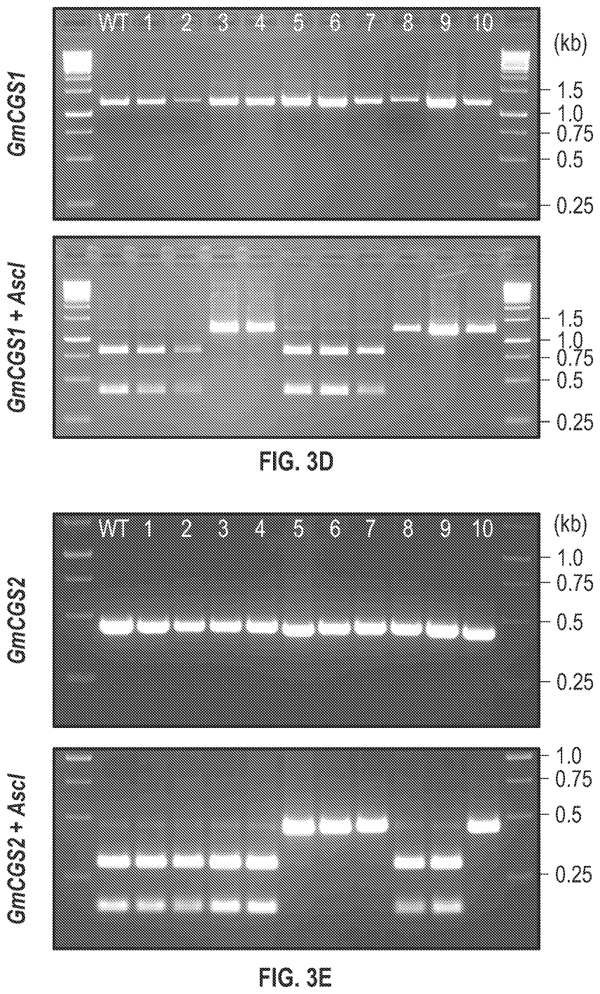

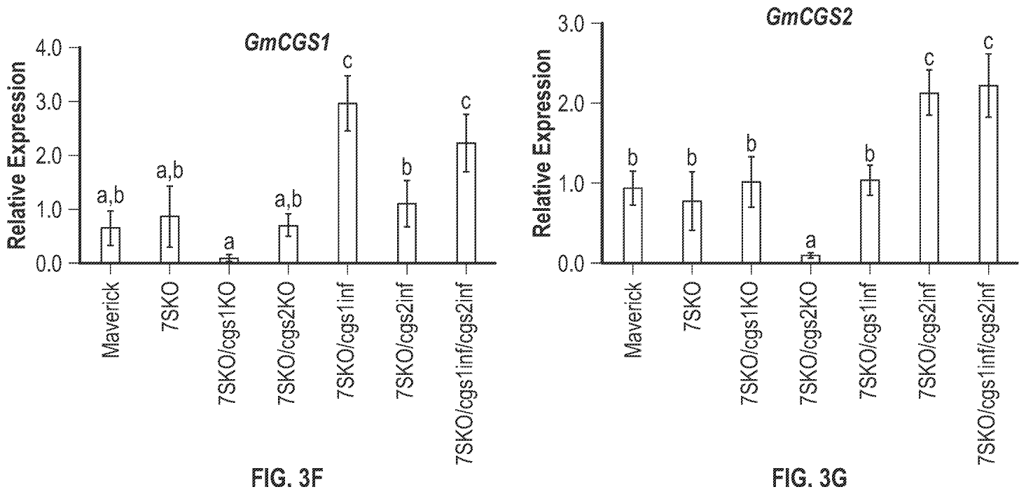

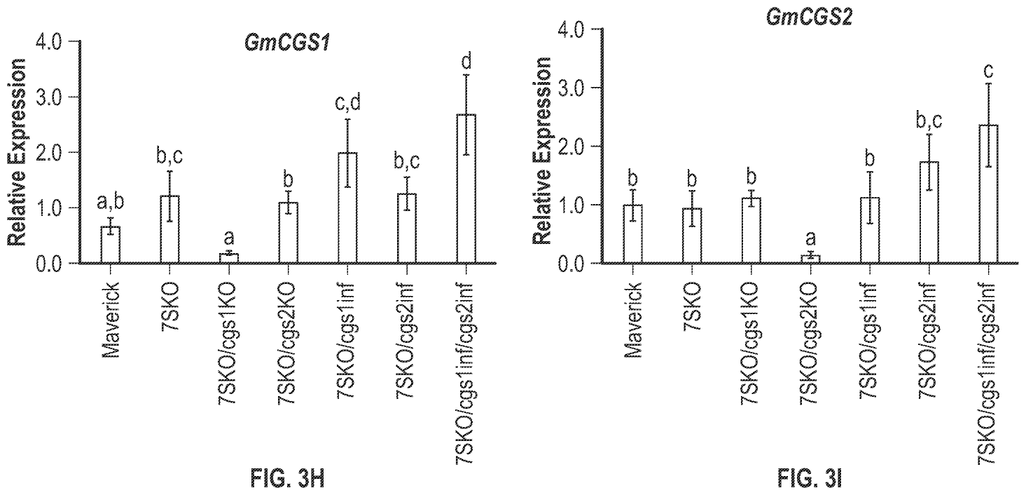

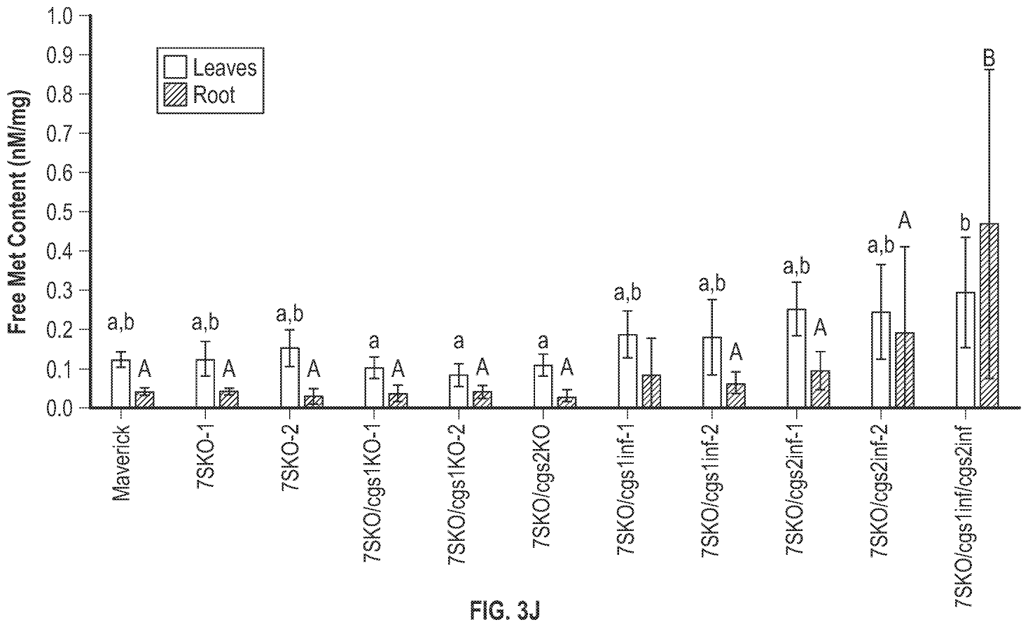

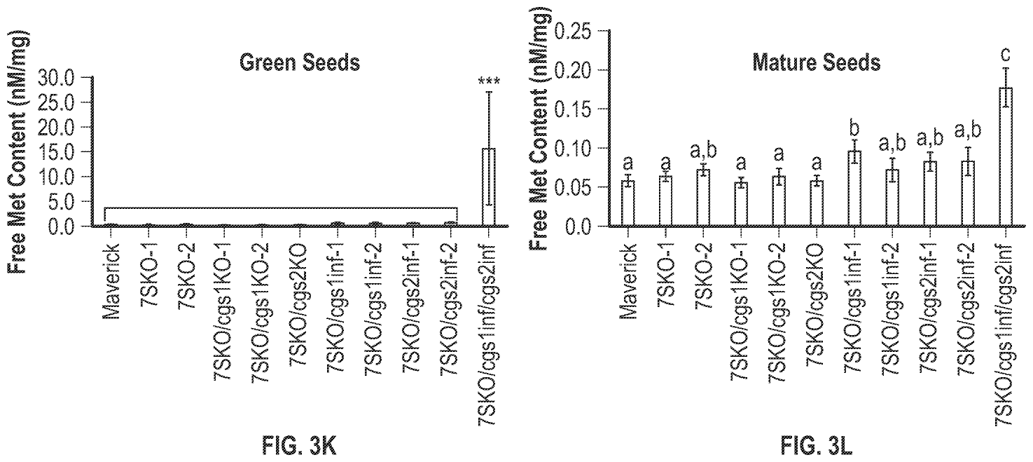

To identify feedback insensitive CGS in soybean, seeds were sown on media supplemented with ethionine. Ten days after sowing, soybean root length was 6-fold shorter than those sown in normal conditions (FIG. 3A-B). Therefore, this screening approach was used to identify mutant lines that possessed in-frame mutations in the MTO1 region of GmCGS genes. Out of 16 CRISPR lines, certain T1 seedlings from four independent lines (7S-CGS-4, -6, -13, and -36) exhibited normal growth on ½ MS media supplemented with 50 μM ethionine, and they segregated for ethionine resistant phenotype. A Cleaved Amplified Polymorphic Sequences (CAPS) marker was developed for genotyping based on the insensitivity of the DNA sequence to AscI restriction enzyme. Sanger sequencing and genotyping showed that these four T0 lines were heterozygous mutations (either frameshift (KO) or in-frame mutations) for GmCGS1 and GmCGS2: 7S-CGS-4 gmcgs1 (1-bp deletion, frameshift mutation)/gmcgs2 (27-bp deletion, in-frame mutation); 7S-CGS-6 gmcgs1 (3-bp deletion, in-frame mutation)/gmcgs2 (2-bp insertion, KO mutation); 7S-CGS-13 gmcgs1 (4-bp deletion, KO mutation)/gmcgs2 (12-bp deletion, in-frame mutation); 7S-CGS-36 gmcgs1 (3-bp deletion, in-frame mutation)/gmcgs2 (1-bp insertion, KO mutation) (FIG. 3C, Table 3).

TABLE 3

| CGS | MTO1 region | Genomic | Amino acid |
|---|---|---|---|
| modification | sequence | sequence | sequence |
| CGS1 (−1 | SEQ ID | SEQ ID | — |
| deletion) | NO: 44 | NO: 52 | |
| CGS1 (−4 | SEQ ID | SEQ ID | — |
| deletion) | NO: 45 | NO: 53 | |
| CGS1 (−3 | SEQ ID | SEQ ID | SEQ ID |
| deletion) | NO: 46 | NO: 54 | NO: 55 |
| CGS2 (−27 | SEQ ID | SEQ ID | SEQ ID |
| deletion) | NO: 48 | NO: 56 | NO: 57 |
| CGS2 (−12 | SEQ ID | SEQ ID | SEQ ID |
| deletion) | NO: 49 | NO: 58 | NO: 59 |
| CGS2 (+2 | SEQ ID | SEQ ID | — |
| insertion) | NO: 50 | NO: 60 | |
| CGS2 (+1 | SEQ ID | SEQ ID | — |
| insertion) | NO: 51 | NO: 61 | |

To identify 7S β-conglycinin mutations, the soluble protein from pooled T1 seed samples of the 16 T0 lines were analyzed with SDS-PAGE. 7S β-conglycinin and SBP1 fractions from five CRISPR lines (7S-CGS-4, 7S-CGS-6, 7S-CGS-13, 7S-CGS-15, and 7S-CGS-36) were absent on the SDS-PAGE as compared to the wild-type. The CRISPR lines 7S-CGS-4, 7S-CGS-6, 7S-CGS-13, and 7S-CGS-36 were chosen for further analysis because they were also resistant to ethionine as discussed above. Specific PCR amplicons of 7S genes which amplified DNA samples of T0 generation of the 4 CRISPR lines were sequenced. These 4 lines were homozygous and biallelic for small, large deletions or insertions as well as inversions of 7S genes (SEQ ID NOs: 62-108).

Ethionine screening, CAPS marker analysis, Sanger sequencing, and SDS-PAGE to obtain desired genotypes. Two independent lines per genotype at the T4 generation were obtained: 7S^{KO}-1; 7S^{KO}-2; 7S^{KO}/cgs1^{KO}-1; 7S^{KO}/cgs1^{KO}-2; 7S^{KO}/cgs2^{KO}; 7S^{KO}/cgs1^{inf}-1; 7S^{KO}/cgs1^{inf}-2; 7s^{KO}/cgs2^{inf}-1; 7s^{KO}/cgs2^{inf}-2. The double in-frame GmCGS1/GmCCGS2 in 7S^{KO} background, 7s^{KO}/cgs1^{inf}/cgs2^{inf}, was obtained by crossing between line 7s^{KO}/cgs1^{inf}-1 and line 7s^{KO}/cgs2^{inf}-1 (FIG. 3A-B). This mutant was strongly resistant to DL-ethionine treatment at 300 μM compared to its parental lines and wild-type control. The same strategy was applied to generate the double KO gmcgs1/gmcgs2 in 7S^{KO} background; however, the line was not able to be obtained.

### Free Amino Acids Profiles and Protein Seed Profiles in Soybean

To investigate the effect of in-frame and frameshift mutations of CGS on methionine production, the transcript level of GmCGS1 and GmCGS2 and free methionine content in different tissues of all seven genotypes were examined. The expression level of GmCGS1 and GmCGS2 in leaves and green seeds were increased two- to three-fold in the in-frame mutants and undetectable in the frameshift mutants compared to the wild-type (FIG. 3F-I). The single GmCGS mutants showed slight increases or no difference in free methionine content compared to the wild-type control. However, three- to forty-six-fold increases in free methionine were obtained in leaves, roots, green seeds, and mature seed of the double in-frame 7s^{KO}/cgs1^{inf}/cgs2^{inf} mutant (FIG. 3J-L). These data indicated that GmCGS1 and GmCGS2 are functionally redundant for methionine biosynthesis in soybean. Besides increasing free Met, free asparagine, histidine, serine, and cysteine were also significantly increased in the mature seeds of the 7s^{KO}/cgs1^{inf}/cgs2^{inf} compared to the wild-type and other CRISPR lines.

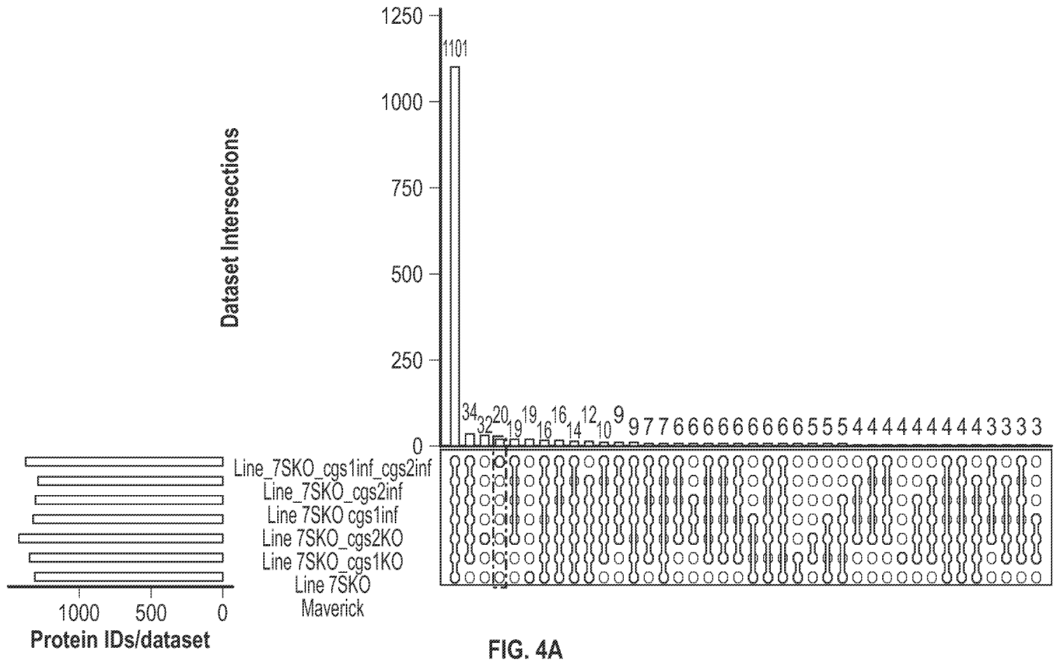

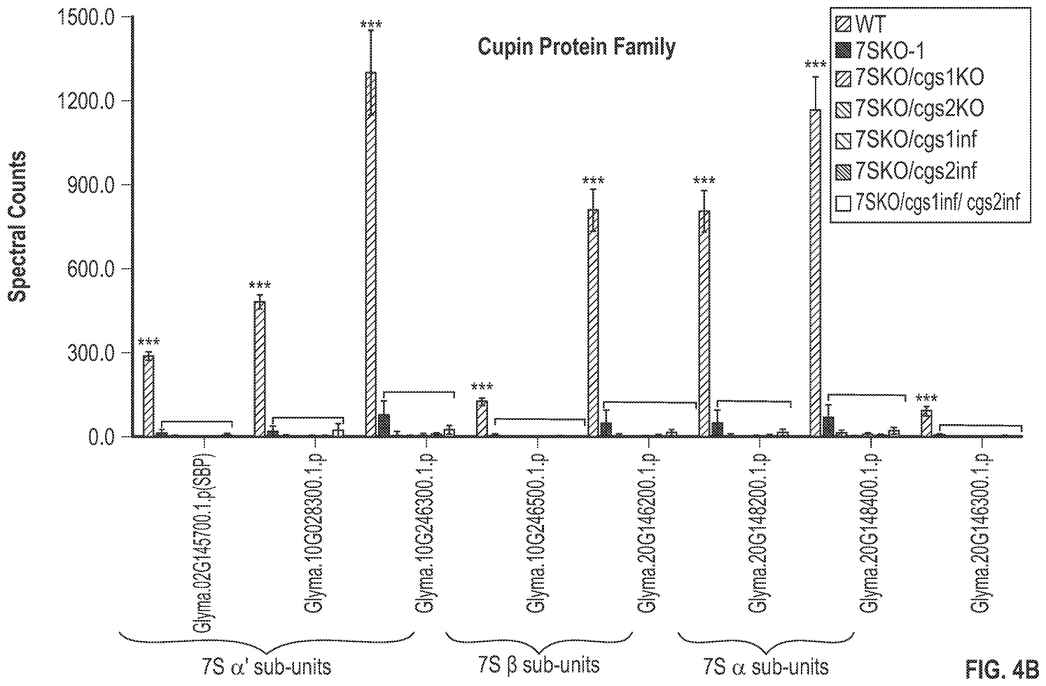

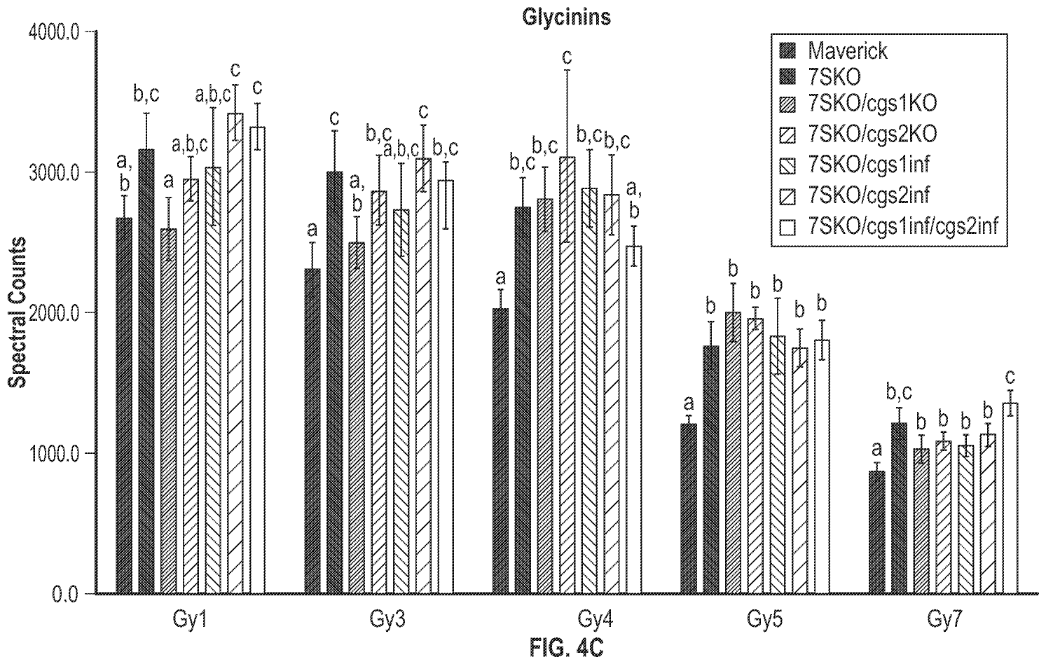

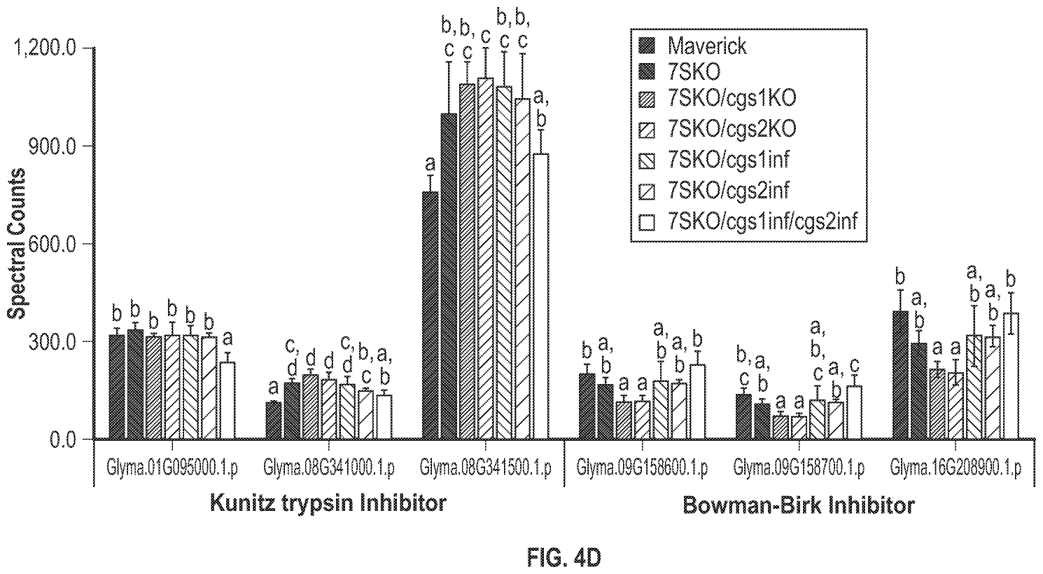

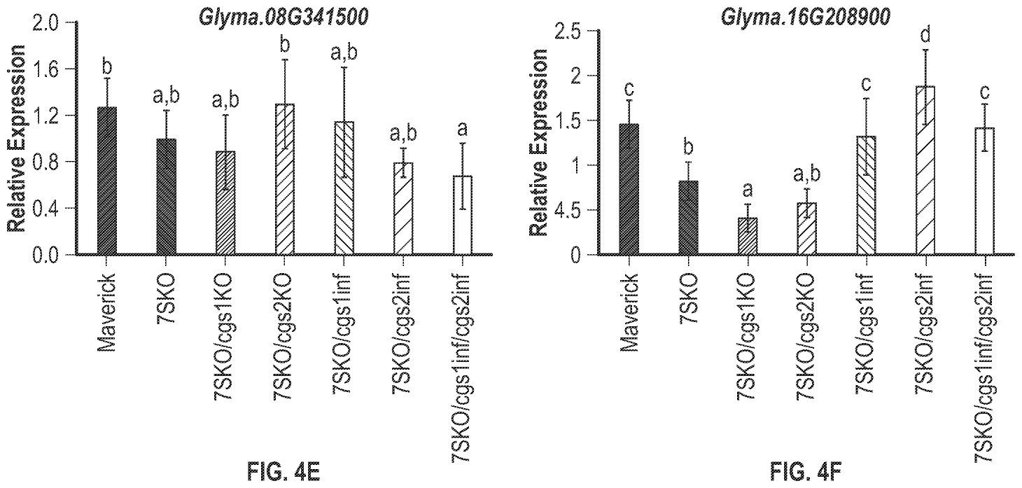

Next, a global quantitative proteomics approach was employed to investigate the effect of conglycinin and methionine on seed protein profiles in the mature soybean seeds. At a false discovery rate (FDR) of <1%, 1592 proteins passed these criteria: presence in at least three of five biological replicates with more than two spectrum counts per protein and identified in mature seeds of seven genotypes. The shared proteins among groups were then analyzed with UpSetR. About 70% of identified proteins were quantified in all seven genotypes while a few qualitative alterations were found in each genotype (FIG. 4A).

A total of 20 proteins were found only in the mature seed of the 7s^{KO}/cgs1^{inf}/cgs2^{inf} genotype, and they were involved in glutathione metabolisms pathway (FIG. 4A). Standard one-way ANOVA analysis controlled by a Benjamini-Hochberg FDR-corrected p<0.05 was performed and revealed 173 differential modulated proteins among different genotypes. Hierarchical clustering of these proteins based on abundance levels across different genotypes yielded a heatmap with clearly distinct protein clusters, especially compared between the wild-type and the 7s^{KO}/cgs1^{inf}/cgs2^{inf} CRISPR line. Gene ontology enrichment analysis (p<0.05) of the 173 significantly differential proteins revealed strong links to proteolysis, peptide biosynthesis, amide metabolic process and nutrient reservoir activity. The relative contribution of major protein groups to the total seed proteome showed untraceable 7S proteins in the seed proteome of all the CRISPR lines and greatly increased glycinins and other proteins (FIG. 4B-C). Intriguingly, the contribution of Kunitz trypsin inhibitors (KTI) and the Bowman-Birk protease inhibitors (BBI), other major soybean seed proteins, were strongly regulated by the excess of methionine content in seed proteome (FIG. 4D). KTI and BBI conventionally take ˜ 5% and 7% in seed proteome of wild-type, Maverick; while, KTI was accumulated to 7% and BBI was 2% reduction in the seed proteome of the 7S null mutant, 7S^{KO}. These effects were reversed in the seed proteome of the 7S^{KO}/cgs1^{inf}/cgs2^{inf} CRIPSR line of which KTI and BBI were 5% and 7%, no difference to wild-type seed proteome, respectively (FIG. 4E).

The mRNA levels of the major seed storage proteins among seven genotypes were investigated using qRT-PCR analysis on seed development samples when soybean seed storage proteins start to accumulate. The transcript levels of 7S, which were highly expressed in the wild-type, were undetectable in all CRISPR lines. Contrast to mRNA levels of glycinins which were not difference among genotypes, the expression levels of major KIT (Glyma.08g341500) and BBI (Glyma.16g208900) were significantly correlated with the present of methionine in the CRISPR lines (FIG. 4E-F). The data suggests, along with rebalancing, that the expression level of KIT and BBI are regulated by methionine content.

Increasing Methionine and Protein Content without Growth and Yield Defect in Soybean

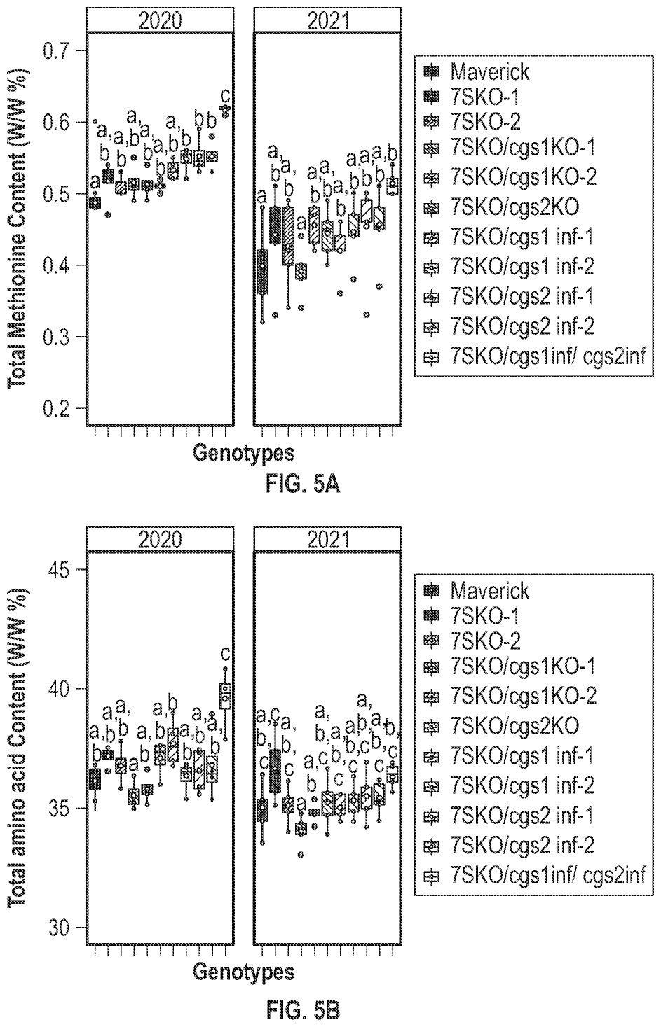

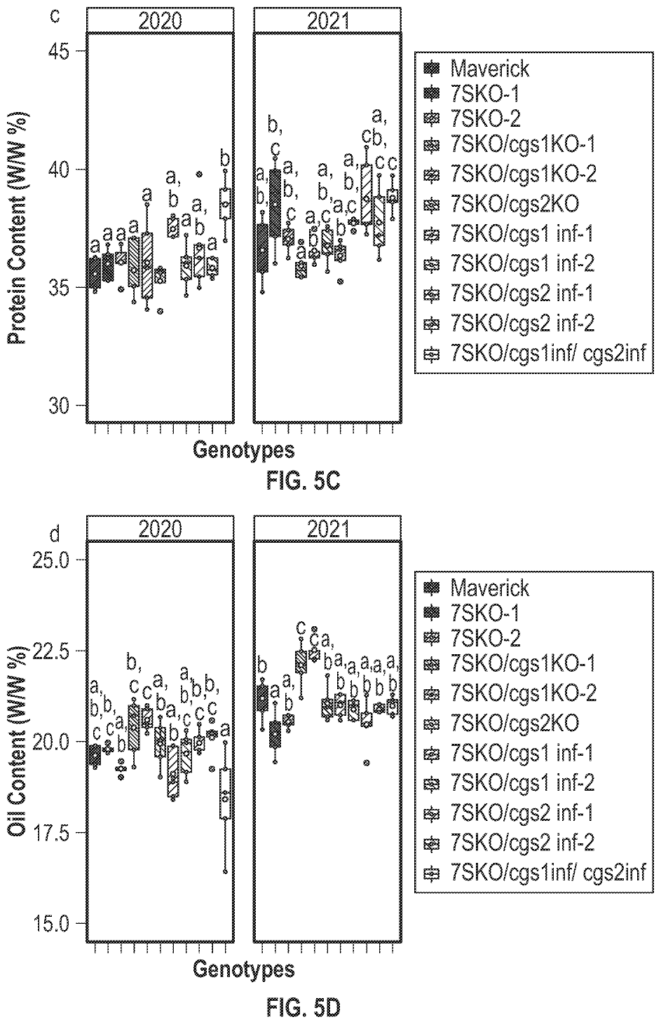

A standard protein hydrolysate analysis was performed to quantify and compare amino acid content in mature seeds of six genotypes with wild-type under field condition during 2-year period, 2020 and 2021. The null 7S alone or combined with single in-frame GmCGS mutant showed a little impact on the accumulation of total methionine content in seeds; however, they were not significant throughout two years. However, the total content of methionine of the null 7S and double GmCGS mutant, 7S^{KO}/cgs1^{inf}/cgs2^{inf} was ˜30% higher than wild-type in the 2 years (FIG. 5A). In addition, the 7S^{KO}/cgs1^{inf}/cgs2^{inf} produced higher amounts of threonine, alanine, glycine, tyrosine, and arginine with little impact on the other amino acids in mature seed, resulting in high accumulation of total amino acids in seed of this line (FIG. 5B). Spearman analysis indicated a positive correlation of the increasing methionine content with other amino acids in the mature seed of 7S^{KO}/cgs1^{inf}/cgs2^{inf} compared with the wild-type.

Protein and oil content were also analyzed in these samples. Seed protein content of the 7S^{KO}/cgs1^{inf}/cgs2^{inf} CRISPR line, in correlated with higher amino acid content, increased up to 3% as compared to wild-type control; whereas, no significant difference in seed protein contents was found in other CRISPR lines during 2 years analysis (FIG. 5C). Oil content was only slightly reduced or unchanged in the 7S^{KO}/cgs1^{inf}/cgs2^{inf} and 7S null mutants; however, the 7S-CGS1KO mutant lines produced ˜1.5% higher oil content in seed compared other lines (FIG. 5D). Fatty acid profiling revealed that oleic acid content was increased up to 8% higher than in of the 7S^{KO}/cgs1^{inf}/cgs2^{inf} compared to wild-type while the linoleic acid was reduced 7%. However, there was no difference in the transcript levels of GmFAD2-1, a major gene encoding for delta12 fatty acid desaturase 2 (FAD2) enzyme that catalyzes the conversion of oleic acid to linoleic acid.

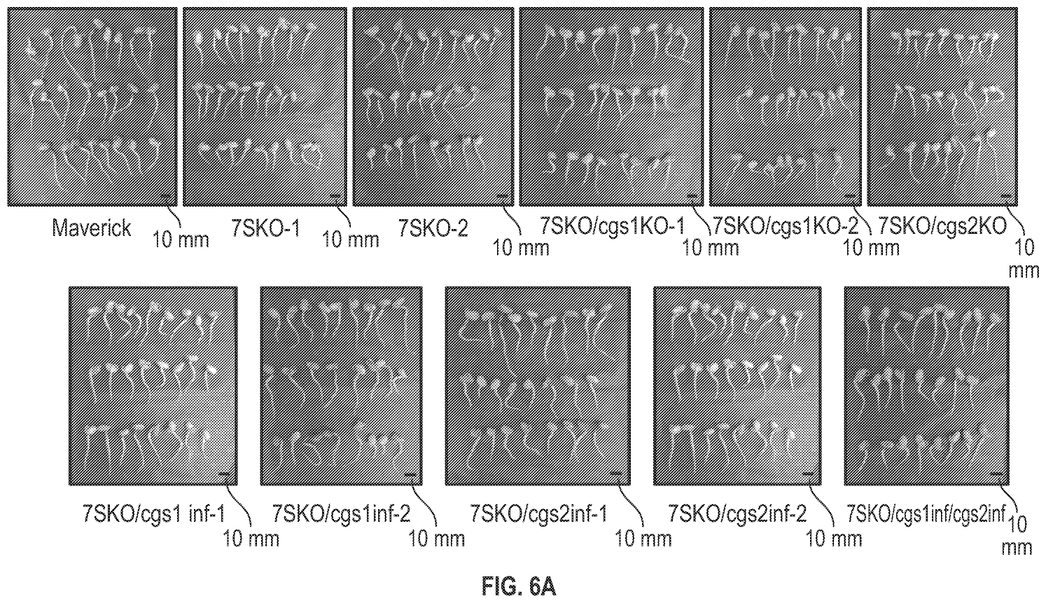

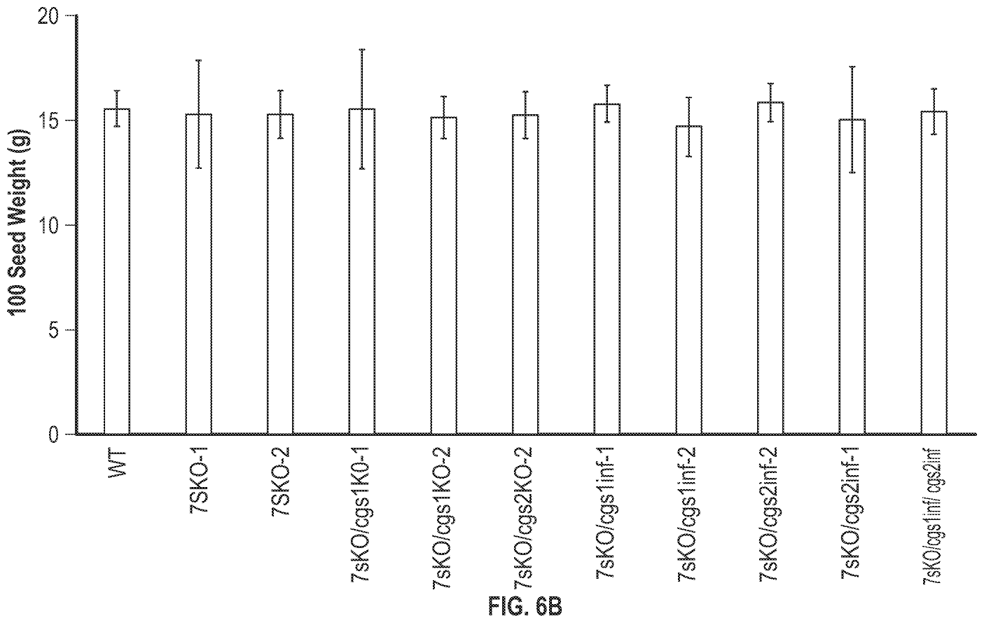

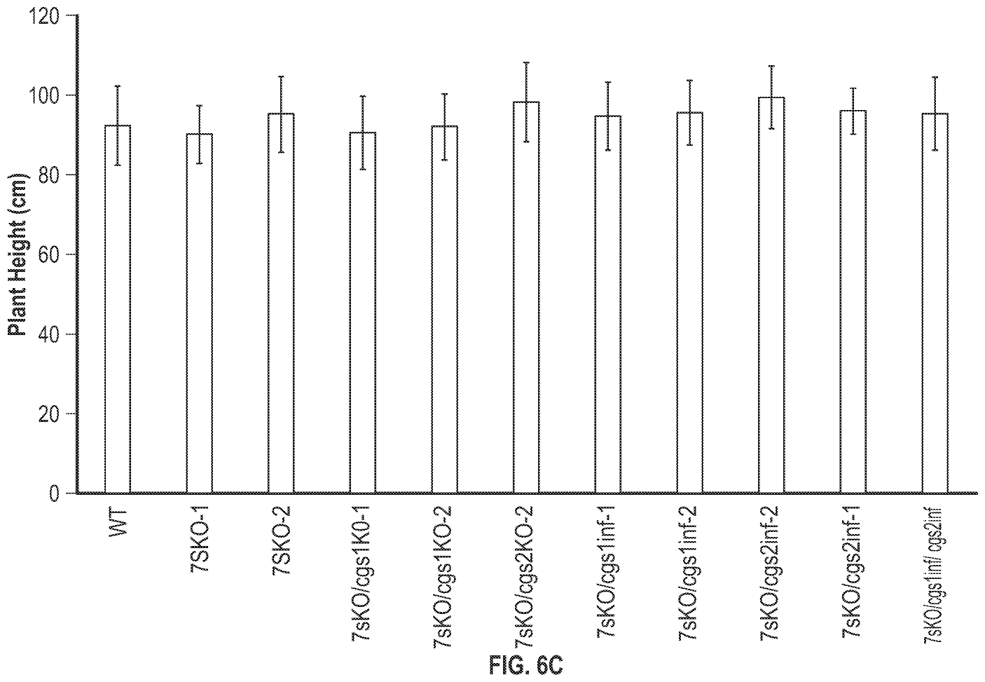

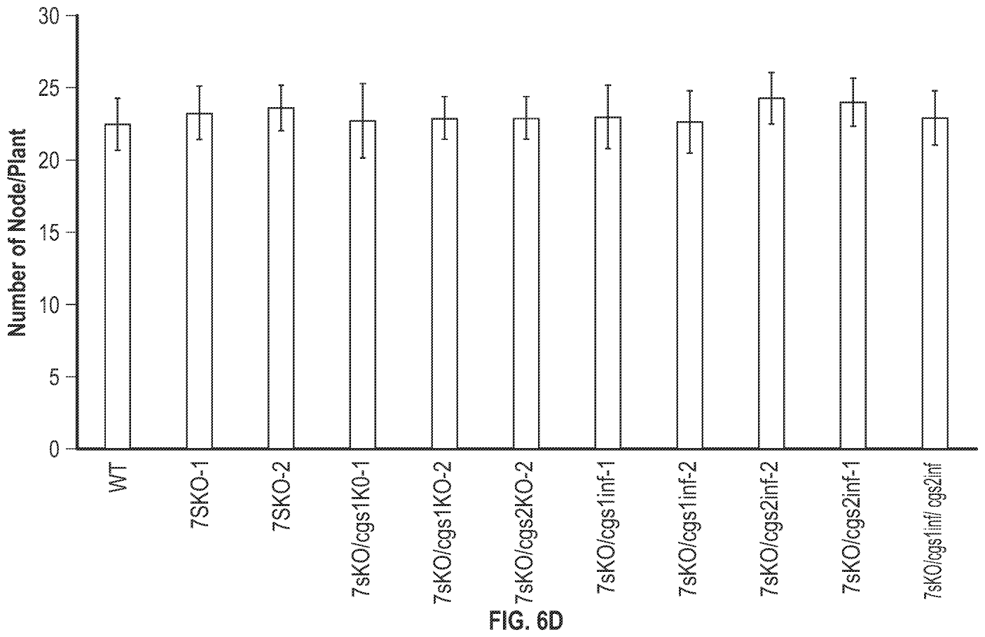

No differences in germination rates between the CRISPR lines and wild-type seeds were observed under growth chamber conditions (FIG. 6A). Lastly, the plant morphology, yield, and germination rate of the CRISPR lines were compared to wild-type. The CRISPR mutant lines displayed no growth penalties in terms of plant height, 100 seed weight, or grain yield under field conditions (FIG. 6B-D).

### DISCUSSION

Increasing methionine and protein content in seed with no growth defects is one of the major efforts in crop biotechnology and breeding in recent years. In this example, soybean lines with high methionine and protein in seed were generated in a short time period and with a single transformation event using the CRISPR/Cas9 editing system. In-frame mutations within and next to the MTO1 region of CGS (up to 27-base pair deletion in soybean) resulted in higher transcript abundance and free methionine in all tissues, which is a phenotypic mimic to the EMS mutants. The findings indicated that the in-frame GmCGS1 and GmCGS2 mutations interact synergistically in synthesizing methionine in soybean. The results also provide a new approach to generate high free methionine content crops through CRISPR/Cas9 approach other than expressing the feedback insensitive CGS mutant form into crops.

Increasing free Met alone is not sufficient to improve the total methionine in seeds.

Combining seed proteome rebalancing with high free Met through multiplex CRISPR/Cas9 editing boosted the total methionine and protein contents up to 30% and 3% in seeds, respectively. Null 7S mutants significantly changed the soybean seed proteome profile with increases in the accumulation of glycinins, lipoxygenase, KIT, and other seed proteins to compensate for the lack of 7S proteins. However, there is no impact on total methionine content. The results indicate soybean seeds have the capacity to compensate for a protein accumulation shortage by accumulating additional quantities of other seed proteins. Importantly, the data showed the availability of free methionine plays a significant role on regulating protein components in the rebalanced proteome in soybean seeds. While the GY were not strongly impacted by the free methionine at transcript and protein levels, methionine regulated the transcript levels of BBI, a major sulfur amino acid containing protein. The data suggests that regulation of free methionine at the transcription and post-transcriptional regulation of sulfur-rich amino acid containing proteins during proteome rebalancing are required to increasing methionine and protein content in soybean seeds. The approach developed from this example could be widely applied for nutritional quality crop improvement beyond methionine and protein content in seeds.

### REFERENCES

Alix, J. H. Molecular aspects of the in vivo and in vitro effects of ethionine, an analog of methionine. MICROBIOL. REV. 46, 281 (1982).
Chiba, Y. et al. Evidence for Autoregulation of Cystathionine γ-Synthase mRNA Stability in Arabidopsis. Science 286, 1371 (1999).
Chiba, Y. et al. S-adenosyl-methionine is an effector in the posttranscriptional autoregulation of the cystathionine γ-synthase gene in Arabidopsis. Proc Natl Acad Sci USA 100, 10225 (2003).
Conway, J. R., Lex, A. & Gehlenborg, N. UpSetR: an R package for the visualization of intersecting sets and their properties. Bioinformatics 33, 2938-2940 (2017).
Czechowski, T., Stitt, M., Altmann, T., Udvardi, M. K. & Scheible, W.-R. Genome-Wide Identification and Testing of Superior Reference Genes for Transcript Normalization in Arabidopsis. Plant Physiol. 139, 5 (2005).
Dahal, D., Mooney, B. P. & Newton, K. J. Specific changes in total and mitochondrial proteomes are associated with higher levels of heterosis in maize hybrids. The Plant Journal 72, 70-83 (2012).
Dancs, G., Kondrák, M. & Bánfalvi, Z. The effects of enhanced methionine synthesis on amino acid and anthocyanin content of potato tubers. BMC Plant Biology 8, 65 (2008).
Do, P. T. et al. Demonstration of highly efficient dual gRNA CRISPR/Cas9 editing of the homeologous GmFAD2-1A and GmFAD2-1B genes to yield a high oleic, low linoleic and α-linolenic acid phenotype in soybean. BMC Plant Biology 19, 311 (2019).
Galili, G., Amir, R. & Fernie, A. R. The Regulation of Essential Amino Acid Synthesis and Accumulation in Plants. Annu. Rev. Plant Biol. 67, 153-178 (2016).
Hacham, Y., Avraham, T. & Amir, R. The N-Terminal Region of Arabidopsis Cystathionine γ-Synthase Plays an Important Regulatory Role in Methionine Metabolism. Plant Physiol. 128, 454 (2002).
Hacham, Y., Schuster, G. & Amir, R. An in vivo internal deletion in the N-terminus region of Arabidopsis cystathionine γ-synthase results in CGS expression that is insensitive to methionine. The Plant Journal 45, 955-967 (2006).
Hagiwara-Komoda, Y., Sugiyama, T., Yamashita, Y., Onouchi, H. & Naito, S. The N-Terminal Cleavable Pre-Sequence Encoded in the First Exon of Cystathionine γ-Synthase Contains Two Different Functional Domains for Chloroplast Targeting and Regulation of Gene Expression. Plant and Cell Physiology 55, 1779-1792 (2014).
Hanafy, M. S. et al. Differential response of methionine metabolism in two grain legumes, soybean and azuki bean, expressing a mutated form of Arabidopsis cystathionine γ-synthase. Journal of Plant Physiology 170, 338-345 (2013).
Harada, J. J., Barker, S. J. & Goldberg, R. B. Soybean beta-conglycinin genes are clustered in several DNA regions and are regulated by transcriptional and posttranscriptional processes. Plant Cell 1, 415 (1989).
Hesse, H. & Hoefgen, R. Molecular aspects of methionine biosynthesis. Trends in Plant Science 8, 259-262 (2003).
Hu, R., Fan, C., Li, H., Zhang, Q. & Fu, Y.-F. Evaluation of putative reference genes for gene expression normalization in soybean by quantitative real-time RT-PCR. BMC Molecular Biology 10, 93 (2009).
Inaba, K. et al. Isolation of an Arabidopsis thaliana Mutant, mto1, That Overaccumulates Soluble Methionine (Temporal and Spatial Patterns of Soluble Methionine Accumulation). Plant Physiol. 104, 881 (1994).
Kim, W.-S., Jez, J. M. & Krishnan, H. B. Effects of proteome rebalancing and sulfur nutrition on the accumulation of methionine rich 8-zein in transgenic soybeans. Frontiers in Plant Science 5, 633 (2014).
Krishnan, H. B. & Jez, J. M. Review: The promise and limits for enhancing sulfur-containing amino acid content of soybean seed. Plant Science 272, 14-21 (2018).
Kwanyuen, P., Pantalone, V. R., Burton, J. W. & Wilson, R. F. A new approach to genetic alteration of soybean protein composition and quality. Journal of the American Oil Chemists' Society 74, 983-987 (1997).
Libault, M. et al. Identification of Four Soybean Reference Genes for Gene Expression Normalization. The Plant Genome 1, 44-54 (2008).
Lin, J.-Y. et al. Similarity between soybean and <em>Arabidopsis</em> seed methylomes and loss of non-CG methylation does not affect seed development. Proc Natl Acad Sci USA 114, E9730 (2017).
Loizeau, K. et al. Regulation of One-Carbon Metabolism in Arabidopsis: The N-Terminal Regulatory Domain of Cystathionine γ-Synthase Is Cleaved in Response to Folate Starvation. Plant Physiol. 145, 491 (2007).
Nguyen, C. X., Paddock, K. J., Zhang, Z. & Stacey, M. G. GmKIX8-1 regulates organ size in soybean and is the causative gene for the major seed weight QTL qSw17-1. New Phytologist 229, 920-934 (2021).
Nielsen, N. C. et al. Characterization of the glycinin gene family in soybean. Plant Cell 1, 313 (1989).
Ominato, K. et al. Identification of a Short Highly Conserved Amino Acid Sequence as the Functional Region Required for Posttranscriptional Autoregulation of the Cystathionine γ-Synthase Gene in Arabidopsis. Journal of Biological Chemistry 277, 36380-36386 (2002).
Onouchi, H. et al. Nascent peptide-mediated translation elongation arrest coupled with mRNA degradation in the CGS1 gene of Arabidopsis. Genes & Development 19, 1799-1810 (2005).
Onoue, N. et al. S-Adenosyl-1-methionine Induces Compaction of Nascent Peptide Chain inside the Ribosomal Exit Tunnel upon Translation Arrest in the Arabidopsis CGS1 Gene. Journal of Biological Chemistry 286, 14903-14912 (2011).
Raudvere, U. et al. g: Profiler: a web server for functional enrichment analysis and conversions of gene lists (2019 update). Nucleic Acids Research 47, W191-W198 (2019).
Ravanel, S., GAKIERE, B., JOB, D. & DOUCE, R. Cystathionine γ-synthase from Arabidopsis thaliana: purification and biochemical characterization of the recombinant enzyme overexpressed in Escherichia coli. Biochemical Journal 331, 639-648 (1998).
Schmidt, M. A. et al. Silencing of Soybean Seed Storage Proteins Results in a Rebalanced Protein Composition Preserving Seed Protein Content without Major Collateral Changes in the Metabolome and Transcriptome. Plant Physiol. 156, 330 (2011).
Song, S. et al. Soybean seeds expressing feedback-insensitive cystathionine γ-synthase exhibit a higher content of methionine. Journal of Experimental Botany 64, 1917-1926 (2013).
Stemmer, M., Thumberger, T., del Sol Keyer, M., Wittbrodt, J. & Mateo, J. L. CCTop: An Intuitive, Flexible and Reliable CRISPR/Cas9 Target Prediction Tool. PLOS ONE 10, e0124633 (2015).
Whitcomb, S. J., Nguyen, H. C., Brückner, F., Hesse, H. & Hoefgen, R. CYSTATHIONINE GAMMA-SYNTHASE activity in rice is developmentally regulated and strongly correlated with sulfate. Plant Science 270, 234-244 (2018).
Yamada, T. et al. Knockdown of the 7S globulin subunits shifts distribution of nitrogen sources to the residual protein fraction in transgenic soybean seeds. Plant Cell Reports 33, 1963-1976 (2014).
Yamashita, Y. et al. Ribosomes in a Stacked Array: ELUCIDATION OF THE STEP IN TRANSLATION ELONGATION AT WHICH THEY ARE STALLED DURING S-ADENOSYL-1-METHIONINE-INDUCED TRANSLATION ARREST OF CGS1 mRNA. Journal of Biological Chemistry 289, 12693-12704 (2014).
Yobi, A. & Angelovici, R. A High-Throughput Absolute-Level Quantification of Protein-Bound Amino Acids in Seeds. Current Protocols in Plant Biology 3, e20084 (2018).
Yu, Y. et al. Constitutive expression of feedback-insensitive cystathionine γ-synthase increases methionine levels in soybean leaves and seeds. Journal of Integrative Agriculture 17, 54-62 (2018).

What is claimed is:

1. A plant, plant part, or seed comprising one or more modifications that increase seed protein content, the modification selected from a deletion, insertion, or substitution of a nucleotide in (i) a genomic sequence encoding a cystathionine gamma-synthase (CGS), the modification resulting in suppression of the feedback regulation of the same while retaining enzymatic activity, and/or (ii) a genomic sequence encoding a low methionine content seed storage protein, the modification resulting in suppression of the activity of the same.

2. The plant, plant part, or seed of claim 1, wherein the modification comprises a deletion, insertion, or substitution of a nucleotide at or near the MTO1 region of the genomic sequence encoding the CGS.

3. The plant, plant part, or seed of claim 1, wherein the modification is an in-frame modification of the genomic sequence encoding the CGS.

4. The plant, plant part, or seed of claim 1, wherein the deletion or insertion results in a frameshift mutation of the genomic sequence encoding the low methionine content seed storage protein.

5. The plant, plant part, or seed of claim 1, wherein the plant, plant part, or seed comprises a modification selected from a deletion, insertion, or substitution of a nucleotide in a genomic sequence encoding a 7S storage protein.

6. The plant, plant part, or seed of claim 5, wherein the plant, plant part, or seed comprises a modification selected from a deletion, insertion, or substitution of a nucleotide in a genomic sequence encoding an alpha′, alpha, and beta subunit of the 7S storage protein.

7. The plant, plant part, or seed of claim 1, further comprising a heterologous nucleic acid sequence selected from a reporter gene, a selection marker, a disease resistance gene, a herbicide resistance gene, an insect resistance gene, a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance, and a gene involved in salt resistance in plants.

8. The plant, plant part, or seed of claim 1, wherein protein content of the seed is increased relative to a control seed not comprising the modification.

9. The plant, plant part, or seed of claim 1, wherein methionine content of the seed is increased relative to a control seed not comprising the modification.

10. The plant, plant part, or seed of claim 1, wherein proteome rebalancing results in an increase in high methionine seed storage proteins in the seed.

11. The plant, plant part, or seed of claim 1, wherein 11S glycinin polypeptide content of the seed is increased relative to a control seed not comprising the modification as a result of proteome rebalancing.

12. The plant, plant part, or seed of claim 1, wherein the modification is homozygous.

13. The plant, plant part, or seed of claim 1, wherein the genomic sequence encoding the CGS comprises a nucleotide sequence having at least 80%, at least 90%, at least 95%, at least 98%, at least 99%, or 100% sequence identity to SEQ ID NO: 1 or 4.

14. The plant, plant part, or seed of claim 1, wherein the genomic sequence encoding the low methionine content seed storage protein comprises a nucleotide sequence having at least 80%, at least 90%, at least 95%, at least 98%, at least 99%, or 100% sequence identity to SEQ ID NO: 7, 10, 13, 16, 19, 22, 25, or 28.

15. A method of making a modified plant with high seed protein content compared to the seed protein content of a nonmodified plant, the method comprising: (a) crossing a first plant comprising a deletion, insertion, or substitution of a nucleotide in (i) a genomic sequence encoding cystathionine gamma-synthase (CGS), the modification resulting in suppression of the feedback regulation of the same while retaining enzymatic activity, and/or (ii) a genomic sequence encoding a low methionine content seed storage protein, the modification resulting in suppression of the activity of the same, with a second plant; and (b) selecting a progeny plant, or plant part thereof, with high protein content for further breeding.

16. The method of claim 15, wherein the modified plant is an F1 progeny plant.

17. A method of producing high protein seed meal comprising: harvesting the seed of claim 1 from a plant comprising the modification and processing the seed to form meal.

18. A modified CGS polynucleotide comprising an in-frame insertion or deletion in the MTO1 region so that feedback inhibition of expression is suppressed.

19. The modified CGS polynucleotide of claim 18, wherein the polynucleotide comprises the sequence of SEQ ID NO: 44, 45, 46, 48, 49, 50, or 51.

20. The modified CGS polynucleotide of claim 18, wherein the polynucleotide comprises a sequence having at least 80%, at least 90%, at least 95%, at least 98%, at least 99%, or 100% sequence identity to SEQ ID NO: 52, 53, 54, 56, 58, 60, or 61.

21. A modified 7S polynucleotide comprising an insertion, deletion, or substitution that suppresses activity of the polynucleotide.

22. The modified 7S polynucleotide of claim 21, wherein the polynucleotide comprises a sequence having at least 80%, at least 90%, at least 95%, at least 98%, at least 99%, or 100% sequence identity to one or more of SEQ ID NOs: 62-108.

23. A plant cell comprising the polynucleotide of claim 18 or claim 21 or both.

24. The plant cell of claim 23, wherein the plant cell is a soybean plant cell.

25. A plant comprising the plant cell of claim 23.

26. A seed of the plant of claim 25, wherein the seed has increased protein content compared to a seed comprising a comparable polynucleotide without the modification.

27. A seed of the plant of claim 25, wherein the seed has increased methionine content compared to a seed comprising a comparable polynucleotide without the modification.

28. A modified polypeptide encoded by the polynucleotide of claim 18 or claim 21.

29. A method of producing a plant cell having increased protein content, the method comprising: introducing a double-strand-break-inducing (DSB-inducing) agent to a plant cell, wherein the DSB-inducing agent creates a double-strand break in an endogenous CGS gene and/or a low methionine content seed storage protein gene in the genome of the plant, wherein the repair of the DSB results in the production of a modified polynucleotide, and wherein the production of the modified polynucleotide produces increased protein content in the seed.

30. The method of claim 29, wherein the modified polynucleotide includes a deletion, insertion, or substitution in the MTO1 region of the CGS gene.

31. The method of claim 30, wherein the deletion or insertion is an in-frame deletion or insertion.

32. The method of claim 29, wherein the modified polynucleotide is a deletion, insertion, or substitution of a 7S beta-conglycinin.

33. The method of claim 29, wherein the endogenous CGS gene comprises a nucleotide sequence having at least 80%, at least 90%, at least 95%, at least 98%, at least 99%, or 100% sequence identity to SEQ ID NO: 1 or 4.

34. The method of claim 29, wherein the endogenous low methionine content seed storage protein gene comprises a nucleotide sequence having at least 80%, at least 90%, at least 95%, at least 98%, at least 99%, or 100% sequence identity to SEQ ID NO: 7, 10, 13, 16, 19, 22, 25, or 28.

35. A method of editing the genome of a plant cell to produce a plant cell having increased protein, the method comprising: introducing a guide RNA, a polynucleotide modification template, and a Cas endonuclease to a plant cell; and regenerating a plant from the plant cell wherein the genome of the regenerated plant comprises the modified polynucleotide of claim 18 or claim 21 or both.

36. The method of claim 35, wherein the seed of the plant has increased protein content compared with a comparable seed not comprising the modified polynucleotide.

37. The method of claim 35, wherein the seed of the plant has increased methionine content compared with a comparable seed not comprising the modified polynucleotide.

38. The method of claim 35, wherein the genome is modified by a site-directed endonuclease.

39. The method of claim 35, wherein the plant cell is a soybean cell.

40. A method of producing a plant cell having increased seed protein content, the method comprising: transforming a plant cell with the modified polynucleotide of claim 18 or claim 21 or both, wherein a plant regenerated from the transformed plant cell has increased seed protein content compared with a comparable plant cell not comprising the modified polynucleotide.

41. A polynucleotide construct comprising a sequence encoding a guide RNA which targets an endogenous CGS gene and/or a low methionine content storage polypeptide gene when expressed in a plant cell and thereby generates a modified endogenous CGS gene and/or a low methionine content storage polypeptide gene.

42. The polynucleotide construct of claim 41, wherein the guide RNA comprises the spacer sequence of SEQ ID NO: 109, 110, 111, 112, 113, or 114.

43. A plant cell comprising the polynucleotide construct of claim 41.

44. The plant cell of claim 43, wherein the plant cell is a soybean cell.

45. A method of producing a soybean seed comprising: a) sexually crossing a first soybean line comprising the polynucleotide of claim 18 or claim 21 or both with a second soybean line not comprising the polynucleotide; and b) harvesting the seed produced thereby.

46. The method of claim 45 further comprising the step of backcrossing a second generation progeny plant that comprises the polynucleotide to the parent plant that lacks the polynucleotide, thereby producing a backcross progeny plant that produces seed with increased protein content.

47. A method of screening for the presence or absence of the polynucleotide of claim 18 or claim 21 or both in a plurality of genomic soybean DNA samples, the method comprising the steps of (a) contacting a plurality of genomic soybean DNA samples, at least some of which comprise the polynucleotides, with a first DNA primer molecule and a second DNA primer molecule; (b) providing a plurality nucleic acid amplification reaction conditions; (c) performing the nucleic acid amplification reactions, thereby producing a DNA amplicon molecule indicating the presence of the polynucleotide or a wild-type CGS and/or a low methionine content storage polypeptide gene nucleotide sequence; and (d) detecting the DNA amplicon molecules, wherein the presence, absence or size of the DNA amplicon molecule indicates the presence or absence of the polynucleotide in the at least one of the plurality of genomic soybean DNA samples.

48. The method of claim 47, wherein the first DNA primer molecule or the second DNA primer molecule comprises the sequence of one or more of SEQ ID NOs: 115-134.

49. An animal feed comprising the plant, plant part, or seed of claim 1.
